(19) United States
(12) Patent Application Publication
(10) Pub. No.: US 2023/0071889 A1
(43) Pub. Date: Mar. 9, 2023

(54) BIFUNCTIONAL ANTI-PD-1/IL-7 MOLECULE

(71) Applicant: OSE IMMUNOTHERAPEUTICS, NANTES (FR)

(72) Inventors: NICOLAS POIRIER, TREILLIERES (FR); CAROLINE MARY, SAINTE PAZANNE (FR); AURORE MORELLO, SAINT SEBASTIEN SUR LOIRE (FR); JUSTINE DURAND, NANTES (FR)

(21) Appl. No.: 17/414,970

(22) PCT Filed: Dec. 17, 2019
(86) PCT No.: PCT/EP2019/085791

§ 371 Date: Jun. 17, 2021

(30) Foreign Application Priority Data
Dec. 21, 2018 (EP) 18306808.9

(51) Int. Cl. C07K 16/28; C07K 14/54; A61P 35/00; A61K 45/06; A61K 38/20; A61K 39/395

(52) U.S. Cl. CPC C07K 16/2818; C07K 14/5418; A61P 35/00; A61K 45/06; A61K 38/2046; A61K 39/3955; A61K 2039/505

(57) ABSTRACT

The present invention relates to a bifunctional molecule comprising an anti-PD-1 antibody and IL-7 and its uses.

### CROSS-REFERENCE TO RELATED APPLICATION

This application is the U.S. national stage application of International Patent Application No. PCT/EP2019/085791, filed Dec. 17, 2019.

### STATEMENT REGARDING SEQUENCE LISTING

The Sequence Listing for this application is labeled “Seq-List.txt” which was created on Jun. 8, 2021 and is 91 KB. The entire content of the sequence listing is incorporated herein by reference in its entirety.

### FIELD OF THE INVENTION

The invention pertains to the field of immunotherapy. The present invention provides a bifunctional molecule that comprises an anti-PD1 antibody or antibody fragment thereof.

### BACKGROUND OF THE INVENTION

The approach of targeting T cell inhibition checkpoints for dis-inhibition with therapeutic antibodies is an area of intense investigation (for a review, see Pardoll, Nat Rev Cancer. 2012; 12:253-264). Targeting immune checkpoints of the adaptive immunity has shown great therapeutic efficacy to fight numerous cancers, but in a limited proportion of patients. Combining immune checkpoint therapies with other immunotherapeutic strategies has demonstrated great efficiency in preclinical models but remains a challenge in clinic.

Immune cells activation is governed by the integration of balance co-stimulatory and co-inhibitory signals. T cell receptor (TCR)-mediated T cell activation is modulated by both co-stimulatory and co-inhibitory signals. The antigen-independent second signal modifies first signal, provided by interaction of antigenic peptide-MHC complex with the TCR, which confers specificity to the response. T cell co-stimulatory and co-inhibitory pathways have a broad immunoregulatory functions, controlling effector, memory and regulatory T cells, as well as naive T cells. Therapeutic modulation of those pathways is translating to effective new strategies for treating cancer (For review, see Schildberg et al., 44(5), Immunity, 2016). Ongoing studies on regulation of the immune responses have led to the identification of multiple immunologic pathways that may be targeted for the development of cancer therapies. Those molecules are referred herein as immune checkpoint co-activators or co-inhibitors (see review Sharma et al., Cell, 161(2), 2015 and Pardoll, Nature Reviews Cancer, 12(4), 2012).

Programmed cell death protein 1 (PD-1, also known as CD279) is a cell surface protein molecule that belongs to the immunoglobulin superfamily. It is expressed on T and B lymphocytes and macrophages, and plays a role in cell fate and differentiation. Two ligands for PD-1 have been identified, PD-L1 and PD-L2, that have been shown to downregulate T cell activation upon binding to PD-1 (Freeman et al. (2000) J Exp Med 192: 1027-34; Latchman et al. (2001) Nat Immunol 2:261-8; Carter et al. (2002) Eur J Immunol 32:634-43). The interaction between PD-1 and its ligand results in a decrease in tumor infiltrating lymphocytes, a decrease in T-cell receptor mediated proliferation, and immune evasion by the cancerous cells. Particularly, PD1 ligation reduces signals downstream of TCR stimulation on T cells, inhibiting T cell response and resulting in decreased activation and cytokine production.

PD-1/PD-L1 therapy has been approved by the FDA for the treatment as first- and second-line therapy for large broad hematological and solid cancers but objective response based on the reduction in tumor size greater 30% as defined by RECIST criteria is highly variable between cancer subtypes.

High response rate was observed in refractory Hodgkin's lymphoma (65-85%) (Borcherding N et al J Mol Biol. 2018 Jul. 6; 430(14):2014-2029) or in tumor with high microsatellite instability colon carcinoma (MSI-H, 25%-80%) or Merkel cell carcinoma (56%).

A mid objective response rate is observed in melanoma (24 to 44%) and non-small cell lung cancer patients (12.8 to 43,7%), where anti PD-1 therapy is used as first line treatment. Although only a portion of patient benefits from the therapy, PD-1/PD-L1 therapy improved overall survival compared to old standard care chemotherapy.

In some solid tumors, low or no clinical response were observed, notably in pancreatic cancer, non MSI colorectal cancer, gastric cancer and some breast cancer (Borcherding N et al J Mol Biol. 2018 Jul. 6; 430(14):2014-2029).

Multiple mechanisms have been described and may explain this differential efficacy and resistance to PD-1/PD-L1 checkpoint therapy, in particular several of them concern T-cell biology such as (1) impaired formation of memory T-cells (2) impaired T cell infiltration (3) insufficient generation of tumor specific T cells (4) inadequate function of T cells and (5) immunosuppressive microenvironment induced by regulatory T cells. Combining treatment by targeting IL-7 signaling may be a good strategy to overcome anti-PD-1 resistant patient by stimulating T cell infiltration, sustaining T cell effector capacity and promoting a long-lasting memory T cell response without stimulating regulatory T cells expansion and survival.

Interleukin-7 is an immunostimulatory cytokine member of the IL-2 superfamily plays an important role in an adaptive immune system and promote immune responses mediated by B cells and T cells. This cytokine activates immune functions through the survival and differentiation of T cells and B cells, survival of lymphoid cells, stimulation of activity of natural killer (NK) cell. IL-7 also regulates the development of lymph nodes through lymphoid tissue inducer (LTi) cells and promotes the survival and division of naive T cells or memory T cells. Furthermore, IL-7 enhances immune response in human by promoting the secretion of IL-2 and Interferon-γ. The receptor of IL-7 is heterodimeric and consists of the IL-7Rα (CD127) and the common γ chain (CD132). The y chain is expressed on all hematopoietic cell types whereas IL-7Rα is mainly expressed by lymphocytes that includes B and T lymphoid precursors, naïve T cells and memory T cell. A low expression of IL-7Rα is observed on regulatory T cells compared to effector/naive T cells that express a higher level, thereby CD127 is used as surface marker to discriminate these 2 populations. IL-7Rα is also expressed on Innate lymphoid cells as NK and gut-associated lymphoid tissue (GALT)-derived T cells. IL-7Rα (CD127) chain is shared with TSLP (Tumor stromal lymphopoietin) and CD132 is shared with IL-2, IL-4, IL-9, IL-15 and interleukin-21. Two main signaling pathways are induced through CD127/CD132 (1) Janus kinase/STAT pathway (i.e. Jak-Stat-3 and 5) and (2) the phosphatidyl-inositol-3kinase pathway (i.e. PI3K-Akt). IL-7 administration is well tolerated in patient and leads to CD8 and CD4 cell expansion and a relative decrease of CD4+ T regulatory cells. Recombinant naked IL-7 or IL-7 fused to N terminal domain of the Fc of antibodies have been tested in clinic, with the rationale to increase IL-7 half-life via fusion of the Fc domain and enhance long lasting efficiency of the treatment. Targeting IL-7 signaling should hold greater promise compare to IL-2 signaling as IL-2 acts on both Treg and T effector cells whereas IL-7 selectively activates T effector cells.

To increase the efficacy of anti-PD1 immunotherapy and overcome potential anti PD-1 resistance in patient, the development of a combination treatment targeting IL-7 signaling may be a good strategy to stimulate T cell infiltration, sustaining T cell effector capacity and promoting a long-lasting memory T cell response without stimulating regulatory T cells expansion and survival. Indeed, anti PD-1 therapy increases expression of CD127 on exhausted T cells thereby increasing their capacity to respond to IL-7 and improved coproduction of interferon-γ (IFN-γ) and tumor necrosis factor-α (TNF-α) (Pauken et al., Science. 2016 Dec. 2; 354(6316):1160-1165, Shi et al., Nat Commun. 2016 Aug. 8; 7:12335). However, the validation and development of combined immunotherapies are strongly limited by the cost of biotherapies and the limited access to such immunotherapies. There remains therefore a significant need in the art for new and improved agents for safe immunotherapy, notably against cancer, targeting T cells with an effective positive impact on adaptive immune response, in particular T cell immune responses. The present inventors have made a significant step forward with the invention disclosed herein.

### SUMMARY OF THE INVENTION

The inventors provide a bifunctional molecule comprising an anti-hPD-1 antibody and a human IL-7 promising for numerous therapeutic applications, in particular for the treatment of cancer. The present invention is based on the development of an antibody specifically targeting human PD-1 which shows high binding affinity to PD-1 and strong competition with its ligands PD-L1 and PD-L2. Surprisingly, the fusion of the N-terminal end of the IL-7 to the C-terminal end of the Fc region of the anti-hPD-1 antibody allows the conservation of its high affinity for CD127 (the IL7 Receptor) to similar extend to endogenous IL-7, suggesting a potent IL-7R activation. The fusion of the Fc domain to IL-7 also increases the product half-life. Furthermore, the bifunctional anti-PD1/IL-7 molecule disclosed herein allows accumulation of IL-7 in PD-1+ T cells infiltrates and re-localization of IL-7 on PD-1+ T cells. Particularly, the anti-PD-1/IL-7 bifunctional molecule induces the proliferation and activation of naïve, partially exhausted and fully exhausted T-cell subsets reflected by cytokine (e.g. IFNγ) secretion and integrin (e.g. Alpha4 and Beta7 and LFA-1) expression. Such anti-hPD-1/IL-7 bifunctional molecule has the capacity to overcome associated resistance mechanism and improve efficacy of anti PD-1 immunotherapies.

In a first aspect, the invention concerns a bifunctional molecule that comprises:

(a) an anti-human PD-1 antibody or an antigen-binding fragment thereof, which comprises:
(i) a heavy chain variable domain (VH) comprising a HCDR1, a HCDR2 and a HCDR3, and
(ii) a light chain variable domain (VL) comprising a LCDR1, a LCDR2 and a LCDR3, and

(b) a human interleukin 7 (IL-7) or a fragment thereof,

wherein the antibody or the fragment thereof is covalently linked to the human IL-7 or a fragment thereof as a fusion protein, preferably by a peptide linker.

Particularly, the N-terminal end of the human IL-7 or the fragment thereof is connected to the C-terminal end of the heavy chain or of the light chain of the anti-human PD-1 antibody or the antigen-binding fragment thereof or both.

In one aspect, the antibody or the antigen-binding fragment thereof is a chimeric, a humanized or a human antibody.

In a particular aspect, the invention relates to a bifunctional molecule comprises an anti-human PD-1 antibody or antigen-binding fragment thereof, that comprises or consists of:
(i) a heavy chain variable domain (VH) comprising HCDR1, HCDR2 and HCDR3, and
(ii) a light chain variable domain (VL) comprising LCDR1, LCDR2 and LCDR3,
   wherein:
the heavy chain CDR1 (HCDR1) comprises or consists of an amino acid sequence of SEQ ID NO: 1;
the heavy chain CDR2 (HCDR2) comprises or consists of an amino acid sequence of SEQ ID NO: 2;
the heavy chain CDR3 (HCDR3) comprises or consists of an amino acid sequence of SEQ ID NO: 3;
wherein X1 is D or E and X2 is selected from the group consisting of T, H, A, Y, N, E and S, preferably in the group consisting of H, A, Y, N, and E;
the light chain CDR1 (LCDR1) comprises or consists of an amino acid sequence of SEQ ID NO: 12 wherein X is G or T;
the light chain CDR2 (LCDR2) comprises or consists of an amino acid sequence of SEQ ID NO: 15,
the light chain CDR3 (LCDR3) comprises or consists of an amino acid sequence of SEQ ID NO:16.

Particularly, the anti-human PD-1 antibody or antigen-binding fragment thereof, comprises or consists of

(a) a VH comprising or consisting of an amino acid sequence of SEQ ID NO: 17, wherein X1 is D or E and X2 is selected from the group consisting of T, H, A, Y, N, E and S preferably in the group consisting of H, A, Y, N and E; and (b) a VL comprising or consisting of an amino acid sequence of SEQ ID NO: 26, wherein X is G or T.

More particularly, the invention relates to a bifunctional molecule comprises an anti-human PD-1 antibody or antigen-binding fragment thereof, that comprises or consists of:

(i) a heavy chain variable region (VH) comprising or consisting of an amino acid sequence of SEQ ID NO:24; and (ii) a light chain variable region (VL) comprising or consisting of an amino acid sequence of SEQ ID NO: 28.

Alternatively, the anti-PD1 antibody is selected from the group consisting of Pembrolizumab, Nivolumab, Pidilizumab, Cemiplimab, PDR001, and monoclonal antibodies 5C4, 17D8, 2D3, 4H1, 4A11, 7D3, and 5F4. In particular, the IL-7 or the variant thereof comprises or consists of an amino acid sequence having at least 75% identity with a wild type human IL-7 (wth-IL-7). In a particular aspect, the IL-7 comprises or consists of the amino acid sequence set forth in SEQ ID NO: 51.

Alternatively, the IL-7 is an IL-7 variant wherein the IL-7 variant presents at least 75% identity with a wild type human IL-7 (wth-IL-7) comprising or consisting of the amino acid sequence set forth in SEQ ID NO: 51, wherein the variant comprises at least one amino acid mutation which i) reduces affinity of the IL-7 variant for IL-7 receptor (IL-7R) in comparison to the affinity of wth-IL-7 for IL-7R, and ii) improves pharmacokinetics of the bifunctional molecule comprising the IL-7 variant in comparison with a bifunctional molecule comprising wth-IL-7.

In particular, the at least one mutation can be an amino acid substitution or a group of amino acid substitutions selected from the group consisting of (i) C2S-C141S and C47S-C92S, C2S-C141S and C34S-C129S, or C47S-C92S and C34S-C129S, (ii) W142H, W142F or W142Y, (iii) D74E, D74Q or D74N, iv) Q11E, Y12F, M17L, Q22E and/or K81R; or any combination thereof.

In one aspect, the IL-7 variant comprises a group of amino acid substitutions selected from the group consisting of C2S-C141S and C47S-C92S, C2S-C141S and C34S-C129S, and C47S-C92S and C34S-C129S.

In another aspect, the IL-7 variant comprises an amino acid substitution selected from the group consisting of W142H, W142F and W142Y.

In another aspect, the IL-7 variant comprises an amino acid substitution selected from the group consisting of D74E, D74Q and D74N.

Preferably, the IL-7 variant comprises or consists of the amino acid sequence set forth in SEQ ID NO: 53-66. Even more preferably, the IL-7 variant comprises or consists of the amino acid sequence set forth in SEQ ID NO: 54, 56 or 63.

In a particular aspect, the antibody or antigen-binding fragment thereof comprises a light chain constant domain derived from a human kappa light chain constant domain and a heavy chain constant domain derived from a human IgG1, IgG2, IgG3 or IgG4 heavy chain constant domain, preferably an IgG1 or IgG4 heavy chain constant domain.

In a more specific aspect, the antibody or antigen-binding fragment thereof comprises a light chain constant domain derived from a human kappa light chain constant domain and a heavy chain constant domain derived from a human IgG1 heavy chain constant domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/1332E; P257I/Q311; K326W/E333S; S239D/I332E/G236A; N297A; L234A/L235A; N297A+M252Y/S254T/T256E; K322A and K444A, preferably selected from the group consisting of N297A optionally in combination with M252Y/S254T/T256E, and L234A/L235A.

In another more specific aspect, the antibody or antigen-binding fragment thereof comprises a light chain constant domain derived from a human kappa light chain constant domain and a heavy chain constant domain derived from a human IgG4 heavy chain constant domain, optionally with a substitution or a combination of substitutions selected from the group consisting of S228P; L234A/L235A, S228P+M252Y/S254T/T256E and K444A.

Optionally, the antibody or a fragment thereof is linked to IL-7 or a variant thereof by a linker sequence, preferably selected from the group consisting of (GGGGS)₃, (GGGGS)₄, (GGGGS)₂, GGGGS, GGGS, GGG, GGS and (GGGS)₃, more preferably by (GGGGS)₃ or (GGGS)₃.

In a very specific aspect, the IL-7 variant comprises a group of amino acid substitutions selected from the group consisting of C2S-C141S and C47S-C92S, C2S-C141S and C34S-C129S, C47S-C92S and C34S-C129S, W142H, W142F, W142Y, D74E, D74Q and D74N; the antibody or antigen-binding fragment thereof comprises a light chain constant domain derived from a human kappa light chain constant domain and a heavy chain constant domain derived from a human IgG1 heavy chain constant domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/I332E; P257I/Q311; K326W/E333S; S239D/I332E/G236A; N297A; L234A/L235A; N297A+M252Y/S254T/T256E; K322A and K444A, preferably selected from the group consisting of N297A optionally in combination with M252Y/S254T/T256E, and L234A/L235A; and the antibody or a fragment thereof is linked to IL-7 variant by a linker (GGGGS)₃.

In another aspect, the invention concerns, an isolated nucleic acid sequence or a group of isolated nucleic acid molecules encoding a bifunctional molecule as disclosed herein, a vector comprising a nucleic acid or group of nucleic acid molecules as disclosed herein, and/or a host cell, comprising the vector the nucleic acid or group of nucleic acid molecules as disclosed herein.

In another aspect, the invention relates to a method for producing the bifunctional molecule, comprising a step of culturing a host cell according as disclosed herein and optionally a step of isolating the bifunctional molecule.

In another aspect, the invention concerns a pharmaceutical composition comprising the bifunctional molecule, the nucleic acid or group of nucleic acid molecules, the vector or the host cell as disclosed herein and a pharmaceutically acceptable carrier.

Optionally, the pharmaceutical composition further comprises an additional therapeutic agent, preferably selected in the group consisting of alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

Particularly, the pharmaceutical composition, bifunctional molecule, nucleic acid or group of nucleic acid molecules, vector or host cell are for use as a medicament.

The invention finally relates to a pharmaceutical composition, a bifunctional molecule, a nucleic acid or group of nucleic acid molecules, a vector, or a host cell as disclosed herein for use as a medicament, preferably for use in the treatment of cancer, preferably a cancer selected from the group consisting of a hematologic malignancy or a solid tumor with expression of PD-1 and/or PD-L1 such as a cancer selected from the group consisting of hematolymphoid neoplasms, angioimmunoblastic T cell lymphoma, myelodysplastic syndrome, and acute myeloid leukemia, a cancer induced by virus or associated with immunodeficiency such as a cancer selected from the group consisting of Kaposi sarcoma (e.g., associated with Kaposi sarcoma herpes virus); cervical, anal, penile and vulvar squamous cell cancer and oropharyngeal cancers (e.g., associated with human papilloma virus); B cell non-Hodgkin lymphomas (NHL) including diffuse large B-cell lymphoma, Burkitt lymphoma, plasmablastic lymphoma, primary central nervous system lymphoma, HHV-8 primary effusion lymphoma, classic Hodgkin lymphoma, and lymphoproliferative disorders (e.g., associated with Epstein-Barr virus (EBV) and/or Kaposi sarcoma herpes virus); hepatocellular carcinoma (e.g., associated with hepatitis B and/or C viruses); Merkel cell carcinoma (e.g., associated with Merkel cell polyoma virus (MPV)); and cancer associated with human immunodeficiency virus infection (HIV) infection, and a cancer selected from the group consisting of metastatic or not metastatic, Melanoma, malignant mesothelioma, Non-Small Cell Lung Cancer, Renal Cell Carcinoma, Hodgkin's Lymphoma, Head and Neck Cancer, Urothelial Carcinoma, Colorectal Cancer,

Hepatocellular Carcinoma, Small Cell Lung Cancer, Metastatic Merkel Cell Carcinoma, Gastric or Gastroesophageal cancers and Cervical Cancer.

Optionally, the bifunctional molecule, the pharmaceutical composition, the isolated nucleic acid molecule or the group of isolated nucleic acid molecules, the vector, or the host cell is for use in combination with radiotherapy or an additional therapeutic agent, preferably selected in the group consisting of alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (PIk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

The pharmaceutical composition, bifunctional molecule, nucleic acid or group of nucleic acid molecules, vector or host cell or uses as disclosed herein are for use for inhibiting of suppressive activity of T regulator cells, activating of T effector cells and/or stimulating proliferation of naive, partially exhausted and fully exhausted T-cells.

The pharmaceutical composition, bifunctional molecule, nucleic acid or group of nucleic acid molecules, vector or host cell or uses as disclosed herein can also be for use in the treatment of infectious disease, preferably chronic infectious disease, even more preferably chronic viral infections. Preferably, the infectious disease is caused by a virus selected from the group consisting of HIV, hepatitis virus, herpes virus, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

### BRIEF DESCRIPTION OF THE DRAWINGS

FIG. 1: PD-1 binding ELISA assay. Human recombinant PD-1 (rPD1) protein was immobilized and antibodies were added at different concentrations. Revelation was performed with an anti-human Fc antibody coupled to peroxidase. Colorimetry was determined at 450 nm using TMB substrate. A. Anti-PD1 (▪), anti-PD1VL-IL7 (∘) and anti-PD1VH-IL7 (●) were compared for their binding to recombinant PD1 (rPD1). B. Comparison of chimeric Bicki (●) versus humanized Bicki (▪)form of the anti-PD1 antibody fused to IL7 on its heavy or/and light chain: anti-PD1VH-IL7 (left graph), anti-PD1VL-IL7 (middle graph) or anti-PD1VHandVL-IL7 (right graph). Molecules were added at different concentrations on plate coated with human PD1 recombinant protein. C. PD-1 binding of Bicki anti PD-1/IL-7 constructed with Keytruda (●) and Opdivo (▪) Backbone were tested at different concentrations on plate coated with human PD1 recombinant protein.

FIG. 2: Antagonistic capacity of Bicki anti-PD1-IL7 molecule to block PD-1/PD-L1 and PD1/PDL2 interactions. A. ELISA assay: PD-L1 was immobilized on Maxisorp plate, the complex antibodies+biotinylated recombinant human PD-1 was added. This complex was generated with a fixed concentration of PD1 (0.6 μg/mL) and different concentrations of anti-PD1 (▪), anti-PD1VH-IL7 (●) or anti-PD1VL-IL7 (∘) antibodies were tested. B: Affinity assessment by Biacore of PD-1 recombinant protein on human PD-L2 recombinant protein pre-incubated with anti-PD1 antibody, anti-PD1VH-IL7 or anti-PD1VL-IL7 antibodies. Human recombinant PD-L2 is immobilized on the CM5 biochip and the complex antibody (200 nM)+recombinant human PD-1 (100 nM) was added. Data are represented in % of relative response of interaction as measured by Biacore: 100%=PD-1 relative response.

FIG. 3: Bicki anti-PD1-IL7 molecule stimulates IL-7R signaling pathway as measured by STAT5 phosphorylation ex vivo in human PBMCs. PBMCs isolated from peripheral blood of healthy volunteers were incubated 15 minutes with recombinant IL-7 (rIL7) (grey ●)+/−anti-PD1 (grey ∇), anti-PD1VH-IL7 (▪) or anti-PD1VL-IL7(●). Cells were then fixed, permeabilized and stained with an AF647 labeled anti-pSTAT5 (clone 47/Stat5(pY694)). Data were obtained by calculating MFI pSTAT* %pSTAT5+population and normalized (100%=rIL-7 (57.5 nM)) and represent the mean of 3 different donors in two independent experiments.

FIG. 4: Bicki anti-PD1-117 potentiates T cell activation in vitro. (A) Discover'x PD-1 Path Hunter Bioassay: Jurkat T cells stably expressing an engineered PD-1 receptor fused to Beta-gal fragment (ED) and an engineered SHP1 fused to complementing Beta-gal fragment (EA). The addition of anti-PD1 antagonist antibody blocks PD-1 signaling leading to a loss of bioluminescence signal (RLU). Anti-PD1 (▪) or anti-PD1VH-IL7 (●) were tested at different molar concentrations. Data are represented in RLU (Relative luminescence signal) (B) Promega PD-1/PD-L1 bioassay : (1) Effector T cells (Jurkat stably expressing PD-1, NFAT-induced luciferase) and (2) activating target cells (CHO K1 cells stably expressing PDL1 and surface protein designed to activate cognate TCRs in an antigen-independent manner) were co-cultured. After adding BIOGLO luciferin, luminescence is quantified and reflects T cell activation. Serial molar concentration of anti-PD1 antibody +/− recombinant IL-7 (rIL-7) or Bicki anti-PD1VH-IL7 or anti-PD1VL-IL7 antibodies were tested. Each dot represents EC50 of one experiment. (C) Capacity of Bicki anti PD-1/IL-7 constructed with Keytruda or Opdivo Backbone to stimulate NFAT. T cell activation was also tested using Promega PD-1/PD-L1 bioassay. Keytruda alone or Opdivo alone (●) versus Pembrolizumab VH IL-7 or Nivolumab VH IL-7(◯) were tested at different concentrations.

FIG. 5: Bicki anti PD1-IL7 molecule enhances IFNg secretion. T cells isolated from peripheral blood of healthy volunteers were stimulated with OKT3/PDL1 coated plate (2 and 5 μg/mL, respectively) in the presence of isotype control, anti-PD1+/−rIL7, anti-PD1VH-IL7, anti-PD1VL-IL7, isotypeVH-IL7 at a final antibody concentration of 5 μg/ml. Day 5 following stimulation, secreted IFNγ was dosed by sandwich ELISA. Result is representative of 4 donors (n=2 experiments).

FIG. 6: Bicki anti-PD1-IL7 enhances T cell proliferation to a similar extend to recombinant soluble IL-7. PBMCs cells isolated from peripheral blood of healthy volunteers were stimulated with anti-CD3/CD28. Twenty hours after stimulation, PBMCs were harvested and restimulated on OKT3/PDL1 coated plate (2 and 5 μg/mL, respectively) in the presence of rIL-7 or Bicki anti-PD1VH-IL7. (A) A fixed dose (29 nM of BiCKI or 3,2 nM rIL-7) or (B) Multiple doses of rIL-7 (□) anti-PD1 alone or anti-PD1VH-IL7(●) or anti-PD1VL-IL7 (▪) or isotype VH IL-7 (Δ) were tested. Day 5 following stimulation, T cell proliferation was assessed by 3H thymidine incorporation. Data were normalized (100%=rIL7 10 nM) and represent mean obtained on 3 different donors. EC50 (pM) refers to the concentration required to reach 50% of T cell proliferation.

FIG. 7. Bicki anti-PD1VH-IL7 stimulates expression of integrins on T cell surface. Human PBMCs were incubated 3 days without any molecules (grey histogram) or with rIL-7 /rIL-2 or rIL-7 (50 ng/mL) or anti-PD-1 or anti-PD1VH-IL7 (5 μg/mL) A. Alpha 4 and Beta 7 integrins cell surface expression analysis. FACS was analyzed by LSR and Data are represented in fold change normalized to 1 corresponding to the median of fluorescence of control (untreated) for each donor B. LFA-1 cell surface expression analysis (CD11a and CD18) by FACS using LSR. Results are expressed as the median fluorescence. Each dot represents one donor of 3 independent experiments.

FIG. 8. Modelisation of chronic antigen stimulation of T cells leading to exhausted T cells. Human PBMCs were repeatedly stimulated on CD3 CD28 coated plate (3 μg/mL of OKT3 and 3 μg/mL CD28.2 antibody), every 3 days. (A) Twenty-four hours following stimulation, T cells were stained for PD-1, Lag3 and Tim3 inhibitory receptors. Expression was analyzed by flow cytometry using fluorochrome labeled antibody and FACS LSRII. Data are represented in % of positive cells for 3 donors (one donor=one curve). (B) T cell proliferation capacity was determined by thymidine 3H incorporation, Day 5 following each stimulation. (C) Twenty-four hours following each stimulation, supernatant IFNg secretion was analyzed by ELISA (pg/ml).

FIG. 9. IL7 pathway activation of exhausted T cells: Response of exhausted T cells to 15 minutes incubation of cells with rIL-7 or Bicki anti-PD1VH-IL7 by measuring phosphorylation of STATS 48 h after each stimulation. Cells were then fixed, permeabilized and stained with a AF647 labeled anti-pSTATS (clone 47/Stat5 (pY694)). A. grey histograms represent cells treated with rIL7 and black histograms with Bicki anti-PD1VH-IL7. Data were normalized (MFI pSTAT*% pSTATS population) and are representative of 4 different donors. (B) ED50 of pSTATS was determined for each stimulation in pM and refers to the concentration of rIL-7 (▪ grey) or Bicki anti-PD1VH-IL7 (▪ black) required to reach 50% of pSTATS activation.

FIG. 10: Proliferation of exhausted T cells upon IL-7 stimulation. Human PBMCs were repeatedly stimulated on CD3 CD28 coated plate (3 μg/mL of OKT3 and 3 μg/mL CD28.2 antibody) (A) Twenty four hours after each stimulation, T cells were restimulated on OKT3 coated plate (2 μg/mL) in the presence of anti-PD1, rIL-7 or Bicki anti-PD1-IL7 (anti-PD1VH-IL7 or anti-PD1VL-IL7). H3 incorporation assay was performed on Day 5 to determine T cell proliferation. Raw proliferation data are shown (H3 incorporation (cpm)) from one donor after stimulation (STIM) 3, 4 and 5 (B) T cell stimulated 3 times were no more stimulated (No Stim) or restimulated on anti-CD3 (StimOKT3), anti-CD3+recombinant PDL1 (Stim OKT3/PDL1) or anti-CD3+recombinant PDL2 (StimOKT3/PDL2) coated plate (2 and 5 μg/mL respectively). H3 incorporation assay was performed on Day 5 and data were normalized (1=H3 incorporation with isotype Antibody). n=4 donors and 2 different experiments.

FIG. 11. Treg suppressive activity on proliferation of CD8 effector T cells. CD8+ effector T cells and CD4+1 CD25high CD127low Treg were isolated from peripheral blood of healthy donor, stained with cell proliferation dye (CPDe450 for CD8+ T cells). Treg/CD8+ Teff were then co-cultured at ratio 1:1 on OKT3 coated plate (2 μg/mL) in presence or absence of rIL-7, anti PD-1, anti-PD-1+rIL-7, anti-PD1VH-IL7 for 5 days. Proliferation of effector T cells was analyzed by cytofluorometry. A. Data represent % of proliferation Teff alone (black histogram) or Teff co-cultured with Treg (grey histogram) +/±SEM of based on loss of CPD Marker in CD8 T effector cell population. (n=4 donors in 4 different experiments). B. Treg proliferation was assessed using CPD proliferating Dye after incubation with different equimolar dose of IL-7 (▴), IL-2 (●), IL-15 (▪) or anti-PD1VH-IL7 (▾).

FIG. 12: In vivo efficacy of Bicki anti-PD1-IL7 antibody in a humanized mouse model. Human PBMCs were intraperitoneally injected into the mice. Mice were treated bi-weekly with anti-PD1 antibody alone or Bicki anti-PD1VH-IL7 (5 mg/kg). Day 16 after injection blood was harvested and mice sacrificed. (A) Percentage of peripheral human CD3 T cells was analyzed by flow cytometry in human CD45+ cell population. Each dot represents one mouse. (B) human IFNg was dosed in the plasma by ELISA. Each dot represents one mouse. (C) Infiltration of human CD3+ cells was quantified in the colon, the liver and lung by immunohistofluorescence. Proximal and distal colon, liver and lungs were embedded in Tissue Tek® OCT, and stained for Dapi and human CD3. Each dot represents one mouse and for the colon each dot represent mean of CD3+ count of 3 slices.

FIG. 13: Immunophenotyping of human tumor infiltrating lymphocytes. T cells were extracted from kidney cancer (Δ)(∇), metastatic colorectal (□), pancreatic cancer (∘) hepatocellular carcinoma (●)(⋄) and stained for CD3, CD4, CD8, PD-1, CD127 and CD132. Immunofluorescence was analyzed by FACS LSRII. Data are represented for CD4+CD3+ or CD8+CD3+ population.

FIG. 14: STAT5 Activation of intratumoral regulatory T cells or effector T cells in ex vivo tumors. Cells were extracted from tumor of schwannoma (▾), kidney (∘), hepatocellular carcinoma (□)(▪), metastatic colorectal (●) or pancreatic cancer (▴) patients and treated 15 minutes with rIL-7 or Bicki anti-PD1VH-IL7 (29 nM). Cells were then fixed, permeabilized and stained for pSTAT5 (clone 47/Stat5(pY694)), CD3 and Foxp3. Histograms represent pSTAT5 fluorescence median in CD3+ FoxP3+ population (T regulatory cells) or CD3+ FoxP3− (Effector T cells).

FIG. 15. In vitro study of IFNγ secretion after treatment of human cancer biopsies with Bicki anti-PD1-IL7: Biopsies of human tumor were mashed in a complete media to separate cells. Cells were resuspended in complete media with an isotype control, anti-PD1, B12-IL7 isotype control antibody (isotype-VH IL-7), anti PD-1 +recombinant IL-7, or with anti Bicki anti-PD1VH-IL7 at a concentration of 5 μg/mL. After 48 hours, supernatant was harvested and IFNγ secretion was analyzed using MSD technology (Meso scale Discovery). A. represents results on colorectal cancer cells and B. Results on individual tumors (CC: colorectal cancer biopsies, HCC: hepatocarcinoma biopsies, KC: Kidney cancers).

FIG. 16. STAT5 activation of intratumoral regulatory T cells or effector T cells after treatment with Bicki anti-PD1VH-IL7. (A) Percentage of intratumoral FoxP3 Treg cells into the tumor of colorectal cancer, schwannoma, kidney cancer or hepatocellular carcinoma (B) Cells from colorectal cancer (●), schwannoma (∘) and pancreatic cancer (□) were analyzed for STAT5 activation in FoxP3-CD3+ effector T cell versus FoxP3-CD3+ Treg cells after treatment with rIL7 or with anti-PD1VH-IL7 (29 nM) (15 min incubation). Cells were then fixed, permeabilized and stained for pSTAT5 (clone 47/Stat5(pY694)), CD3 and Foxp3.

FIG. 17: PD-1 binding ELISA assay of bicki IL-7 mutants. Human recombinant PD-1 (rPD1) protein was immobilized and antibodies were added at different concentrations. Revelation was performed with an anti-human Fc antibody coupled to peroxidase. Colorimetry was determined at 450 nm using TMB substrate. A. PD-1 binding of the bifunctional molecule comprising an anti-PD1 antibody and an IL-7 mutated on the amino acid D74, Q22, Y12F, M17, Q11, K81. B. PD-1 binding of the bifunctional molecule comprising an IL-7 mutated on the amino acid W142 C. PD-1 binding of the bifunctional molecule mutated in the disulfide bonds of IL-7 (SS1, SS2 and SS3 mutant). All molecules tested in this figure were constructed with an IgG4m isotype and a GGGGSGGGGSGGGGS linker between the Fc and IL-7 domain.

FIG. 18: CD127 binding ELISA assay of IgG fused mutated IL-7. PD-1 recombinant protein was immobilized on the plate, then bifunctional anti-PD-1 IL-7 molecules were preincubated with CD127 recombinant protein (Histidine tagged, Sino ref 10975-H08H) and added to the well. Revelation was performed with a mixture of an anti-histidine antibody coupled to biotin+streptavidin coupled to Peroxidase. Colorimetry was determined at 450 nm using TMB substrate. A. CD127 binding of the bifunctional molecule comprising IL-7 mutated on the amino acid D74, Q22, M17, Q11, Y12F, K81. B. CD127 binding of the bifunctional molecule comprising IL-7mutated on the amino acid W142.

FIG. 19: IL-7-7R signaling pathway of the different bifunctional molecules as measured by STAT5 phosphorylation. Human PBMCs isolated from peripheral blood of healthy volunteers were incubated 15 minutes with bifunctional anti-PD-1 IL-7 molecules. Cells were then fixed, permeabilized and stained with an AF647 labeled anti-pSTAT5 (clone 47/StatS(pY694)). Data were obtained by calculating MFI pSTAT5 in CD3 T cells. A. pSTAT5 activation of the anti-PD-1 IL-7 bifunctional molecule comprising an IL-7 mutated on the amino acid D74, Q22, M17, Y12F, Q11, K81. B. pSTAT activation of the anti PD-1 IL-7 bifunctional molecule comprising an IL-7 mutated on the amino acid W142 C. pSTAT5 activation of the anti PD-1 IL-7 bifunctional molecule comprising an IL-7 mutated in the disulfide bonds of IL-7 , SS2 (● black) and SS3 (▴) in comparison to anti PD-1 IL-7 WT (● grey). All molecules tested in this figure were constructed with an IgG4m isotype and a GGGGSGGGGSGGGGS linker between the Fc and IL-7 domain.

FIG. 20: Pharmacokinetics in mice of the anti PD-1 IL-7 bifunctional molecules Mice were intravenously injected with one dose with IgG fused IL-7 wild type or mutated IL-7. Concentration of the molecule in the sera was assessed by ELISA at multiple time points following injection. A. injection of IgG4-G4S3 IL7 WT (▪ grey); IgG4-G4S3 IL7 D74E (● black) B. injection of IgG4-G4S3 IL7 WT (▪ grey) or IgG4-GAS53 IL7 W142H (●black) C. injection of IgG4-G4S3 IL7 WT (▪ grey); IgG4-G4S3 IL7 SS2 (●) or IgG4-G4S3 IL7 SS3 (▴). D. Correlation between Area under the curve (AUC) calculated from PK vs ED50 pSTAT5 (nM) of each molecule. All molecules tested in this figure were constructed with an IgG4m isotype and a GGGGSGGGGSGGGGS linker between the Fc and IL-7 domain.

FIG. 21: The addition of a disulfide bond between anti PD-1 and IL-7 decreases pSTAT5 activation while it increases drug exposure in vivo. A. IL7R signaling as measured by pSTAT5 activation on human PBMCs after treatment with anti PD-1 IL-7 bifunctional molecule WT (grey ●) or anti PD-1 IL-7 bifunctional molecule with an additional disulfide bond(black ●) B. Pharmacokinetics in mice of the anti PD-1 IL-7 bifunctional molecule WT (grey ●) or anti PD-1 IL-7 bifunctional molecule with an additional disulfide bond (black ●) molecules. Mice were intravenously injected with one dose with ant PD-1 IL7 bifunctional molecules. Concentration of the molecule in the sera was assessed by ELISA at multiple time points following injection. All molecules tested in this figure were constructed with an IgG4m isotype and a GGGGSGGGGSGGGGS linker between the Fc and IL-7 domain.

FIG. 22: PD-1 binding ELISA assay. Human recombinant PD-1 (rPD1) protein was immobilized and antibodies were added at different concentrations. Revelation was performed with an anti-human Fc antibody coupled to peroxidase. Colorimetry was determined at 450 nm using TMB substrate. A. PD-1 binding of the anti PD-1 IL-7 WT bifunctional molecule with an IgG4m (● grey), anti PD-1 IL-7 WT bifunctional molecule with an IgG1m (▴ black), the anti PD-1 IL-7 D74E bifunctional molecule with an IgG1m isotype (▪) or anti PD-1 IL-7 W142H bifunctional molecule with an IgG1m (⋄). B. in another experiment, PD-1 binding of the anti PD-1 IL-7 SS2 bifunctional molecule with an IgG4m isotype (▪) or anti PD-1 IL-7 SS2 bifunctional molecule with an IgG1m (▴) were tested.

FIG. 23: CD127 binding ELISA assay of anti PD-1 IL-7 bifunctional molecule constructed with an IgG1N298A or IgG4 isotype. Recombinant protein targeted by the antibody backbone was immobilized, then antibodies fused to IL-7 were preincubated with CD127 recombinant protein (Histidine tagged, Sino ref 10975-H08H). Revelation was performed with a mixture of an anti-histidine antibody coupled to biotin and streptavidin coupled to Peroxidase. Colorimetry was determined at 450 nm using TMB substrate. A. CD127 binding of anti PD-1 IL-7 W142H bifunctional molecule with an IgG4m isotype (● grey), anti PD-1 IL-7 W142H bifunctional molecule with an IgG1m (▴ black), or the anti PD-1 IL-7 WT bifunctional molecule with an IgG1m isotype (● black). B. CD127 binding of the anti PD-1 IL-7 SS2 bifunctional molecule with an IgG4m isotype (● grey), anti PD-1 IL-7 SS2 bifunctional molecule with an IgG1m (▴ black) or the anti PD-1 IL-7 WT bifunctional molecule with an IgG1m (▴ black). C. CD127 binding of the anti PD-1 IL-7 SS3 bifunctional molecule with an IgG4m isotype (● grey), anti PD-1 IL-7 SS3 bifunctional molecule with an IgG1m (▴ black) or the anti PD-1 IL-7 WT bifunctional molecule IgG1m (● black) D. CD127 binding of the anti PD-1 W142H bifunctional molecule with an isotype IgG1m (● black) or an isotype IgG1m+YTE (● grey). The CD127 binding the anti PD-1 D74E bifunctional molecule with an isotype IgG1m (▴ black) or an isotype IgG1m +YTE (▴ grey) were also tested. All molecules tested in this figure were constructed with a GGGGSGGGGSGGGGS linker between the Fc and IL-7 domain.

FIG. 24: IL-7R signaling analysis of anti PD-1 IL-7 bifunctional molecule constructed with an IgG1N298A or IgG4 isotype. humans PBMCs or jurkat PD1+CD127+cells were incubated 15 minutes with anti PD-1 IL7 bifunctional molecule. Cells were then fixed, permeabilized and stained with an AF647 labeled anti-pSTAT5 (clone 47/Stat5(pY694)). Data were obtained by calculating % of pSTAT5 in CD3 T cells. A. pSTAT5 signaling on human PBMCs after treatment of the bifunctional molecule anti PD-1 IL-7 having the mutation D74E with an IgG4m isotype (● grey) or an IgG1m isotype (▴ black) B. pSTAT5 signaling on human PBMCs after treatment of the anti PD-1 IL-7 SS2 with an IgG4m isotype (● grey) or anti PD-1 IL-7 SS2 with an IgG1m (▴ black))C. pSTAT5 signaling on human PBMCs after treatment of the anti PD-1 IL-7 SS3 with an IgG4m isotype (● grey) or an IgG1m (▴ black) D. (left panel) pSTAT5 signaling on jurkat PD1+CD127+ cells after treatment of the anti PD-1 IL-7 WT constructed with an IgG4m (● grey) or IgG1m (▴ black) isotype D. (right panel) pSTAT5 signaling after treatment of the anti PD-1 IL-7 SS2 with an IgG4m isotype (● grey) or anti PD-1 IL-7 SS2 with an IgG1m (▴ black).

FIG. 25: Anti PD-1 IL-7 mutated bifunctional molecule potentiates T cell activation in vitro. Promega PD-1/PD-L1 bioassay: (1) Effector T cells (Jurkat stably expressing PD-1, NFAT-induced luciferase) and (2) activating target cells (CHO K1 cells stably expressing PDL1 and surface protein designed to activate cognate TCRs in an antigen-independent manner) were co-cultured. After adding BIOGLO luciferin, luminescence is quantified and reflects T cell activation. Serial molar concentration of anti-PD1 antibody +/− recombinant IL-7 (rIL-7) or anti-PD1IL7 bifunctional molecules were tested. Each dot represents EC50 of one experiment A. NFAT activation of the anti PD-1 IL-7 WT bifunctional molecule with an IgG4m isotype (● grey) or anti PD-1 (▴) or anti PD-1+rIL-7 (◯) B. NFAT activation of anti PD-1 IL-7 D74E IgG4m (●), PD-1 IL-7 D74E IgG1m (▴ dotted line), and anti PD-1 alone (black ▴). D. NFAT activation of anti PD-1 IL-7 W142H bifunctional molecule with IgG4m (●), PD-1 IL-7 W142H bifunctional molecule with IgG1m (▴ dotted line), and anti PD-1 alone (black ▴) D. NFAT activation of anti PD-1 IL-7 SS2 bifunctional molecule with IgG4m (●), and anti PD-1 alone (black ▴).

FIG. 26: Pharmacokinetics of anti PD-1 IL-7 bifunctional molecules constructed with an IgG1m or IgG4m isotype. Mice were intravenously injected with one dose with IgG fused to IL-7 wild type or to mutated IL-7. Concentration of the drug in the sera was assessed by ELISA at multiple time point following injection. A. Pharmacokinetics of the anti PD-1 IL-7 WT bifunctional molecule with IgG4m (● grey plain line), the anti PD-1 IL-7 WT bifunctional molecule with IgG1m (● grey dashed line), the anti PD-1 IL-7 D74E bifunctional molecule with IgG1m (● black dashed line), the anti PD-1 IL-7 W142H bifunctional molecule with IgG4m (◯ black plain line), the anti PD-1 IL-7 W142H bifunctional molecule with IgG1m (◯ dashed black plain line), the anti PD-1 IL-7 SS3 with IgG4 (▪ plain line), and the anti PD-1 IL-7 SS3 with IgG1m (▪ dashed line). B. Pharmacokinetics of anti PD-1 IL-7 D74E, D74Q, W142H, D74E+W142H mutant bifunctional molecules with an IgG1m.

FIG. 27: Pharmacokinetics of anti PD-1 IL-7 bifunctional molecule constructed with an IgG1 N298A+K444A isotype. Mice were intravenously injected with one dose anti PD-1 IL7 D74E bifunctional molecule with an isotype IgG1N298A (▪) or an isotype IgG1m+K444A mutation isotype (●). Concentration of the antibody was assessed by ELISA at multiple time point following injection.

FIG. 28: Length of the linker does not significantly impact pharmacokinetics but decreases the stimulation of IL-7R signaling. A. Pharmacokinetics of anti PD-1 IL-7 WT bifunctional molecules constructed with different linkers (GGGGS), (GGGGS)2, (GGGGS)3). B. Pharmacokinetics of anti PD-1 IL-7 D74 bifunctional molecules constructed with different linkers (GGGGS), (GGGGS)2, (GGGGS)3). C. Pharmacokinetics of anti PD-1 IL-7 W142H bifunctional molecules constructed with different linkers ((GGGGS)2, (GGGGS)3). Mice were intravenously injected with one dose with IgG fused to IL-7 wild type or mutated IL-7. Concentration of the IgG fused to IL-7 was assessed by ELISA at multiple time points following injection. D. pSTAT5 signaling of the anti PD-1 IL-7 bifunctional molecules constructed without linker or with GGGGS, (GGGGS)2, (GGGGS)3 linkers.

FIG. 29: the anti PD-1 IL-7 mutant preferentially target PD-1+ CD127+cells over PD-1-CD127+ cells. Jurkat cells expressing CD127+ or co-expressing CD127+and PD-1+were stained with 45nM of anti PD-1 IL-7 bifunctional molecule and revealed with an anti IgG-PE (Biolegend, clone HP6017). Data represent ratio of the Median fluorescence on PD-1+CD127+ Jurkat cells over the Median fluorescence obtained on PD1− cells CD127+ Jurkat cells. In this assay, anti PD-1 IL-7 WT bifunctional molecule IgG1m, anti PD-1 IL-7 D74E bifunctional molecule IgG1m, anti PD-1 IL-7 W142H bifunctional molecule IgG1m, anti PD-1 IL-7 SS2 bifunctional molecule IgG4m, anti PD-1 IL-7 SS3 bifunctional molecule IgG1m were tested.

FIG. 30: the anti PD-1 IL-7 molecule enhances proliferation of T cells and demonstrates preclinical safety in in cynomolgus monkeys. c target PD-1+ CD127+cells over PD-1−CD127+ cells. Cynomolgus monkeys were injected intravenously with one dose of bicki anti PD-1 IL-7 WT (6,87 nM/kg (n=2)) or 34,35 nM (n=1)). Blood analysis was performed until Day 15 or 4 hours following injection. A. lymphocyte count was assessed in the peripheral blood at multiple time points, Bicki anti PD-1 IL-7 WT injected at 6,87 nM/kg (n=2). B. proliferation of CD4/CD8 or Bcells was assessed by flow cytometry in the blood using Ki67 /CD4/CD8 and CD19 markers Bicki anti PD-1 IL-7 WT injected at 6,87 nM/kg (n=2). C. pSTAT5 was analyzed at multiple time points in CD3+ T cells by FACS Bicki anti PD-1 IL-7 WT injected at 6,87 nM/kg (n=2). D/E/F and G. Biochemical and cell blood analysis were assessed at multiple time points.

FIG. 31: Illustration of the mechanism of action of the Bicki anti-PD1-IL-7 according to the invention.

### DETAILED DESCRIPTION OF THE INVENTION

Introduction

The antibodies of the invention are bifunctional since they combine the specific anti-PD-1 effects and the effects of human interleukin 7 fused to the anti-PD-1 antibody. Indeed, the present invention relates to a bifunctional molecule comprising an anti-PD-1 antibody and IL-7, the interleukin being covalently linked to a polypeptide chain of the anti-PD-1 antibody, either the light chain or the heavy chain of the antibody or both or a fragment thereof. The chain of the anti-PD-1 antibody or a fragment thereof and the IL-7 are prepared as a fusion protein. In this particular aspect, the N terminal end of IL-7 is linked to the C terminal end of the chain of the anti-PD-1 antibody or a fragment thereof, optionally through a peptide linker. As known by the one skilled in the art, tumoral cells may not sufficiently be eliminated by T cells due to a phenomenon called T cells exhaustion, observed in many cancers. As described for instance by Jiang, Y., Li, Y. and Zhu, B (Cell Death Dis 6, e1792 (2015)), exhausted T cells in tumor microenvironment can lead to overexpression of inhibitory receptors, decrease of effector cytokine production and cytolytic activity, leading to the failure of cancer elimination and generally to cancer immune evasion. Restoring exhausted T cells is then a clinical strategy envisioned for cancer treatment.

PD-1 is the major inhibitory receptor regulating T-cell exhaustion. Indeed, T cells with high PD-1 expression have a decreased ability to eliminate cancer cells. Anti-PD1 therapeutic compounds, especially anti-PD1 antibody, are used clinically in the treatment of cancer for blocking the inhibiting effect of PD1-PDL1 interaction (PD1 on T cells and PDL1 on tumoral cells) and T cells exhaustion. However, anti-PD1 antibodies are not always sufficiently efficient to allow the « re »activation of exhausted T cells.

The applicant shows herein that the bifunctional anti-PD1-IL-7 molecules according to the invention potentiate activation (NFAT mediated activation) of T cells, in particular exhausted T cells, compared to anti PD-1 alone. Particularly, the anti-PD1-IL-7 bifunctional molecules induce the proliferation and activation of naive, partially exhausted and fully exhausted T-cell subsets as reflected by cytokine (e.g. IFNγ) secretion. Such anti-PD1-IL-7 bifunctional molecules have the capacity to overcome associated resistance mechanism and to improve efficacy of anti PD-1 immunotherapies.

Applicant particularly shows that the interaction of the anti-PD1-IL-7 bifunctional molecule, with a single T cell expressing i) PD1 and ii) IL-7 receptor, leads to unexpected activation of the NFAT pathway (TCR signaling) with a positive effect on T cells activation, in particular on exhausted T cells, favorizing the capacity of T cells to eliminate tumoral cells.

It means that, on one side, IL-7 of the bifunctional molecules of the present invention targets IL-7 receptors, activating PSTAT5 pathway, and on the other side, the anti-PD1 part of the bifunctional molecule blocks PD-1/PD-L1 interactions. The BICKI molecule targets both IL-7 to PD-1 on the same cell. This results in a synergistic activation of the TCR (NFAT) signaling, which has never been observed using a combination of anti-PD1 antibody and IL-7 separately (as two separate compounds). This activation cannot be provided by bifunctional molecule that targets PD-L1. Indeed, it is known in the art that PD-L1 is expressed on tumoral cells and not on immune cells such as T cells.

In addition, the bifunctional anti-PD1/IL-7 molecules allow accumulation of IL-7 in PD-1+ T cells infiltrates and re-localization of IL-7 on PD-1+ T cells. This accumulation of IL-7 near PD-1+ T cells is of particular interest in the context of exhausted T cells which require high dose of IL-7 for activating or re-activating these T cells.

The synergistic effect on the T cell activation has been observed not only with the particular anti-PD-1 antibody of the invention but also with two others anti-PD-1 of reference, namely Opdivo and Keytruda. In addition, the bifunctional anti-PD1/IL-7 molecule has the capacity to promote T-cell infiltration into the tumor. When considering that the lack of T cell infiltration in the tumor site is nowadays the major obstacle to efficacy of the treatment with anti-PD1 antibodies, this capacity is an advantage to optimize the treatment by anti-PD-1 antibodies.

Furthermore, the bifunctional anti-PD1/IL-7 molecules block Treg mediated inhibitory effect. Therefore, the bifunctional molecules are capable of specifically activate T effector cells and not the T reg cells whereas an anti-PD1 antibody is not able to inhibit Treg suppressive activity on T effector cells. Then, the inventors showed that the bifunctional anti-PD1-IL7 molecules favor the T cell effector over T regulatory immune balance by stimulating effector T-cell proliferation and survival while sparing regulatory T cells. Additionally, the IL7 anti-PD-1 bifunctional molecules have other advantages. The inventors show that IL7 anti-PD-1 bifunctional molecules activate predominantly T effector cells (Teff) versus T regulatory cells (Treg). The IL7 anti-PD-1 bifunctional molecule has the advantage of not promoting a proliferation of T Reg, of inducing an inactivation of T Reg, and of inducing an activation of T cells, in particular exhausted T cells.

Besides IL7 anti-PD-1 bifunctional molecules allow very advantageous dosages, exhibiting a favorable therapeutic index (the ratio between the letal dose (DL50) and the therapeutically efficient dose). In particular, IL7 can be used typically in the range 10 to 1500 μg/Kg for patients, advantageously 200-1200 μg/Kg; a high dose such as 1200 μg/Kg is well tolerated by patients. Then, IL7 anti-PD-1 bifunctional molecules allow to produce the therapeutic compound at an appropriate dosage of the raw compounds and of the final product. Indeed, the high dose well tolerated of IL7 (for instance around 1,2 mg/kg for IL7) corresponds to a quantity of about 2 mg/kg for the antibody, which is a satisfying dose to be administered to the patient.

IL7 anti-PD-1 bifunctional molecules have the advantage of allowing to essentially target exhausted T progenitor cells that are only partly exhausted, which are the key targets to meet the medical need mentioned above. It is added that chronical activation is important in case of viral infection which are associated to a similar exhaustion of T cells, therefore viral pathologies are included in the scope of the pathologies targeted by the new products of the application.

Finally, in a specific aspect, the inventors designed bifunctional molecules comprising IL-7 mutants or variants. The IL-7 mutants or variants are characterized by i) a reduced affinity for IL-7 receptor (IL-7R) in comparison to the affinity of wildtype IL-7, and ii) improves pharmacokinetics of the bifunctional molecule comprising the IL-7 variants in comparison with a bifunctional molecule comprising wildtype IL-7. Firstly, the use of IL-7 variants in the bifunctional molecules is important for increasing the pharmacokinetics in vivo of the bifunctional molecules. Secondly, by decreasing the affinity of IL-7 variants for its receptor, it increases the capacity of the bifunctional molecules to preferentially bind the targeted T cells by the anti-PD-1 antibody moiety of the bifunctional molecule and to present a specific effect on these cells but also to take advantage of the synergistic effect associated to the action of the two parts of the bifunctional molecule on the same T cells. The bifunctional molecules with an IL-7 variant have a good binding and antagonist activity of PD-1. In addition, the bifunctional molecules present a suitable equilibrium between its affinity to PD-1 and the affinity to IL-7R. Surprisingly, the inventors observed that the bifunctional molecules having an IgG1 heavy chain constant domain have an improved activity of IL-7 variants (pStat5 signal, synergistic effect and CD127 binding) compared to the same molecule with an IgG4 heavy chain constant domain. In addition, the use of a linker (GGGGS)₃ between the antibody and the IL-7 maximizes the activity of IL-7 variants (pStat5 signal and CD127 binding). The bifunctional molecules of the invention have in particular one or several of the following advantages:
The bifunctional molecules induce proliferation of naive, partially exhausted and fully exhausted T-cell subsets and not only partially exhausted T-cell as with anti-PD1/PDL1 therapy. More particularly, they have a synergistic effect of the T cells activation.
The bifunctional molecules allow a specific localization of IL-7 close or on PD-1+ exhausted T cells into the tumor, targeting cells that require higher concentration of IL-7. They particularly induce the accumulation of IL-7 in PD-1+T cells infiltrates and re-localization of IL-7 on PD-1+T cells.
Whereas anti-PD-1 blockades fail to reprogram exhausted T cells into active memory T cells, limiting the long-term clearance of the tumor, the bifunctional molecules promote formation, survival and proliferation of memory T cells via the presence of IL-7. Then, the bifunctional molecules induce a durable anti-tumor immunity through sustained and expanded memory T cell response.
Whereas anti-PD1/PD-L1 therapy efficacy is associated with pre-existing T cell infiltration and T cell effector functions, in particular IFNγ signature, the bifunctional molecules increase the proliferation of effector T cells and their capacity to secrete IFNγ, with a synergistic effect.
The bifunctional molecules may decrease the immunosuppressive microenvironment by decreasing T reg population and inhibiting secretion of TGFβ (a suppressive cytokine). More particularly, the bifunctional molecules specifically stimulate the effector T cells without stimulating the T reg.
The bifunctional molecule in which IL-7 could be fused to the C-terminal part of the Heavy and/or light chain and where IL7 conserves a high affinity for CD127, similar to naked/natural IL-7. The bifunctional molecule may be more potent in term of IL-7R activation and half-life.
They are produced with high production yield as bifunctional molecules.
The bifunctional molecule decreases the immunosuppressive activity of Treg cells into Tumor microenvironment by decreasing the number of T reg. More particularly, the bifunctional molecule specifically stimulates the effector T cells without stimulating the T reg.
The bifunctional compounds increase the expression of integrins (i.e., Alpha4 and/or Beta7 and LFAT) promoting T cell infiltration into the tissues and/or tumors compare to anti-PD1 response only. In particular, the bifunctional compounds promote T cells migration and tumor infiltration.
The bifunctional molecules may comprise IL-7 variants or mutants as identified by the inventors in order to maximize the pharmacokinetics in vivo while maintaining the IL-7 activity and the antagonist activity of the anti-DP-1 antibody, with a suitable affinity balance between IL-7 and IL-7R and anti-PD-1 and PD-1.

Definitions

In order that the present invention may be more readily understood, certain terms are defined hereafter.

Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

As used herein, the terms “interleukin-7”, “IL-7” and “IL-7” refers to a mammalian endogenous secretory glycoprotein, particularly IL-7 polypeptides, derivatives and analogs thereof having substantial amino acid sequence identity to wild-type mammalian IL-7 and substantially equivalent biological activity, e.g., in standard bioassays or assays of IL-7 receptor binding affinity. For example, IL-7 refers to an amino acid sequence of a recombinant or non-recombinant polypeptide having an amino acid sequence of: i) a native or naturally-occurring allelic variant of an IL-7 polypeptide, ii) a biologically active fragment of an IL-7 polypeptide, iii) a biologically active polypeptide analog of an IL-7 polypeptide, or iv) a biologically active variant of an IL-7 polypeptide. The IL-7 can comprise its peptide signal or be devoid of it. Alternative designations for this molecule are “pre-B cell growth factor” and “Iymphopoietin-1”. Preferably, the term “IL-7” refers to human IL-7. For example, the human IL-7 amino acid sequence is about 152 amino acids (in absence of signal peptide) and has a Genbank accession number of NP_000871.1, the gene being located on chromosome 8q12-13. Human IL-7 is described in UniProtKB-P13232.

As used herein, the terms “wild type interleukin-7”, “wt-IL-7” and “wt-IL7” refers to a mammalian endogenous secretory glycoprotein, particularly IL-7 polypeptides, derivatives and analogs thereof having substantial amino acid sequence identity to wild-type functional mammalian IL-7 and substantially equivalent biological activity, e.g., in standard bioassays or assays of IL-7 receptor binding affinity. For example, wt-IL-7 refers to an amino acid sequence of a recombinant or non-recombinant polypeptide having an amino acid sequence of: i) a native or naturally-occurring IL-7 polypeptide, ii) a biologically active fragment of an IL-7 polypeptide, iii) a biologically active polypeptide analog of an IL-7 polypeptide, or iv) a biologically active IL-7 polypeptide. The IL-7wt can comprise its peptide signal or be devoid of it. Alternative designations for this molecule are “pre-B cell growth factor” and “Iymphopoietin-1”. Preferably, the term “wt-IL-7” refers to human IL-7 (wth-IL7). For example, the human wt-IL-7 amino acid sequence is about 152 amino acids (in absence of signal peptide) and has a Genbank accession number of NP_000871.1, the gene being located on chromosome 8q12-13. Human IL-7 is for example described in UniProtKB-P13232.

As used herein, the terms “Programmed Death 1”, “Programmed Cell Death 1”, “PD1”, “PD-1”, “PDCD1”, “PD-1 antigen”, “human PD-1”, “hPD-1” and “hPD-1” are used interchangeably and refer to the Programmed Death-1 receptor, also known as CD279, and include variants and isoforms of human PD-1, and analogs having at least one common epitope with PD-1. PD-1 is a key regulator of the threshold of immune response and peripheral immune tolerance. It is expressed on activated T cells, B cells, monocytes, and dendritic cells and binds to its ligands PD-L1 and PD-L2. Human PD-1 is encoded by the PDCD1 gene. As an example, the amino acid sequence of a human PD-1 is disclosed under GenBank accession number NP_005009. PD1 has four splice variants expressed on human Peripheral blood mononuclear cells (PBMC). Accordingly, PD-1 proteins include full-length PD-1, as well as alternative splice variants of PD- 1, such as PD-1Aex2, PD-1Aex3, PD-1Aex2,3 and PD-1Aex2,3,4. Unless specified otherwise, the terms include any variant and isoform of human PD-1 that are naturally expressed by PBMC, or that are expressed by cells transfected with a PD-1 gene.

As used herein, the term “antibody” describes a type of immunoglobulin molecule and is used in its broadest sense. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. The heavy-chain constant domains that correspond to the different classes of immunoglobulins are called alpha, delta, epsilon, gamma, and mu, respectively. Unless specifically noted otherwise, the term “antibody” includes intact immunoglobulins and “antibody fragment” or “antigen binding fragment” (such as Fab, Fab′, F(ab′)2, Fv), single chain (scFv), mutants thereof, molecules comprising an antibody portion, diabodies, linear antibodies, single chain antibodies, and any other modified configuration of the immunoglobulin molecule that comprises an antigen recognition site of the required specificity, including glycosylation variants of antibodies, amino acid sequence variants of antibodies. Preferably, the term antibody refers to a humanized antibody.

As used herein, an “antigen-binding fragment” of an antibody means a part of an antibody, i.e. a molecule corresponding to a portion of the structure of the antibody of the invention, that exhibits antigen-binding capacity for PD-1, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. Antigen-binding fragments of antibodies are fragments which comprise their hypervariable domains designated CDRs (Complementary Determining Regions) or part(s) thereof encompassing the recognition site for the antigen, i.e. the extracellular domain of PD1, thereby defining antigen recognition specificity.

A “Fab” fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)2 pepsin digestion product. Additional chemical couplings of antibody fragments are known to those of ordinary skill in the art. Fab and F(ab′)2 fragments lack the Fc fragment of an intact antibody, clear more rapidly from the circulation of animals, and may have less non-specific tissue binding than an intact antibody (see, e.g. Wahl et al, 1983, J. Nucl. Med. 24:316).

An “Fv” fragment is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target, although at a lower affinity than the entire binding site.

“Single-chain Fv” or “scFv” antibody binding fragments comprise the VH and VL domains of an antibody, where these domains are present in a single polypeptide chain. Generally, the Fv polypeptide further comprises a polypeptide linker between the VH and VL domains which enables the scFv to form the desired structure for target binding.

“Single domain antibodies” are composed of a single VH or VL domains which exhibit sufficient affinity to PD-1. In a specific embodiment, the single domain antibody is a camelized antibody {See, e.g., Riechmann, 1999, Journal of Immunological Methods 231 :25-38).

In terms of structure, an antibody may have heavy (H) chains and light (L) chains interconnected by disulfide bonds. There are two types of light chain, lambda (λ) and kappa (κ). Each heavy and light chain contains a constant region and a variable region (or “domain”). Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, and U.S. Department of Health and Human Services, 1991, which is hereby incorporated by reference). Preferably, the CDRs are defined according to Kabat method. The framework regions act to form a scaffold that provides, for positioning the CDRs in correct orientation by inter-chain, non-covalent interactions. The CDRs are primarily responsible for binding to an epitope of an antigen. The CDRs of each chain are typically referred to as “Complementarity Determining Region 1” or “CDR1”, “CDR2”, and “CDR3”, numbered sequentially starting from the N-terminus. The VL and VH domain of the antibody according to the invention may comprise four framework regions or “FR's”, which are referred to in the art and herein as “Framework region 1 ” or “FR1”, “FR2”, “FR3”, and “FR4”, respectively. These framework regions and complementary determining regions are preferably operably linked in the following order: FR1-CDR1-FR2-CDR2-FR3-CDR3-FR4 (from amino terminus to carboxy terminus). The term “antibody framework” as used herein refers to the part of the variable domain, either VL and/or VH, which serves as a scaffold for the antigen binding loops (CDRs) of this variable domain.

An “antibody heavy chain” as used herein, refers to the larger of the two types of polypeptide chains present in antibody conformations. The CDRs of the antibody heavy chain are typically referred to as “HCDR1”, “HCDR2” and “HCDR3”. The framework regions of the antibody heavy chain are typically referred to as “HFR1”, “HFR2”, “HFR3” and “HFR4”.

An “antibody light chain,” as used herein, refers to the smaller of the two types of polypeptide chains present in antibody conformations; κ and λ light chains refer to the two major antibody light chain isotypes. The CDRs of the antibody light chain are typically referred to as “LCDR1”, “LCDR2” and “LCDR3”.

The framework regions of the antibody light chain are typically referred to as “LFR1”, “LFR2”, “LFR3” and “LFR4”.

With regard to the binding of an antibody to a target molecule, the terms “bind” or “binding” refer to peptides, polypeptides, proteins, fusion proteins, molecules and antibodies (including antibody fragments) that recognize and contact an antigen. Preferably, it refers to an antigen-antibody type interaction. The terms “specific binding”, “specifically binds to,” “specific for,” “selectively binds” and “selective for” a particular antigen (e.g., PD-1) or an epitope on a particular antigen (e.g., PD-1) mean that the antibody recognizes and binds a specific antigen, but does not substantially recognize or bind other molecules in a sample. For example, an antibody that specifically (or preferentially) binds to PD-1 or to a PD-1 epitope is an antibody that binds this PD-1 epitope for example with greater affinity, avidity, more readily, and/or with greater duration than it binds to other PD-1 epitopes or non-PD-1 epitopes. Preferably, the term “specific binding” means the contact between an antibody and an antigen with a binding affinity equal or lower than 10⁻⁷ M. In certain aspects, antibodies bind with affinities equal or lower than 10⁻⁸ M, 10⁻⁹ M or 10⁻¹⁰ M.

As used herein “PD-1 antibody,” “anti-PD-1 antibody,” “PD-1 Ab,” “PD-1-specific antibody”, “anti-PD-1 Ab” are used interchangeably and refer to an antibody, as described herein, which specifically binds to PD-1, particularly human PD-1. In some embodiments, the antibody binds to the extracellular domain of PD-1. Particularly, an anti-PD-1 antibody is an antibody capable of binding to a PD-1 antigen and inhibits the PD-1-mediated signaling pathway, thereby enhancing immune responses such as T cell activation. As used herein, the term “bifunctional molecule”, “bifunctional compound”, “bifunctional protein”, “Bicki”, “Bicki antibody”, “bifunctional antibody” and “bifunctional checkpoint inhibitors molecule” have the same meanings and can be interchangeably used. These terms refer to an antibody that recognizes one antigen by virtue of possessing at least one region (e.g. derived from a variable region of an antibody) that is specific for this antigen, and at least a second region that is a polypeptide. More specifically, the bifunctional molecule is a fusion protein of an antibody or a portion thereof, preferably an antigen binding fragment thereof with another polypeptide or polypeptide fragment thereof.

The term “chimeric antibody” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human species, such as from a mouse.

As used herein, the term “humanized antibody” is intended to refer to antibodies in which CDR sequences derived from the germline of another mammalian species, such as a mouse, have been grafted onto human framework sequences (e.g. chimeric antibodies that contain minimal sequence derived from a non-human antibody). A “humanized antibody”, e.g., a non-human antibody, also refers to an antibody that has undergone humanization. A humanized antibody is generally a human immunoglobulin (recipient antibody) in which residues from one or more CDRs are replaced by residues from at least one CDR of a non-human antibody (donor antibody) while maintaining the desired specificity, affinity, and capacity of the original antibody. The donor antibody can be any suitable non-human antibody, such as a mouse, rat, rabbit, chicken, or non-human primate antibody having a desired specificity, affinity, or biological effect.

In some instances, selected framework region residues of the recipient antibody are replaced by framework region residues from the donor antibody. Alternatively, selected framework region residues of the donor antibody are replaced by framework region residues from a human or humanized antibody. Additional framework region modifications may be made within the human framework sequences. Humanized antibodies thus may also comprise residues that are not found in either the recipient antibody or the donor antibody. Such amino acid modifications may be made to further refine antibody function and/or increased the humanization process. By “amino acid change” or “amino acid modification” is meant herein a change in the amino acid sequence of a polypeptide. “Amino acid modifications” include substitution, insertion and/or deletion in a polypeptide sequence. By “amino acid substitution” or “substitution” herein is meant the replacement of an amino acid at a particular position in a parent polypeptide sequence with another amino acid. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). As used herein, “amino acid position” or “amino acid position number” are used interchangeably and refer to the position of a particular amino acid in an amino acids sequence, generally specified with the one letter codes for the amino acids. The first amino acid in the amino acids sequence (i.e. starting from the N terminus) should be considered as having position 1.

A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

TABLE A
Amino Acid Residue

| Amino Acid groups | Amino Acid Residues |
|---|---|
| Acidic Residues | ASP and GLU |
| Basic Residues | LYS, ARG, and HIS |
| Hydrophilic Uncharged Residues | SER, THR, ASN, andGLN |
| Aliphatic Uncharged Residues | GLY, ALA, VAL, LEU, and ILE |
| Non-polar Uncharged Residues | CYS, MET, and PRO |
| Aromatic Residues | PHE, TYR, andTRP |

TABLE B
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C
Further Alternative Physical and Functional Classifications of Amino
Acid Residues

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, W, and Y |
| Negatively charged residues | D and E |
| Polar residues | C, D, E, H, K, N, Q, R, S, and T |
| Small residues | A, C, D, G, N, P, S, T, and V |
| Very small residues | A, G, and S |
| Residues involved in turn | A, C, D, E, G, H, K, N, Q, R, S, P, and T |
| formation | |
| Flexible residues | E, Q, T, K, S, G, P, D, E, and R |

As used herein, an “isolated antibody” is an antibody that has been separated and/or recovered from a component of its natural environment. An isolated antibody includes an antibody in situ within recombinant cells, since at least one component of the antibody's natural environment is not present. In some embodiments, an antibody is purified to homogeneity and/or to greater than 90%, 95% or 99% purity as determined by, for example, electrophoretic (e.g., SDS-PAGE, isoelectric focusing (IEF), capillary electrophoresis) or chromatographic (e.g., ion exchange or reverse phase HPLC) under reducing or non-reducing conditions.

The terms “derive from” and “derived from” as used herein refers to a compound having a structure derived from the structure of a parent compound or protein and whose structure is sufficiently similar to those disclosed herein and based upon that similarity, would be expected by one skilled in the art to exhibit the same or similar properties, activities and utilities as the claimed compounds. For example, a humanized antibody derived from a murine antibody refers to an antibody or antibody fragment that shares similar properties with the murine antibody, e.g. recognizes the same epitope, shares similar VH and VL with modified residues that participate and/or increased the humanization of the antibody. The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such a term refers to the improvement or eradication of a disease, a disorder, an infection or symptoms associated with it. In other embodiments, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect. Preferably, the term “treatment” refers to the application or administration of a composition including one or more active agents to a subject, who has a disorder/disease, for instance associated with the signaling pathway mediated by PD-1.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavorable environmental factors. Preferably, these terms refer to a health disorder or disease e.g. an illness that disrupts normal physical or mental functions. More preferably, the term disorder refers to immune and/or inflammatory diseases that affect animals and/or humans, such as cancer.

The term “immune disease”, as used herein, refers to a condition in a subject characterized by cellular, tissue and/or organ injury caused by an immunologic reaction of the subject to its own cells, tissues and/or organs. The term “inflammatory disease” refers to a condition in a subject characterized by inflammation, e.g., chronic inflammation. Autoimmune disorders may or may not be associated with inflammation. Moreover, inflammation may or may not be caused by an autoimmune disorder.

The term “cancer” as used herein is defined as disease characterized by the rapid and uncontrolled growth of aberrant cells. Cancer cells can spread locally or through the bloodstream and lymphatic system to other parts of the body.

As used herein, the term “disease associated with or related to PD-1”, “PD-1 positive cancer” or “PD-1 positive infectious disease” is intended to refer to the cancer or infectious disease (e.g. caused by a virus and/or bacteria) which is resulted from PD-1 expression or has the symptom/characteristic of PD-1 expression, i.e. any condition that is caused by, exacerbated by, or otherwise linked to increased or decreased expression or activities of PD-1.

As used herein, the term “subject”, “host”, “individual,” or “patient” refers to human, including adult and child.

As used herein, a “pharmaceutical composition” refers to a preparation of one or more of the active agents, such as comprising a bifunctional molecule according to the invention, with optional other chemical components such as physiologically suitable carriers and excipients. The purpose of a pharmaceutical composition is to facilitate administration of the active agent to an organism. Compositions of the present invention can be in a form suitable for any conventional route of administration or use. In one embodiment, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

“An effective amount” or a “therapeutic effective amount” as used herein refers to the amount of active agent required to confer therapeutic effect on the subject, either alone or in combination with one or more other active agents, e.g. the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

As used herein, the term “medicament” refers to any substance or composition with curative or preventive properties against disorders or diseases.

The term “in combination” as used herein refers to the use of more than one therapy (e.g., prophylactic and/or therapeutic agents). The use of the term “in combination” does not restrict the order in which therapies (e.g., prophylactic and/or therapeutic agents) are administered to a subject with a disease or disorder.

The terms “polynucleotide”, “nucleic acid” and “nucleic acid sequence” are equivalent and refer to a polymeric form of nucleotide of any length, for example RNA or DNA or analogs thereof. Nucleic acids (e.g., components, or portions, of the nucleic acids) of the present invention may be naturally occurring, modified or engineered, isolated and/or non-natural. Engineered nucleic acids include recombinant nucleic acids and synthetic nucleic acids.

“Isolated nucleic acid encoding an anti-PD1 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell. As used herein, the terms “nucleic acid construct”, “plasmid”, and “vector” are equivalent and refer to a nucleic acid molecule that serves to transfer a passenger nucleic acid sequence, such as DNA or RNA, into a host cell.

As used herein, the term “host cell” is intended to include any individual cell or cell culture that can be or has been recipient of vectors, exogenous nucleic acid molecules, and polynucleotides encoding the antibody construct of the present invention; and/or recipients of the antibody construct itself. The introduction of the respective material into the cell can be carried out by way of transformation, transfection and the like. The term “host cell” is also intended to include progeny or potential progeny of a single cell. Host cells include for example bacterial, microbial, plant and animal cells.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT) and myeloid-derived cells (neutrophil, eosinophil, basophil, monocyte, macrophage, dendritic cells). In particular, the immune cell can be selected in the non-exhaustive list comprising B cells, T cells, in particular CD4+ T cells and CD8+ T cells, NK cells, NKT cells, APC cells, dendritic cells and monocytes. “T cell” as used herein includes for example CD4+T cells, CD8+T cells, T helper 1 type T cells, T helper 2 type T cells, T helper 17 type T cells and inhibitory T cells.

As used herein, the term “T effector cell”, “T eff” or “effector cell” describes a group of immune cells that includes several T cells types that actively respond to a stimulus, such as co-stimulation. It particularly includes T cells which function to eliminate antigen (e.g., by producing cytokines which modulate the activation of other cells or by cytotoxic activity). It notably includes CD4+, CD8+, Treg cells, cytotoxic T cells and helper T cells (Th1 and Th2).

As used herein, the term “regulatory T cell”, “Treg cells” or “T reg” refers to a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FOXP3, and CD25 and are thought to be derived from the same lineage as naive CD4 cells.

The term “exhausted T cell” refers to a population of T cell in a state of dysfunction (i.e. “exhaustion”). T cell exhaustion is characterized by progressive loss of function, changes in transcriptional profiles and sustained expression of inhibitory receptors. Exhausted T cells lose their cytokines production capacity, their high proliferative capacity and their cytotoxic potential, which eventually leads to their deletion. Exhausted T cells typically indicate higher levels of CD43, CD69 and inhibitory receptors combined with lower expression of CD62L and CD127.

The term “immune response” refers to the action of, for example, lymphocytes, antigen presenting cells, phagocytic cells, granulocytes, and soluble macromolecules produced by the above cells or the liver (including antibodies, cytokines, and complements) that results in selective damage to, destruction of, or elimination from the human body of invading pathogens, cells or tissues infected with pathogens, cancerous cells, or, in cases of autoimmunity or pathological inflammation, normal human cells or tissues. The term “antagonist” as used herein, refers to a substance that block or reduces the activity or functionality of another substance. Particularly, this term refers to an antibody that binds to a cellular receptor (e.g. PD-1) as a reference substance (e.g. PD-L1 and/or PD-L2), preventing it from producing all or part of its usual biological effects (e.g. the creation of an immune suppressive microenvironment). The antagonist activity of an antibody according to the invention may be assessed by competitive ELISA. As used herein, the term “isolated” indicates that the recited material (e.g., antibody, polypeptide, nucleic acid, etc.) is substantially separated from, or enriched relative to, other materials with which it occurs in nature. Particularly, an “isolated” antibody is one which has been identified and separated and/or recovered from a component of its natural environment. For example, the isolated antibody is purified (1) to greater than 75% by weight of antibody as determined by the Lowry method, or (2) to homogeneity by SDS-PAGE under reducing or non-reducing conditions. Isolated antibody includes the antibody in situ within recombinant cells since at least one component of the antibody's natural environment will not be present. Ordinarily, however, isolated antibody will be prepared by at least one purification step.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to +/−10% (e.g., +/−0.5%, +/−1%, +/−1.5%, +/−2%, +/−2.5%, +/−3%, +/−3.5%, +/−4%, +/−4.5%, +/−5%, +/−5.5%, +/−6%, +/−6.5%, +/−7%, +/−7.5%, +/−8%, +/−8.5%, +/−9%, +/−9.5%). The use of the term “about” at the beginning of a string of values modifies each of the values (i.e. “about 1, 2 and 3” refers to about 1, about 2 and about 3). Further, when a listing of values is described herein (e.g. about 50%, 60%, 70%, 80%, 85% or 86%) the listing includes all intermediate and fractional values thereof (e.g., 54%, 85.4%).

Anti-PD-1 Antibody

The bifunctional molecule according to the invention comprises a first entity that comprises an anti-hPD-1 antibody or an antigen binding fragment thereof.

Provided herein are antibodies that particularly bind to human PD-1. In some aspects, the antibody specifically binds to human PD-1, preferably to the extracellular domain of human PD-1. In some aspects, the antibody selectively binds to one or more of full-length human PD-1, PD-1Aex2, PD-1Aex3, PD-1Aex2,3 and PD-1Aex2,3,4.

In some aspects, the anti-PD1 antibody is an isolated antibody, particularly a non-natural isolated antibody. Such isolated anti-PD1 antibody can be prepared by at least one purification step. In some embodiments, an isolated anti-PD1 antibody is purified to at least 80%, 85%, 90%, 95% or 99% by weight.

In some embodiments, an isolated anti-PD1 isolated antibody is provided as a solution comprising at least 85%, 90%, 95%, 98%, 99% to 100% by weight of an antibody, the remainder of the weight comprising the weight of other solutes dissolved in the solvent.

Preferably, such antibody has the ability to block or inhibit the interaction between PD-1 and at least one of its ligand (e.g. PD-L1 and/or PD-L2). The ability to “block binding” or “block interaction” or “inhibit interaction” as used herein refers to the ability of an antibody or antigen-binding fragment to prevent the binding interaction between two molecules (e.g. PD-1 and its ligand PD-L1 and/or PD-L2) to any detectable degree.

Preferably, the anti-PD1 antibody or antigen binding fragment thereof is an antagonist of the binding of human PD-L1 and/or PD-L2 to human PD-1, more preferably of human PD-L1 and PD-L2 to human PD-1.

In certain embodiments, the anti-hPD1 antibody or antigen-binding fragment inhibits the binding interaction between PD-1 and at least one of its ligands (e.g. PD-L1 and/or PD-L2, preferably PD-L1 and PD-L2) by at least 50%. In certain embodiments, this inhibition may be greater than 60%, greater than 70%, greater than 80%, or greater than 90%.

Anti-hPD1 antibodies according to this invention may comprise immunoglobulins, immunoglobulin of any class, such as IgD, IgE, IgG, IgA, or IgM (or sub-class thereof), immunoglobulin chains or fragments thereof (such as Fv, Fab, Fab', F(ab')2, scFv or other antigen-binding subsequences of antibodies) which contain minimal sequence derived from a non-human (e.g. murine) immunoglobulin targeting human PD-1. Preferably, the anti-hPD-1 antibody according to the invention derives from IgG1, IgG2, IgG3 or IgG4, preferably from an IgG4 or an IgG1.

In one embodiment, the antigen-binding fragment of an antibody comprises a heavy chain comprising a heavy chain variable domain comprising HCDR1, HCDR2 and HCDR3 and a light chain comprising a variable domain comprising LCDR1, LCDR2 and LCDR3, and a fragment of a heavy chain constant domain. By a fragment of a heavy chain constant domain, it should be understood that the antigen-binding fragment therefore comprises at least a portion of a full heavy chain constant domain. As examples, a heavy chain constant domain may comprise or consist of at least the C_{H}1 domain of a heavy chain, or at least the C_{H}1 and the C_{H}2 domains of a heavy chain, or at least the C_{H}1, C_{H}2 and C_{H}3 domains of a heavy chain. A fragment of a heavy chain constant domain may also be defined as comprising at least a portion of the Fc domain of the heavy chain. Accordingly, antigen-binding fragment of an antibody encompasses the Fab portion of a full antibody, the F(ab′)₂ portion of a full antibody, the Fab' portion of a full antibody. The heavy chain constant domain may also comprise or consist in a full heavy chain constant domain, for example illustrated in the present description, wherein several full heavy chain constant domains are described. In a particular embodiment of the invention, and when the antigen-binding fragment of an antibody comprises a fragment of a heavy chain constant domain comprising or consisting in a portion of a full heavy chain constant domain, the heavy chain constant domain fragment may consist of at least 10 amino acid residues; or may consist of 10 to 300 amino acid residues, in particular 210 amino acid residues.

Preferably, the antibody against human PD-1 is a monoclonal antibody. The term “monoclonal antibody” as used herein refers to an antibody obtained from a population of substantially homogeneous antibodies, i.e., the individual antibodies comprising the population are identical and/or bind the same epitope.

Preferably, such monoclonal antibodies (mAbs) are from a mammalian, such as mice, rodents, rabbit, goat, primates, non-human primates or humans. Techniques for preparing such monoclonal antibodies may be found in, e.g., Stites et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y.

In certain embodiments, the anti-hPD1 antibody provided herein is a chimeric antibody. In one example, the chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, the anti-hPD1 antibody is a humanized antibody. A humanized antibody typically comprises one or more variable domains in which CDRs (or portions thereof) are derived from a non-human antibody, and FRs (or portions thereof) are derived from human or humanized antibody sequences. Alternatively, some FR residues can be substituted to restore or improve antibody specificity, affinity and/or humanization. A humanized antibody optionally will also comprise at least a portion of a human or humanized constant region (Fc). Methods of antibodies humanization are well known in the art see for example, Winter and Milstein, Nature, 1991, 349:293-299; Riechmann et al., Nature, 332, pp. 323 (1988); Verhoeyen et al., Science, 239, pp. 1534 (1988), Rader et al, Proc. Nat. Acad. Sci. U.S.A., 1998, 95:8910-8915; Steinberger et al, J. Biol. Chem., 2000, 275:36073-36078; Queen et al, Proc. Natl. Acad. Sci. U.S.A., 1989, 86: 10029-10033; Almagro, J. C. and Fransson, J., Front. Biosci. 13 (2008) 1619-1633; Kashmiri, S. V. et al, Methods 36 (2005) 25-34 (describing SDR (a-CDR) grafting); Padlan, E. A., Mol. Immunol. 28 (1991) 489-498 (describing “resurfacing”); Dall'Acqua, W. F. et al, Methods 36 (2005) 43-60 (describing “FR shuffling”); and Osbourn, J. et al, Methods 36 (2005) 61-68 and Klimka, A. et al, Br. J. Cancer 83 (2000) 252-260 (describing the “guided selection” approach to FR shuffling) and U.S. Pat. Nos. 5,585,089, 5,693,761, 5,693,762, 5,821,337, 7,527,791, 6,982,321, and 7,087,409; and 6,180,370. Preferably, the humanized antibody against human PD-1 is a monoclonal antibody.

Particularly, a humanized antibody is one that has a T20 humanness score of at least 80% or at least 85%, more preferably at least 88%, even more preferably at least 90%, most preferably a T20 humanness score comprised between 85% and 95%, preferably between 88% and 92%.

“Humanness” is generally measured using the T20 score analyzer to quantify the humanness of the variable region of monoclonal antibodies as described in Gao S H, Huang K, Tu H, Adler A S. BMC Biotechnology. 2013: 13:55. T20 humanness score is a parameter commonly used in the field of antibody humanization first disclosed by Gao et al (BMC Biotechnol., 2013, 13, 55). T20 humanness score is usually used in patent application for defining a humanized antibody (e.g., WO15161311, WO17127664, WO18136626, WO18190719, WO19060750, or WO19170677).

A web-based tool is provided to calculate the T20 score of antibody sequences using the T20 Cutoff Human Databases: http://abAnalyzer.lakepharma.com. In computing a T20 score, an input VH, VK, or VL variable region protein sequence is first assigned Kabat numbering, and CDR residues are identified. The full-length sequence or the framework only sequence (with CDR residues removed) is compared to every sequence in a respective antibody database using the blastp protein-protein BLAST algorithm. The sequence identity between each pairwise comparison is isolated, and after every sequence in the database has been analyzed, the sequences are sorted from high to low based on the sequence identity to the input sequence. The percent identity of the Top 20 matched sequences is averaged to obtain the T20 score. For each chain type (VH, VK, VL) and sequence length (full-length or framework only) in the “All Human Databases,” each antibody sequence was scored with its respective database using the T20 score analyzer.

The T20 score was obtained for the top 20 matched sequences after the input sequence itself was excluded (the percent identity of sequences 2 through 21 were averaged since sequence 1 was always the input antibody itself). The T20 scores for each group were sorted from high to low. The decrease in score was roughly linear for most of the sequences; however the T20 scores for the bottom ˜15% of antibodies started decreasing sharply. Therefore, the bottom 15 percent of sequences were removed and the remaining sequences formed the T20 Cutoff Human Databases, where the T20 score cutoff indicates the lowest T20 score of a sequence in the new database.

Accordingly, the humanized anti-PD1 antibody comprised in the bifunctional molecule according to the invention has a T20 humanness score of at least 80% or at least 85%, more preferably at least 88%, even more preferably at least 90%, most preferably a T20 humanness score comprised between 85% and 95%, preferably between 88% and 92%.

In one embodiment, the anti-PD1 antibody can be selected from the group consisting of Pembrolizumab (also known as Keytruda lambrolizumab, MK-3475), Nivolumab (Opdivo, MDX-1106, BMS-936558, ONO-4538), Pidilizumab (CT-011), Cemiplimab (Libtayo), Camrelizumab, AUNP12, AMP-224, AGEN-2034, BGB-A317 (Tisleizumab), PDR001 (spartalizumab), MK-3477, SCH-900475, PF-06801591, JNJ-63723283, genolimzumab (CBT-501), LZM-009, BCD-100, SHR-1201, BAT-1306, AK-103 (HX-008), MEDI-0680 (also known as AMP-514) MEDI0608, JS001 (see Si-Yang Liu et al., J. Hematol. Onco1.10:136 (2017)), BI-754091, CBT-501, INCSHR1210 (also known as SHR-1210), TSR-042 (also known as ANB011), GLS-010 (also known as WBP3055), AM-0001 (Armo), STI-1110 (see WO 2014/194302), AGEN2034 (see WO 2017/040790), MGA012 (see WO 2017/19846), or 1131308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168. Bifunctional or bispecific molecules targeting PD-1 are also known such as RG7769 (Roche), XmAb20717 (Xencor), MED15752 (AstraZeneca), FS118 (F-star), SL-279252 (Takeda) and XmAb23104 (Xencor).

In a particular embodiment, the anti-PD1 antibody can be Pembrolizumab (also known as Keytruda lambrolizumab, MK-3475) or Nivolumab (Opdivo, MDX-1106, BMS-936558, ONO-4538).

A particular example of a humanized anti-hPD1 antibody is described hereafter by its CDRs, framework regions and Fc and hinge region.

CDR

“Complementarity determining regions” or “CDRs” are known in the art as referring to non-contiguous sequences of amino acids within antibody variable regions, which confer antigen specificity and binding affinity. The precise amino acid sequence boundaries of a given CDR can be readily determined using any of a number of well-known schemes, including those described by Kabat et al., (Sequences of Proteins of Immunological Interest 5th ed. (1991) “Kabat” numbering scheme); Al-Lazikani et al., 1997, J. Mol. Biol, 273:927-948 (“Chothia” numbering scheme); MacCallum et al, 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol, 2001, 309:657-70 (“AHo” numbering scheme). Unless otherwise specified, the numbering scheme used for identification of a particular CDR herein is the Kabat numbering scheme.

In one embodiment, the bifunctional molecule comprises a humanized anti-hPD-1 antibody or an antigen binding fragment thereof. The CDRs regions of the humanized antibody may be derived from a murine antibody and have been optimized to i) provide a safe humanized antibody with a very high level of humanization (superior to 85%) and stability ; and ii) increase the antibody properties, more particularly a higher manufacturability when produced in mammalian cells and a higher production yield in mammal cells such as COS and HCO cells while preserving an antagonist activity (i.e. inhibition of the binding of human PD-L1 to human PD-1), as they have a binding affinity (KD) for a human PD-1 less than 10⁻⁷ M, preferably less than 10⁻⁸ M.

In a very particular embodiment, the bifunctional molecule comprises an anti-human-PD-1 antibody or antigen binding fragment thereof, preferably a humanized anti-human-PD-1 antibody or antigen binding fragment thereof that comprises:

(i) a heavy chain variable domain comprising HCDR1, HCDR2 and HCDR3, and

(ii) a light chain variable domain comprising LCDR1, LCDR2 and LCDR3,

wherein:

the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;

the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2;

the heavy chain CDR3 (HCDR3) comprises or consists of an amino acid sequence of SEQ ID NO: 3 wherein X1 is D or E and X2 is selected from the group consisting of T, H, A, Y, N, E and S, preferably in the group consisting of H, A, Y, N, E; optionally with one, two or three modification(s) selected from substitution(s), addition(s), deletion(s) and any combination thereof at any position but positions 2, 3, 7 and 8 of SEQ ID NO: 3;

the light chain CDR1 (LCDR1) comprises or consists of an amino acid sequence of SEQ ID NO: 12 wherein X is G or T, optionally with one, two or three modification(s) selected from substitution(s), addition(s), deletion(s) and any combination thereof at any position but positions 5, 6, 10, 11 and 16 of SEQ ID NO: 12;

the light chain CDR2 (LCDR2) comprises or consists of an amino acid sequence of SEQ ID NO: 15, optionally with one, two or three modification(s) selected from substitution(s), addition(s), deletion(s) and any combination thereof; and

the light chain CDR3 (LCDR3) comprises or consists of an amino acid sequence of SEQ ID NO:16, optionally with one, two or three modification(s) selected from substitution(s), addition(s), deletion(s) and any combination thereof at any position but positions 1, 4 and 6 of SEQ ID NO: 16.

In one aspect, the bifunctional molecule comprises a humanized anti-hPD-1 antibody or an antigen binding fragment thereof that comprises:

(i) a heavy chain variable domain comprising HCDR1, HCDR2 and HCDR3, and

(ii) a light chain variable domain comprising LCDR1, LCDR2 and LCDR3,

wherein:

the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;

the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2;

the heavy chain CDR3 (HCDR3) comprises or consists of an amino acid sequence of SEQ ID NO: 3 wherein either X1 is D and X2 is selected from the group consisting of T, H, A, Y, N, E, and S preferably in the group consisting of H, A, Y, N, E; or X1 is E and X2 is selected from the group consisting of T, H, A, Y, N, E and S, preferably in the group consisting of H, A, Y, N, E and S; optionally with one, two or three modification(s) selected from substitution(s), addition(s), deletion(s) and any combination thereof at any position but positions 2, 3, 7 and 8 of SEQ ID NO: 3;

the light chain CDR1 (LCDR1) comprises or consists of an amino acid sequence of SEQ ID NO: 12 wherein X is G or T, optionally with one, two or three modification(s) selected from substitution(s), addition(s), deletion(s) and any combination thereof at any position but positions 5, 6, 10, 11 and 16 of SEQ ID NO: 12;

the light chain CDR2 (LCDR2) comprises or consists of an amino acid sequence of SEQ ID NO: 15, optionally with one, two or three modification(s) selected from substitution(s), addition(s), deletion(s) and any combination thereof; and

the light chain CDR3 (LCDR3) comprises or consists of an amino acid sequence of SEQ ID NO: 16, optionally with one, two or three modification(s) selected from substitution(s), addition(s), deletion(s) and any combination thereof at any position but positions 1, 4 and 6 of SEQ ID NO: 16.

In another embodiment, the bifunctional molecule comprises a humanized anti-hPD-1 antibody or an antigen binding fragment thereof that comprises:

(i) a heavy chain variable domain comprising HCDR1, HCDR2 and HCDR3, and

(ii) a light chain variable domain comprising LCDR1, LCDR2 and LCDR3,

wherein:

the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;

the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2;

the heavy chain CDR3 (HCDR3) comprises or consists of an amino acid sequence of SEQ ID NO: 4, 5, 6, 7, 8, 9, 10 or 11 optionally with one, two or three modification(s) selected from substitution(s), addition(s), deletion(s) and any combination thereof at any position but positions 2, 3, 7 and 8 of SEQ ID NO: 4, 5, 6, 7, 8, 9, 10 or 11;

the light chain CDR1 (LCDR1) comprises or consists of an amino acid sequence of SEQ ID NO: 13 or SEQ ID NO:14, optionally with one, two or three modification(s) selected from substitution(s), addition(s), deletion(s) and any combination thereof at any position but positions 5, 6, 10, 11 and 16 of SEQ ID NO: 13 or SEQ ID NO: 14;

the light chain CDR2 (LCDR2) comprises or consists of an amino acid sequence of SEQ ID NO: 15, optionally with one, two or three modification(s) selected from substitution(s), addition(s), deletion(s) and any combination thereof; and

the light chain CDR3 (LCDR3) comprises or consists of an amino acid sequence of SEQ ID NO:16, optionally with one, two or three modification(s) selected from substitution(s), addition(s), deletion(s) and any combination thereof at any position but positions 1, 4 and 6 of SEQ ID NO: 16.

In another aspect, the bifunctional molecule comprises a humanized anti-hPD-1 antibody or an antigen binding fragment thereof that comprises:

(i) a heavy chain variable domain comprising HCDR1, HCDR2 and HCDR3, and

(ii) a light chain variable domain comprising LCDR1, LCDR2 and LCDR3,

wherein:

(a) the light chain CDR1 (LCDR1) comprises or consists of an amino acid sequence of SEQ ID NO: 13, optionally with one, two or three modification(s) selected from substitution(s), addition(s), deletion(s) and any combination thereof at any position but positions 5, 6, 10, 11 and 16 of SEQ ID NO: 13;

(b) the light chain CDR2 (LCDR2) comprises or consists of an amino acid sequence of SEQ ID NO: 15, optionally with one, two or three modification(s) selected from substitution(s), addition(s), deletion(s) and any combination thereof;

(c) the light chain CDR3 (LCDR3) comprises or consists of an amino acid sequence of SEQ ID NO:16, optionally with one, two or three modification(s) selected from substitution(s), addition(s), deletion(s) and any combination thereof at any position but positions 1, 4 and 6 of SEQ ID NO: 16;

(d) the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;

(e) the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2; and

(f) the heavy chain CDR3 (HCDR3) comprises or consists of an amino acid sequence of SEQ ID NO: 4, optionally with one, two or three modification(s) selected from substitution(s), addition(s), deletion(s) and any combination thereof at any position but positions 2, 3, 7 and 8 of SEQ ID NO: 4; or

the heavy chain CDR3 (HCDR3) comprises or consists of an amino acid sequence of SEQ ID NO: 5, optionally with one, two or three modification(s) selected from substitution(s), addition(s), deletion(s) and any combination thereof at any position but positions 2, 3, 7 and 8 of SEQ ID NO: 5; or

the heavy chain CDR3 (HCDR3) comprises or consists of an amino acid sequence of SEQ ID NO: 6, optionally with one, two or three modification(s) selected from substitution(s), addition(s), deletion(s) and any combination thereof at any position but positions 2, 3, 7 and 8 of SEQ ID NO: 6; or

the heavy chain CDR3 (HCDR3) comprises or consists of an amino acid sequence of SEQ ID NO: 7, optionally with one, two or three modification(s) selected from substitution(s), addition(s), deletion(s) and any combination thereof at any position but positions 2, 3, 7 and 8 of SEQ ID NO:7; or

the heavy chain CDR3 (HCDR3) comprises or consists of an amino acid sequence of SEQ ID NO: 8 optionally with one, two or three modification(s) selected from substitution(s), addition(s), deletion(s) and any combination thereof at any position but positions 2, 3, 7 and 8 of SEQ ID NO: 8; or

the heavy chain CDR3 (HCDR3) comprises or consists of an amino acid sequence of SEQ ID NO: 9 optionally with one, two or three modification(s) selected from substitution(s), addition(s), deletion(s) and any combination thereof at any position but positions 2, 3, 7 and 8 of SEQ ID NO: 9; or

the heavy chain CDR3 (HCDR3) comprises or consists of an amino acid sequence of SEQ ID NO: 10 optionally with one, two or three modification(s) selected from substitution(s), addition(s), deletion(s) and any combination thereof at any position but positions 2, 3, 7 and 8 of SEQ ID NO: 10; or

the heavy chain CDR3 (HCDR3) comprises or consists of an amino acid sequence of SEQ ID NO: 11 optionally with one, two or three modification(s) selected from substitution(s), addition(s), deletion(s) and any combination thereof at any position but positions 2, 3, 7 and 8 of SEQ ID NO: 11.

In another aspect, the bifunctional molecule comprises a humanized anti-hPD-1 antibody or an antigen binding fragment thereof that comprises:

(i) a heavy chain variable domain comprising HCDR1, HCDR2 and HCDR3, and

(ii) a light chain variable domain comprising LCDR1, LCDR2 and LCDR3,

wherein:

(a) the light chain CDR1 (LCDR1) comprises or consists of an amino acid sequence of SEQ ID NO: 14, optionally with one, two or three modification(s) selected from substitution(s), addition(s), deletion(s) and any combination thereof at any position but positions 5, 6, 10, 11 and 16 of SEQ ID NO: 14; (b) the light chain CDR2 (LCDR2) comprises or consists of an amino acid sequence of SEQ ID NO: 15, optionally with one, two or three modification(s) selected from substitution(s), addition(s), deletion(s) and any combination thereof;

(c) the light chain CDR3 (LCDR3) comprises or consists of an amino acid sequence of SEQ ID NO:16, optionally with one, two or three modification(s) selected from substitution(s), addition(s), deletion(s) and any combination thereof at any position but positions 1, 4 and 6 of SEQ ID NO: 16;

(d) the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;

(e) the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2; and

(f) the heavy chain CDR3 (HCDR3) comprises or consists of an amino acid sequence of SEQ ID NO: 4, optionally with one, two or three modification(s) selected from substitution(s), addition(s), deletion(s) and any combination thereof at any position but positions 2, 3, 7 and 8 of SEQ ID NO: 4; or

the heavy chain CDR3 (HCDR3) comprises or consists of an amino acid sequence of SEQ ID NO: 5, optionally with one, two or three modification(s) selected from substitution(s), addition(s), deletion(s) and any combination thereof at any position but positions 2, 3, 7 and 8 of SEQ ID NO: 5; or

the heavy chain CDR3 (HCDR3) comprises or consists of an amino acid sequence of SEQ ID NO: 6, optionally with one, two or three modification(s) selected from substitution(s), addition(s), deletion(s) and any combination thereof at any position but positions 2, 3, 7 and 8 of SEQ ID NO: 6; or

the heavy chain CDR3 (HCDR3) comprises or consists of an amino acid sequence of SEQ ID NO: 7, optionally with one, two or three modification(s) selected from substitution(s), addition(s), deletion(s) and any combination thereof at any position but positions 2, 3, 7 and 8 of SEQ ID NO:7; or

the heavy chain CDR3 (HCDR3) comprises or consists of an amino acid sequence of SEQ ID NO: 8 optionally with one, two or three modification(s) selected from substitution(s), addition(s), deletion(s) and any combination thereof at any position but positions 2, 3, 7 and 8 of SEQ ID NO: 8; or

the heavy chain CDR3 (HCDR3) comprises or consists of an amino acid sequence of SEQ ID NO: 9 optionally with one, two or three modification(s) selected from substitution(s), addition(s), deletion(s) and any combination thereof at any position but positions 2, 3, 7 and 8 of SEQ ID NO: 9; or

the heavy chain CDR3 (HCDR3) comprises or consists of an amino acid sequence of SEQ ID NO: 10 optionally with one, two or three modification(s) selected from substitution(s), addition(s), deletion(s) and any combination thereof at any position but positions 2, 3, 7 and 8 of SEQ ID NO: 10; or

the heavy chain CDR3 (HCDR3) comprises or consists of an amino acid sequence of SEQ ID NO: 11 optionally with one, two or three modification(s) selected from substitution(s), addition(s), deletion(s) and any combination thereof at any position but positions 2, 3, 7 and 8 of SEQ ID NO: 11.

In a particular aspect, the modifications are substitutions, in particular conservative substitutions.

In one embodiment, the anti-human-PD-1 antibody or antigen binding fragment thereof comprises (i) a heavy chain comprising a CDR1 of SEQ ID NO: 1, a CDR2 of SEQ ID NO: 2 and a CDR3 of SEQ ID NO: 3 wherein X1 is D or E and X2 is selected from the group consisting of T, H, A, Y, N, E and S, preferably in the group consisting of H, A, Y, N and E; and (ii) a light chain comprising a CDR1 of SEQ ID NO: 12 wherein X is G or T, a CDR2 of SEQ ID NO: 15 and a CDR3 of SEQ ID NO: 16.

In one embodiment, the anti-human-PD-1 antibody or antigen binding fragment thereof comprises (i) a heavy chain comprising a CDR1 of SEQ ID NO: 1, a CDR2 of SEQ ID NO: 2 and a CDR3 of SEQ ID NO: 3 wherein X1 is D and X2 is selected from the group consisting of T, H, A, Y, N and E, preferably in the group consisting of H, A, Y, N and E; or wherein X1 is E and X2 is selected from the group consisting of T, H, A, Y, N, E, and S, preferably in the group consisting of H, A, Y, N, E and S; and (ii) a light chain comprising a CDR1 of SEQ ID NO: 12 wherein X is G or T, a CDR2 of SEQ ID NO: 15 and a CDR3 of SEQ ID NO: 16.

In one embodiment, the anti-human-PD-1 antibody or antigen binding fragment thereof comprises (i) a heavy chain comprising a CDR1 of SEQ ID NO: 1, a CDR2 of SEQ ID NO: 2 and a CDR3 of SEQ ID NO: 3 wherein X1 is D and X2 is selected from the group consisting of T, H, A, Y, N and E, preferably in the group consisting of H, A, Y, N and E; and (ii) a light chain comprising a CDR1 of SEQ ID NO: 12 wherein X is G or T, a CDR2 of SEQ ID NO: 15 and a CDR3 of SEQ ID NO: 16.

In one embodiment, the anti-human-PD-1 antibody or antigen binding fragment thereof comprises (i) a heavy chain comprising a CDR1 of SEQ ID NO: 1, a CDR2 of SEQ ID NO: 2 and a CDR3 of SEQ ID NO: 3 wherein X1 is E and X2 is selected from the group consisting of T, H, A, Y, N, E, and S, preferably in the group consisting of H, A, Y, N, E and 5; and (ii) a light chain comprising a CDR1 of SEQ ID NO: 12 wherein X is G or T, a CDR2 of SEQ ID NO: 15 and a CDR3 of SEQ ID NO: 16. In another embodiment, the anti-human-PD-1 antibody or antigen binding fragment thereof comprises or consists essentially of (i) a heavy chain comprising a CDR1 of SEQ ID NO: 1, a CDR2 of SEQ ID NO: 2 and a CDR3 of SEQ ID NO: 4, 5, 6, 7, 8, 9, 10 or 11; and (ii) a light chain comprising a CDR1 of SEQ ID NO: 13 or SEQ ID NO:14, a CDR2 of SEQ ID NO: 15 and a CDR3 of SEQ ID NO: 16.

In another embodiment, the anti-human-PD-1 antibody or antigen binding fragment thereof comprises or consists essentially of

(i) a heavy chain comprising a CDR1 of SEQ ID NO: 1, a CDR2 of SEQ ID NO: 2 and a CDR3 of SEQ ID NO: 4; and (ii) a light chain comprising a CDR1 of SEQ ID NO: 13, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 5; and (ii) a light chain comprising a CDR1 of SEQ ID NO: 13, a CDR2 of SEQ ID NO: 15 and a CDR3 of SEQ ID NO: 16, or

(i) a heavy chain comprising a CDR1 of SEQ ID NO: 1, a CDR2 of SEQ ID NO: 2 and a CDR3 of SEQ ID NO: 6; and (ii) a light chain comprising a CDR1 of SEQ ID NO: 13, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 7; and (ii) a light chain comprising a CDR1 of SEQ ID NO: 13, a CDR2 of SEQ ID NO: 15 and a CDR3 of SEQ ID

(i) a heavy chain comprising a CDR1 of SEQ ID NO: 1, a CDR2 of SEQ ID NO: 2 and a CDR3 of SEQ ID NO: 8; and (ii) a light chain comprising a CDR1 of SEQ ID NO: 13, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 9; and (ii) a light chain comprising a CDR1 of SEQ ID NO: 13, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 10; and (ii) a light chain comprising a CDR1 of SEQ ID NO: 13, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 11; and (ii) a light chain comprising a CDR1 of SEQ ID NO: 13, a CDR2 of SEQ ID NO: 15 and a CDR3 of SEQ ID NO: 16.

In another embodiment, the anti-human-PD-1 antibody or antigen binding fragment thereof comprises or consists essentially of

(i) a heavy chain comprising a CDR1 of SEQ ID NO: 1, a CDR2 of SEQ ID NO: 2 and a CDR3 of SEQ ID NO: 4; and (ii) a light chain comprising a CDR1 of SEQ ID NO: 14, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 5; and (ii) a light chain comprising a CDR1 of SEQ ID NO: 14, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 6; and (ii) a light chain comprising a CDR1 of SEQ ID NO: 14, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 7; and (ii) a light chain comprising a CDR1 of SEQ ID NO: 14, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 8; and (ii) a light chain comprising a CDR1 of SEQ ID NO: 14, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 9; and (ii) a light chain comprising a CDR1 of SEQ ID NO: 14, a CDR2 of SEQ ID NO: 15 and a CDR3 of SEQ ID

(i) a heavy chain comprising a CDR1 of SEQ ID NO: 1, a CDR2 of SEQ ID NO: 2 and a CDR3 of SEQ ID NO: 10; and (ii) a light chain comprising a CDR1 of SEQ ID NO: 14, a CDR2 of SEQ ID NO: 15 and a CDR3 of SEQ ID NO: 16; or

(i) a heavy chain comprising a CDR1 of SEQ ID NO: 1, a CDR2 of SEQ ID NO: 2 and a CDR3 of SEQ ID NO: 11; and (ii) a light chain comprising a CDR1 of SEQ ID NO: 14, a CDR2 of SEQ ID NO: 15 and a CDR3 of SEQ ID NO: 16.

Framework

In one embodiment, the anti-PD1 antibody or antigen binding fragment according to the invention comprises framework regions, in particular heavy chain variable region framework regions (HFR) HFR1, HFR2, HFR3 and HFR4 and light chain variable region framework regions (LFR) LFR1, LFR2, LFR3 and LFR4. Preferably, the anti-PD1 antibody or antigen binding fragment according to the invention comprises human or humanized framework regions. A “human acceptor framework” for the purposes herein is a framework comprising the amino acid sequence of a light chain variable domain (VL) framework or a heavy chain variable domain (VH) framework derived from a human immunoglobulin framework or a human consensus framework, as defined below. A human acceptor framework derived from a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence. A “human consensus framework” is a framework which represents the most commonly occurring amino acid residues in a selection of human immunoglobulin VL or VH framework sequences.

Particularly, the anti-PD1 antibody or antigen binding fragment comprises heavy chain variable region framework regions (HFR) HFR1, HFR2, HFR3 and HFR4 comprising an amino acid sequence of SEQ ID NOs: 41, 42, 43 and 44, respectively, optionally with one, two or three modification(s) selected from substitution(s), addition(s), deletion(s) and any combination thereof at any position but positions 27, 29 and 32 of HFR3, i.e., of SEQ ID NO: 43. Preferably, the anti-PD1 antibody or antigen binding fragment comprises HFR1 of SEQ ID NO: 41, HFR2 of SEQ ID NO: 42, HFR3 of SEQ ID NO: 43 and HFR4 of SEQ ID NO: 44.

Alternatively or additionally, the anti-PD1 antibody or antigen binding fragment comprises light chain variable region framework regions (LFR) LFR1, LFR2, LFR3 and LFR4 comprising an amino acid sequence of SEQ ID NOs: 45, 46, 47 and 48, respectively, optionally with one, two or three modification(s) selected from substitution(s), addition(s), deletion(s) and any combination thereof. Preferably, the humanized anti-PD1 antibody or antigen binding fragment comprises LFR1 of SEQ ID NO: 45, LFR2 of SEQ ID NO: 46, LFR3 of SEQ ID NO: 47 and LFR4 of SEQ ID NO: 48.

VH-VL

The VL and VH domain of the anti hPD1 antibody comprised in the bifunctional molecule according to the invention may comprise four framework regions interrupted by three complementary determining regions preferably operably linked in the following order: FR1-CDR1-FR2-CDR2-FR3-CDR3-FR4 (from amino terminus to carboxy terminus).

In a first embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;

(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In a second embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 17, wherein either X1 is D and X2 is selected from the group consisting of T, H, A, Y, N, E, preferably in the group consisting of H, A, Y, N, E; or X1 is E and X2 is selected from the group consisting of T, H, A, Y, N, E and S preferably in the group consisting of H, A, Y, N, E and 5; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;

(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In a third embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 17, wherein X1 is D and X2 is selected from the group consisting of T, H, A, Y, N, E, preferably in the group consisting of H, A, Y, N, E, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;

(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In another embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 17, wherein X1 is E and X2 is selected from the group consisting of T, H, A, Y, N, E and S preferably in the group consisting of H, A, Y, N, E and 5; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;

(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In another embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 18, 19, 20, 21, 22, 23, 24 or 25, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18, 19, 20, 21, 22, 23, 24 or 25, respectively;

(b) a light chain variable region (VL) comprising or consisting of an amino acid sequence of SEQ ID NO: 27 or SEQ ID NO: 28, optionally with one, two or three modification(s) selected from substitution(s), addition(s), deletion(s) and any combination thereof at any position positions 3, 4, 7, 14, 17, 18, 28, 29, 33, 34, 39, 42, 44, 50, 81, 88, 94, 97, 99 and 105 of SEQ ID NO: 27 or SEQ ID NO: 28.

In another embodiment, the anti-human-PD-1 humanized antibody or antigen binding fragment thereof comprised in the bifunctional molecule comprises:

(a) a heavy chain variable region (VH) comprising or consisting of an amino acid sequence of SEQ ID NO: 18 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or

(a) a heavy chain variable region (VH) comprising or consisting of an amino acid sequence of SEQ ID NO: 19 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 19; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27, or

(a) a heavy chain variable region (VH) comprising or consisting of an amino acid sequence of SEQ ID NO: 20 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 20; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27, or

(a) a heavy chain variable region (VH) comprising or consisting of an amino acid sequence of SEQ ID NO: 21 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 21; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27, or

(a) a heavy chain variable region (VH) comprising or consisting of an amino acid sequence of SEQ ID NO: 22 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 22; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27, or

(a) a heavy chain variable region (VH) comprising or consisting of an amino acid sequence of SEQ ID NO: 23 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 23; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27, or

(a) a heavy chain variable region (VH) comprising or consisting of an amino acid sequence of SEQ ID NO: 24 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 24; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or

(a) a heavy chain variable region (VH) comprising or consisting of an amino acid sequence of SEQ ID NO: 25 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 25; and (b) a light chain variable region (VL) comprising or consisting of an amino acid sequence of SEQ ID NO: 27, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 27; or

(a) a heavy chain variable region (VH) comprising or consisting of an amino acid sequence of SEQ ID NO: 18 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or

(a) a heavy chain variable region (VH) comprising or consisting of an amino acid sequence of SEQ ID NO: 19 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 19; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or

(a) a heavy chain variable region (VH) comprising or consisting of an amino acid sequence of SEQ ID NO: 20 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 20; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or

(a) a heavy chain variable region (VH) comprising or consisting of an amino acid sequence of SEQ ID NO: 21 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 21; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or

(a) a heavy chain variable region (VH) comprising or consisting of an amino acid sequence of SEQ ID NO: 22 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 22; or (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or

(a) a heavy chain variable region (VH) comprising or consisting of an amino acid sequence of SEQ ID NO: 23 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 23; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or

(a) a heavy chain variable region (VH) comprising or consisting of an amino acid sequence of SEQ ID NO: 24 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 24; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28; or

(a) a heavy chain variable region (VH) comprising or consisting of an amino acid sequence of SEQ ID NO: 25 optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 25; and (b) a light chain variable region (VL) comprising or consisting of an amino acid sequence of SEQ ID NO: 28, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 28.

In a particular aspect, the modifications are substitutions, in particular conservative substitutions.

CH-CL

In one embodiment, the heavy chain (CH) and the light chain (CL) comprises the VL and VH sequences as described hereabove.

In a particular embodiment, the anti-human-PD-1 antibody or antigen binding fragment thereof comprised in the bifunctional molecule comprises:

(a) a heavy chain comprising or consisting of an amino acid sequence selected from the group consisting of SEQ ID NO: 29, 30, 31, 32, 33, 34, 35 or 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, 30, 31, 32, 33, 34, 35 or 36, respectively, and

(b) a light chain comprising or consisting of an amino acid sequence of SEQ ID NO: 37 or SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37 or SEQ ID NO: 38.

In another embodiment, the anti-human-PD-1 humanized antibody or antigen binding fragment thereof comprised in the bifunctional molecule comprises:

(a) a heavy chain comprising or consisting of an amino acid sequence selected from the group consisting of SEQ ID NO: 29, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO:

29, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or

(a) a heavy chain comprising or consisting of an amino acid sequence selected from the group consisting of SEQ ID NO: 30, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 30, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or

(a) a heavy chain comprising or consisting of an amino acid sequence selected from the group consisting of SEQ ID NO: 31, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 31, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or

(a) a heavy chain comprising or consisting of an amino acid sequence selected from the group consisting of SEQ ID NO: 32, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 32, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or

(a) a heavy chain comprising or consisting of an amino acid sequence selected from the group consisting of SEQ ID NO: 33, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 33, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37, or

(a) a heavy chain comprising or consisting of an amino acid sequence selected from the group consisting of SEQ ID NO: 34, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 34, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37, or

(a) a heavy chain comprising or consisting of an amino acid sequence selected from the group consisting of SEQ ID NO: 35, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 35, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37, or

(a) a heavy chain comprising or consisting of an amino acid sequence selected from the group consisting of SEQ ID NO: 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 36, and (b) a light chain comprising or consisting of an amino acid sequence of SEQ ID NO: 37, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37; or

(a) a heavy chain comprising or consisting of an amino acid sequence selected from the group consisting of SEQ ID NO: 29, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38, or

(a) a heavy chain comprising or consisting of an amino acid sequence selected from the group consisting of SEQ ID NO: 30, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 30, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38, or

(a) a heavy chain comprising or consisting of an amino acid sequence selected from the group consisting of SEQ ID NO: 31, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 31, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38, or

(a) a heavy chain comprising or consisting of an amino acid sequence selected from the group consisting of SEQ ID NO: 32, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 32, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38, or

(a) a heavy chain comprising or consisting of an amino acid sequence selected from the group consisting of SEQ ID NO: 33, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO:

33, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38, or

(a) a heavy chain comprising or consisting of an amino acid sequence selected from the group consisting of SEQ ID NO: 34, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 34, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38, or

(a) a heavy chain comprising or consisting of an amino acid sequence selected from the group consisting of SEQ ID NO: 35, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 35, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38, or

(a) a heavy chain comprising or consisting of an amino acid sequence selected from the group consisting of SEQ ID NO: 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 36, and (b) a light chain comprising or consisting of an amino acid sequence of SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 38.

Preferably, the modifications are substitutions, in particular conservative substitutions.

Fc and Hinge Region

Several researches to develop therapeutic antibodies had led to engineer the Fc regions to optimize antibody properties allowing the generation of molecules that are better suited to the pharmacology activity required of them. The Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Several mutations located at the interface between the CH2 and CH3 domains, such as T250Q/M428L and M252Y/S254T/T256E+H433K/N434F, have been shown to increase the binding affinity to FcRn and the half-life of IgG1 in vivo. However, there is not always a direct relationship between increased FcRn binding and improved half-life. One approach to improve the efficacy of a therapeutic antibody is to increase its serum persistence, thereby allowing higher circulating levels, less frequent administration and reduced doses. Engineering Fc regions may be desired to either reduce or increase the effector function of the antibody. For antibodies that target cell-surface molecules, especially those on immune cells, abrogating effector functions is required. Conversely, for antibodies intended for oncology use, increasing effector functions may improve the therapeutic activity. The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions. Binding of IgG to the FcγRs or C1q depends on residues located in the hinge region and the CH2 domain. Two regions of the CH2 domain are critical for FcγRs and C1q binding, and have unique sequences in IgG2 and IgG4.

The antibody according to the invention optionally comprises at least a portion of an immunoglobulin constant region (Fc), typically that of mammalian immunoglobulin, even more preferably a human or humanized immunoglobulin. Preferably, the Fc region is a part of the anti-hPD-1 antibody described herein. The anti-hPD1 antibody or antigen binding fragment thereof comprised in the bifunctional molecule of the invention can include a constant region of an immunoglobulin or a fragment, analog, variant, mutant, or derivative of the constant region. As well known by one skilled in the art, the choice of IgG isotypes of the heavy chain constant domain centers on whether specific functions are required and the need for a suitable in vivo half-life. For example, antibodies designed for selective eradication of cancer cells typically require an active isotype that permits complement activation and effector-mediated cell killing by antibody-dependent cell-mediated cytotoxicity. Both human IgG1 and IgG3 (shorter half-life) isotypes meet these criteria, particularly human IgG1 isotype (wild type and variants). In particular, depending on the IgG isotype of the heavy chain constant domain (particularly human wild type and variants IgG1 isotype), the anti-hPD1 antibody of the invention can be cytotoxic towards cells expressing PD-1 via a CDC, ADCC and/or ADCP mechanism. In fact, the fragment crystallisable (Fc) region interacts with a variety of accessory molecules to mediate indirect effector functions such as antibody-dependent cellular cytotoxicity (ADCC), antibody-dependent cellular phagocytosis (ADCP) and complement-dependent cytotoxicity (CDC).

In preferred embodiments, the constant region is derived from a human immunoglobulin heavy chain, for example, IgG1, IgG2, IgG3, IgG4, or other classes. In a further aspect, the human constant region is selected from the group consisting of IgG1, IgG2, IgG2, IgG3 and IgG4. Preferably, the anti-PD1 antibody comprises an IgG1 or an IgG4 Fc-region. Even more preferably, the anti-hPD1 antibody comprises an IgG4 Fc-region with a S228P that stabilizes the IgG4.

In one embodiment, the anti-PD1 antibody comprises a truncated Fc region or a fragment of the Fc region. In one embodiment, the constant region includes a CH2 domain. In another embodiment, the constant region includes CH2 and CH3 domains or includes hinge-CH2-CH3. Alternatively, the constant region can include all or a portion of the hinge region, the CH2 domain and/or the CH3 domain. In a preferred embodiment, the constant region contains a CH2 and/or a CH3 domain derived from a human IgG4 heavy chain. In some embodiments, the constant region contains a CH2 and/or a CH3 domain derived from a human IgG4 heavy chain.

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes, mutated or not. More preferably the hinge region is derived from a human IgG1 heavy chain. In one embodiment, the constant region includes a CH2 domain derived from a first antibody isotype and a hinge region derived from a second antibody isotype. In a specific embodiment, the CH2 domain is derived from a human IgG2 or IgG4 heavy chain, while the hinge region is derived from an altered human IgG1 heavy chain.

In one embodiment, the constant region contains a mutation that reduces affinity for an Fc receptor or reduces Fc effector function. For example, the constant region can contain a mutation that eliminates the glycosylation site within the constant region of an IgG heavy chain.

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes. The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8). More preferably the hinge region is derived from a human IgG4 heavy chain.

In one embodiment, the constant region includes a CH2 domain derived from a first antibody isotype and a hinge region derived from a second antibody isotype. In a specific embodiment, the CH2 domain is derived from a human IgG4 heavy chain, while the hinge region is derived from an altered human IgG1 heavy chain.

In accordance with the present invention, the constant region can contain CH2 and/or CH3 domains and a hinge region that are derived from different antibody isotypes, i.e., a hybrid constant region. For example, in one embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two cysteines are each mutated to another amino acid. Assembly of such hybrid constant regions has been described in U.S. Patent Publication No. 20030044423, the disclosure of which is hereby incorporated by reference.

In one embodiment, the constant region can contain CH2 and/or CH3 has one of the mutations described in the Table D below, or any combination thereof.

TABLE D
Suitable human engineered Fc domain of an antibody.

| | | | FcR/C1q | Effector |
|---|---|---|---|---|
| Engineered Fc | Isotype | Mutations | Binding | Function |
| hIgG1e1-Fc | IgG1 | T250Q/M428L | Increased | Increased |
| | | | binding to | half-life |
| | | | FcRn | |
| hIgG1e2-Fc | IgG1 | M252Y/S254T/ | Increased | Increased |
| | | T256E + H433K/ | binding to | half-life |
| | | N434F | FcRn | |
| hIgG1e3-Fc | IgG1 | E233P/L234V/ | Reduced | Reduced |
| | | L235A/G236A + | binding to | ADCC and |
| | | A327G/A330S/ | FcγRI | CDC |
| | | P331S | | |
| hIgG1e4-Fc | IgG1 | E333A | Increased | Increased |
| | | | binding to | ADCC and |
| | | | FcγRIIIa | CDC |
| hIgG1e5-Fc | IgG1 | S239D/A330L/ | Increased | Increased |
| | | I332E | binding to | ADCC |
| | | | FcγRIIIa | |
| hIgG1e6-Fc | IgG1 | P257I/Q311 | Increased | Unchanged |
| | | | binding to | half-life |
| | | | FcRn | |
| hIgG1e7-Fc | IgG1 | K326W/E333S | Increased | Increased |
| | | | binding to | CDC |
| | | | C1q | |
| hIgG1e9-Fc | IgG1 | S239D/I332E/ | Increased | Increased |
| | | G236A | FcγRIIa/ | macrophage |
| | | | FcγRIIb | phagocytosis |
| | | | ratio | |
| hIgG1e9-Fc | IgG1 | N297A | Reduced | Reduced |
| | | | binding to | ADCC and |
| | | | FcγRI | CDC |
| hIgG1e9-Fc | IgG1 | LALA | Reduced | Reduced |
| | | (L234A/L235A) | binding to | ADCC and |
| | | | FcγRI | CDC |
| hIgG1e10-Fc | IgG1 | N297A + YTE | Reduced | Reduced |
| | | (N298A + | binding to | ADCC and |
| | | M252Y/S254T/ | FcγRI | CDC |
| | | T256E) | Increased | Increased |
| | | | binding to | half-life |
| | | | FcRn | |
| hIgG1e11-Fc | IgG1 | K322A | Reduced | Reduced |
| | | | binding to | CDC |
| | | | C1q | |
| hIgG2e1-Fc | IgG4 | S228P | — | Reduced |
| | | | | Fab-arm |
| | | | | exchange |
| hIgG4e1-Fc | IgG4 | LALA | Increased | Increased |
| | | (L234A/L235A) | binding to | half-life |
| | | | FcRn | |
| hIgG4e2-Fc | IgG4 | S228P + YTE | Increased | Reduced |
| | | (S228P + | binding to | Fab-arm |
| | | M252Y/S254T/ | FcRn | exchange |
| | | T256E) | | Increased |
| | | | | half-life |
| hIgG4e3-Fc | IgG4 | K444A | | Abolish |
| | | | | cleavage |
| | | | | of the |
| | | | | C-terminal |
| | | | | lysine of the |
| | | | | antibody |
| hIgG1e112-Fc | IgG4 | K444A | | Abolish |
| | | | | cleavage |
| | | | | of the |
| | | | | C-terminal |
| | | | | lysine of the |
| | | | | antibody |

Numbering of residues in the heavy chain constant region is according to EU numbering (Edelman, G. M. et al., Proc. Natl. Acad. USA, 63, 78-85 (1969); see Worldwide Website: imgt.org/IMGTScientificChart/Numbering/ Hu_IGHGnber.html#refs).

In a particular aspect, the bifunctional molecule, preferably the binding moiety, comprises a human IgG1 heavy chain constant domain or an IgG1 Fc domain, optionally with a substitution or a combination of substitutions selected from the group consisting of T250Q/M428L; M252Y/S254T/T256E +H433K/N434F; E233P/L234V/L235A/G236A+A327G/A330S/P331S; E333A; S239D/A330L/I332E; P257I/Q311; K326W/E333S; S239D/1332E/G236A; N297A; L234A/L235A; N297A +M252Y/S254T/T256E; K322A and K444A, preferably selected from the group consisting of N297A optionally in combination with M252Y/S254T/T256E, and L234A/L235A.

In another aspect, the binding moiety comprises a human IgG4 heavy chain constant domain or a human IgG4 Fc domain, optionally with a substitution or a combination of substitutions selected from the group consisting of S228P; L234A/L235A, S228P +M252Y/S254T/T256E and K444A. Even more preferably, the bifunctional molecule, preferably the binding moiety, comprises an IgG4 Fc-region with a S228P that stabilizes the IgG4.

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In one embodiment, the constant region contains a mutation that reduces affinity for an Fc receptor or reduces Fc effector function. For example, the constant region can contain a mutation that eliminates the glycosylation site within the constant region of an IgG heavy chain. Preferably, the CH2 domain contains a mutation that eliminates the glycosylation site within the CH2 domain.

In one embodiment, the anti-hPD1 according to the invention has a heavy chain constant domain of SEQ ID NO: 39 or 52 and/or a light chain constant domain of SEQ ID NO: 40, particularly a heavy chain constant domain of SEQ ID NO: 39 or 52 and a light chain constant domain of SEQ ID NO: 40.

In another embodiment, the anti-hPD1 according to the invention has a heavy chain constant domain of SEQ ID NO: 52 and/or a light chain constant domain of SEQ ID NO: 40, particularly a heavy chain constant domain of SEQ ID NO: 52 and a light chain constant domain of SEQ ID NO: 40.

TABLE E
Example of a heavy chain constant domain and a light chain constant
domain suitable for the humanized antibodies according to the invention.

| | |
|---|---|
| Heavy chain constant | ASTKGPSVFPLAPCSRSTSESTAALGCLVKDYFPEPVTVSWNSGALTSGVHTFPAVL |
| domain (IgG4m-S228P) | QSSGLYSLSSVVTVPSSSLGTKTYTCNVDHKPSNTKVDKRVESKYGPPCPPCPAPEFL |
| SEQ ID NO: 39 | GGPSVFLFPPKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVLDSDGS |
| | FFLYSRLTVDKSRWQEGNVFSCSVMHEALHNHYTQKSLSLSPGK |
| Light chain constant | RTVAAPSVFIFPPSDEQLKSGTASVVCLLNNFYPREAKVQWKVDNALQSGNSQESV |
| domain (CLkappa) | TEQDSKDSTYSLSSTLTLSKADYEKHKVYACEVTHQGLSSPVTKSFNRGEC |
| SEQ ID NO: 40 | |
| Heavy chain constant | ASTKGPSVFPLAPSSKSTSGGTAALGCLVKDYFPEPVTVSWNSGALTSGVHTFPAVL |
| domain (IgG1m- | QSSGLYSLSSVVTVPSSSLGTQTYICNVNHKPSNTKVDKKVEPKSCDKTHTCPPCPAP |
| N298A) | ELLGGPSVFLFPPKPKDTLMISRTPEVTCVVVDVSHEDPEVKFNWYVDGVEVHNAK |
| SEQ ID NO: 52 | TKPREEQYASTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |

The alteration of amino acids near the junction of the Fc portion and the non-Fc portion can dramatically increase the serum half-life of the Fc fusion protein (PCT publication WO 01/58957). Accordingly, the junction region of a protein or polypeptide of the present invention can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life.

All subclass of Human IgG carries a C-terminal lysine residue of the antibody heavy chain (K444) that are cleaved off in circulation. This cleavage in the blood may compromise the bioactivity of the bifunctional molecule by releasing IL-7. To circumvent this issue, K444 amino acid in the IgG1 or IgG4 domain may be substituted by an alanine to reduce proteolytic cleavage, a mutation commonly used for antibodies. Then, in one embodiment, the anti-PD1 antibody comprises at least one further amino acid substitution consisting of K444A.

In one embodiment, the anti-PD1 antibody comprises an additional cysteine residue at the C-terminal domain of the IgG to create an additional disulfide bond and potentially restrict the flexibility of the bifunctional molecule.

In certain embodiments, an antibody may be altered to increase, decrease or eliminate the extent to which it is glycosylated.

Checkpoint Inhibitor

The inventors show herein that the bifunctional molecule according to the invention combines the effect of the IL-7 variant or mutant on the IL-7 receptor and the blockade of the inhibitory effect of PD-1, and is suitable for optimizing the effect of a checkpoint inhibitor such as anti-PD-1 antibody. In particular, a synergistic effect on the activation of T cells, especially exhausted T cells, more particularly on the TCR signalling has been shown. The inventors particularly show an activation on the same cell, provided by the binding of the anti-PD-1 antibody and of IL-7 comprised in the bifunctional molecule on the same immune cell. This synergistic effect has never been observed using IL-7 and anti-PD-1 antibodies as separate compounds. Then, it can be envisioned that any molecule other than PD-1 that is expressed on immune cell expressing IL-7R may be triggered by a bifunctional construct according to the invention, particularly a factor of exhaustion. Then, in embodiment, the bifunctional molecule comprises an antibody or antigen binding fragment thereof that is directed against a target expressed on immune cells, other than PD-1. For example, the target can be a receptor expressed at the surface of the immune cells, especially T cells. The receptor can be an inhibitor receptor. Alternatively, the receptor can be an activating receptor.

As used herein, the term “target” refers to a peptide, polypeptide, protein, antigen or epitope that is expressed on the external surface of immune cells. With regards to the expression of a target on the surface of immune cells, the term “expressed” refers to a target present or presented at the outer surface of a cell. The term “specifically expressed” mean that the target is expressed on immune cells, but is not substantially expressed by other cell type, particularly such as tumoral cells.

In one embodiment, the target is specifically expressed by immune cells in a healthy subject or in a subject suffering from a disease, in particular such as a cancer. This means that the target has a higher expression level in immune cells than in other cells or that the ratio of immune cells expressing the target by the total immune cells is higher than the ratio of other cells expressing the target by the total other cells. Preferably the expression level or ratio is higher by a factor 2, 5, 10, 20, 50 or 100. More specifically, it can be determined for a particular type of immune cells, for instance T cells, more specifically CD8+ T cells, effector T cells or exhausted T cells, or in a particular context, for instance a subject suffering of a disease such as a cancer or an infection.

In one aspect, the target is an immune checkpoint. Preferably, the target is selected from the group consisting of PD-1, CD28, CD80, CTLA-4, BTLA, TIGIT, CD160, CD40L, ICOS, CD27, OX40, 4-16B, GITR, HVEM, Tim-1, LFA-1, TIM3, CD39, CD30, NKG2D, LAG3, B7-1, 2B4, DR3, CD101, CD44, SIRPG, CD28H, CD38, CXCRS, CD3, PDL2, CD4 and CD8. Such targets are more particularly described in the Table F below.

TABLE F
Example of target of interest.

| | | Uniprot |
|---|---|---|
| Name | Official name | reference |
| 2B4 | Natural killer cell receptor 2B4 (NK cell type I | Q07763 |
| | receptor protein 2B4, NKR2B4) (Non-MHC | |
| | restricted killing associated) (SLAM family | |
| | member 4, SLAMF4) (Signaling lymphocytic | |
| | activation molecule 4) (CD antigen CD244) | |
| 4-1BB | Tumor necrosis factor receptor superfamily | Q07011 |
| | member 9 (4-1BB ligand receptor, CD137) | |
| BTLA | B- and T-lymphocyte attenuator (B- and T- | Q7Z6A9 |
| | lymphocyte-associated protein) (CD antigen | |
| | CD272) | |
| CD101 | Immunoglobulin superfamily member 2, IgSF2 | Q93033 |
| | (Cell surface glycoprotein V7) (Glu-Trp-Ile EWI | |
| | motif-containing protein 101, EWI-101) (CD | |
| | antigen CD101) | |
| CD160 | CD160 antigen (Natural killer cell receptor BY55) | O95971 |
| CD27 | CD27 antigen (CD27L receptor) (T-cell activation | P26842 |
| | antigen CD27) (T14) (Tumor necrosis factor | |
| | receptor superfamily member 7) (CD antigen | |
| | CD27) | |
| CD28 | T-cell-specific surface glycoprotein CD28 (TP44) | P10747 |
| CD28H | Transmembrane and immunoglobulin domain- | Q96BF3 |
| | containing protein 2 (CD28 homolog) | |
| | (Immunoglobulin and proline-rich receptor 1, | |
| | IGPR-1) | |
| CD3 | T-cell surface glycoprotein CD3 | P07766 |
| | | (CD3e) |
| | | P04234 |
| | | (CD3d) |
| | | P09693 |
| | | (CD3g) |
| CD30 | Tumor necrosis factor ligand superfamily member | P32971 |
| | 8 (CD30 ligand, CD30-L) (CD antigen CD153) | |
| CD38 | ADP-ribosyl cyclase/cyclic ADP-ribose hydrolase | P28907 |
| | 1 (ADPRC 1, cADPr hydrolase 1) | |
| CD39 | Ectonucleoside triphosphate diphosphohydrolase-1 | P49961 |
| | (NTPDase 1, Ecto-apyrase, ATPDase 1, or | |
| | Lymphoid cell activation antigen) | |
| CD4 | T-cell surface glycoprotein CD4 (T-cell surface | P01730 |
| | antigen T4/Leu-3) | |
| CD40L | CD40 ligand (T-cell antigen Gp39, TNF-related | P29965 |
| | activation protein, Tumor necrosis factor ligand | |
| | superfamily member 5, CD154) | |
| CD44 | CD44 antigen (Epican, Extracellular matrix | P16070 |
| | receptor III, GP90 lymphocyte homing/adhesion | |
| | receptor, HUTCH-1, Heparan sulfate | |
| | proteoglycan, Hermes antigen, Hyaluronate | |
| | receptor, Phagocytic glycoprotein 1, Phagocytic | |
| | glycoprotein 1) | |
| CD8 | T-cell surface glycoprotein CD8 | P01732 |
| | | (CD8a) |
| | | P10966 |
| | | (CD8b) |
| CD80 | T-lymphocyte activation antigen CD80 | P33681 |
| | (Activation B7-1 antigen, BB1, CTLA-4 counter- | |
| | receptor B7.1, B7) | |
| CTLA-4 | Cytotoxic T-lymphocyte protein 4 (Cytotoxic T- | P16410 |
| | lymphocyte-associated antigen 4, CTLA-4) (CD | |
| | antigen CD152) | |
| CXCR5 | C-X-C chemokine receptor type 5 (Burkitt | P32302 |
| | lymphoma receptor 1, Monocyte-derived receptor | |
| | 15, CD185) | |
| DR3 | Death receptor 3 (Tumor necrosis factor receptor | Q93038 |
| | superfamily member 25, WSL, Apo-3, LARD) | |
| GITR | Tumor necrosis factor receptor superfamily | Q9Y5U5 |
| | member 18 (Activation-inducible TNFR family | |
| | receptor, Glucocorticoid-induced TNFR-related | |
| | protein, CD357) | |
| HVEM | Tumor necrosis factor receptor superfamily | Q92956 |
| | member 14 (Herpes virus entry mediator A, | |
| | Herpesvirus entry mediator A, HveA) (Tumor | |
| | necrosis factor receptor-like 2, TR2) (CD antigen | |
| | CD270) | |
| ICOS | Inducible T-cell costimulator (Activation- | Q9Y6W8 |
| | inducible lymphocyte immunomediatory | |
| | molecule, CD278) | |
| LAG3 | Lymphocyte activation gene 3 protein, LAG-3 | P18627 |
| | (Protein FDC) (CD antigen CD223) | |
| LFA-1 | Leukocyte adhesion glycoprotein LFA-1 alpha | P20701 |
| | chain (Integrin alpha-L, CD11 antigen-like family | |
| | member A) | |
| NKG2D | NKG2-D type II integral membrane protein (Killer | P26718 |
| | cell lectin-like receptor subfamily K member 1, | |
| | NK cell receptor D, NKG2-D-activating NK | |
| | receptor, CD314) | |
| OX40 | Tumor necrosis factor receptor superfamily | P43489 |
| | member 4 (ACT35 antigen, AX transcriptionally- | |
| | activated glycoprotein 1 receptor) | |
| PD-1 | Programmed cell death protein 1 (CD279) | Q15116 |
| PDL2 | Programmed cell death 1 ligand 2, PD-1 ligand 2, | Q9BQ51 |
| | PD-L2, PDCD1 ligand 2, Programmed death | |
| | ligand 2 (Butyrophilin B7-DC, B7-DC) (CD | |
| | antigen CD273) | |
| SIRPG | Signal-regulatory protein gamma, SIRP-gamma | Q9P1W8 |
| | (CD172 antigen-like family member B) (Signal- | |
| | regulatory protein beta-2, SIRP-b2, SIRP-beta-2) | |
| | (CD antigen CD172g) | |
| TIGIT | T-cell immunoreceptor with 1 g and ITIM | Q495A1 |
| | domains (V-set and immunoglobulin domain- | |
| | containing protein 9) (V-set and transmembrane | |
| | domain-containing protein 3) | |
| Tim-1 | Hepatitis A virus cellular receptor 1 (T-cell | Q96D42 |
| | immunoglobulin and mucin domain-containing | |
| | protein 1, Kidney injury molecule 1, KIM-1, T- | |
| | cell immunoglobulin mucin receptor 1, T-cell | |
| | membrane protein 1, CD365) | |
| TIM3 | Hepatitis A virus cellular receptor 2, HAVcr-2 (T- | Q8TDQ0 |
| | cell immunoglobulin and mucin domain- | |
| | containing protein 3, TIMD-3) (T-cell | |
| | immunoglobulin mucin receptor 3, TIM-3) (T-cell | |
| | membrane protein 3) | |

Then, in this aspect, the antibody or the antigen fragment thereof comprised in the bifunctional molecule according to the invention binds a target selected from the group consisting CD28, CD80, CTLA-4, BTLA, TIGIT, CD160, CD4OL, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8.

In a preferred aspect, the antibody or antigen binding fragment thereof comprised in the bifunctional molecule according to the invention is selected from the group consisting of CTLA-4, BTLA, TIGIT, LAG3 and TIM3.

Antibodies directed against TIM3 and bifunctional or bispecific molecules targeting TIM3 are also known such as Sym023, TSR-022, MBG453, LY3321367, INCAGN02390, BGTB-A425, LY3321367, RG7769 (Roche).

In some embodiments, a TFM-3 antibody is as disclosed in International Patent Application Publication Nos. WO2013006490, WO2016/161270, WO 2018/085469, or WO 2018/129553, WO 2011/155607, U.S. Pat. No. 8,552,156, EP 2581113 and U.S. 2014/044728.

Antibodies directed against CTLA-4 and bifunctional or bispecific molecules targeting CTLA-4 are also known such as ipilimumab, tremelimumab, MK-1308, AGEN-1884, XmAb20717 (Xencor), MED15752 (AstraZeneca). Anti-CTLA-4 antibodies are also disclosed in WO18025178, WO19179388, WO19179391, WO19174603, WO19148444, WO19120232, WO19056281, WO19023482, WO18209701, WO18165895, WO18160536, WO18156250, WO18106862, WO18106864, WO18068182, WO18035710, WO18025178, WO17194265, WO17106372, WO17084078, WO17087588, WO16196237, WO16130898, WO16015675, WO12120125, WO09100140 and WO07008463.

Antibodies directed against LAG-3 and bifunctional or bispecific molecules targeting LAG-3 are also known such as BMS- 986016, IMP701, MGD012 or MGD013 (bispecific PD-1 and LAG-3 antibody). Anti-LAG-3 antibodies are also disclosed in WO2008132601, EP2320940, WO19152574.

Antibodies directed against BTLA are also known in the art such as hu Mab8D5, hu Mab8A3, hu Mab21H6, hu Mab19A7, or hu Mab4C7. The antibody TABOO4 against BTLA are currently under clinical trial in subjects with advanced malignancies. Anti-BTLA antibodies are also disclosed in WO08076560, WO10106051 (e.g., BTLA8.2), WO11014438 (e.g., 4C7), WO17096017 and WO17144668 (e.g., 629.3). Antibodies directed against TIGIT are also known in the art, such as BMS-986207 or AB154, BMS-986207 CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8 as disclosed in WO19232484.

Anti-TIGIT antibodies are also disclosed in WO16028656, WO16106302, WO16191643, WO17030823, WO17037707, WO17053748, WO17152088, WO18033798, WO18102536, WO18102746, WO18160704, WO18200430, WO18204363, WO19023504, WO19062832, WO19129221, WO19129261, WO19137548, WO19152574, WO19154415, WO19168382 and WO19215728.

Antibodies directed against CD160 are also known in the art, such as CL1-R2 CNCM 1-3204 as disclosed in WO06015886, or others as disclosed in WO10006071, WO10084158, WO18077926.

In a particular aspect, the bifunctional molecule according to the invention comprises an anti-CTLA-4 antibody or antigen binding fragment thereof, preferably a human, humanized or chimeric anti-CTLA-4 antibody or antigen binding fragment thereof. Preferably, the antibody is an antagonist of CTLA-4. Therefore, the bifunctional molecule combines the effect of the IL-7wt, variant or mutant thereof, on the IL-7 receptor and the blockade of the inhibitory effect of CTLA-4, and may have a synergistic effect on the activation of T cells, especially exhausted T cells, more particularly on the TCR signaling.

In another particular aspect, the bifunctional molecule according to the invention comprises an anti-BTLA antibody or antigen binding fragment thereof, preferably a human, humanized or chimeric anti-BTLA antibody or antigen binding fragment thereof. Preferably, the antibody is an antagonist of BTLA. Therefore, the bifunctional molecule combines the effect of the IL-7wt, variant or mutant thereof on the IL-7 receptor and the blockade of the inhibitory effect of BTLA, and may have a synergistic effect on the activation of T cells, especially exhausted T cells, more particularly on the TCR signaling.

In another particular aspect, the bifunctional molecule according to the invention comprises an anti-TIGIT antibody or antigen binding fragment thereof, preferably a human, humanized or chimeric anti-TIGIT antibody or antigen binding fragment thereof. Preferably, the antibody is an antagonist of TIGIT. Therefore, the bifunctional molecule combines the effect of the IL-7wt, variant or mutant thereof, on the IL-7 receptor and the blockade of the inhibitory effect of TIGIT, and may have a synergistic effect on the activation of T cells, especially exhausted T cells, more particularly on the TCR signaling.

In another particular aspect, the bifunctional molecule according to the invention comprises an anti-LAG-3 antibody or antigen binding fragment thereof, preferably a human, humanized or chimeric anti-LAG-3 antibody or antigen binding fragment thereof. Preferably, the antibody is an antagonist of LAG-3. Therefore, the bifunctional molecule combines the effect of the IL-7wt, variant or mutant thereof, on the IL-7 receptor and the blockade of the inhibitory effect of LAG-3, and may have a synergistic effect on the activation of T cells, especially exhausted T cells, more particularly on the TCR signaling.

In another particular aspect, the bifunctional molecule according to the invention comprises an anti-TIM3 antibody or antigen binding fragment thereof, preferably a human, humanized or chimeric anti-TIM3 antibody or antigen binding fragment thereof. Preferably, the antibody is an antagonist of TIM3. Therefore, the bifunctional molecule combines the effect of the IL-7 variant or mutant on the IL-7 receptor and the blockade of the inhibitory effect of TIM3, and may have a synergistic effect on the activation of T cells, especially exhausted T cells, more particularly on the TCR signaling.

Peptide Linker

This invention includes a bifunctional molecule which may comprise a peptide linker between the anti-PD-1 antibody or fragment thereof and IL-7. The peptide linker usually has a length and flexibility enough to ensure that the two protein elements connected with the linker in between have enough freedom in space to exert their functions and avoid influences of the formation of a-helix and β-fold on the stability of the recombinant bifunctional molecule.

In an aspect of the disclosure, the anti-hPD1 antibody is preferably linked to IL-7 by a peptide linker. In other words, the invention relates to bifunctional molecule comprising an anti-PD1 antibody as detailed herein or an antigen binding fragment thereof, with a chain, e.g., the light or heavy chain or a fragment thereof, preferably the heavy chain or a fragment thereof, is linked to IL-7 through a peptide linker. As used herein, the term “linker” refers to a sequence of at least one amino acid that links IL-7 and the anti-PD-1 immunoglobulin sequence portion. Such a linker may be useful to prevent steric hindrances. The linker is usually 3-44 amino acid residues in length. Preferably, the linker has 3-30 amino acid residues. In some embodiments, the linker has 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

In an embodiment, the invention relates to a bifunctional molecule comprising an anti-PD-1 antibody or antigen-binding fragment thereof as defined above and IL-7, wherein a chain of the antibody, e.g., the light or heavy chain, preferably the heavy chain, even more preferably the C-terminus of the heavy or light chain is linked to IL-7, preferably to the N-terminus of IL-7, by a peptide linker.

In a particular aspect, the invention relates to a bifunctional molecule comprising an anti-hPD-1 antibody or antigen-binding fragment thereof as defined above, wherein IL-7 is linked to the C-terminal end of the heavy chain of said antibody (e.g., the C-terminal end of the heavy chain constant domain), preferably by a peptide linker.

In an embodiment, the invention relates to bifunctional molecule comprising an anti-PD-1 antibody or antigen-binding fragment thereof as defined above, wherein IL-7 is linked to the C-terminal end of the light chain of said antibody (e.g., the C-terminal end of the light chain constant domain), preferably by a peptide linker.

The linker sequence may be a naturally occurring sequence or a non-naturally occurring sequence. If used for therapeutic purposes, the linker is preferably non-immunogenic in the subject to which the bifunctional molecule is administered. One useful group of linker sequences are linkers derived from the hinge region of heavy chain antibodies as described in WO 96/34103 and WO 94/04678. Other examples are poly-alanine linker sequences. Further preferred examples of linker sequences are Gly/Ser linkers of different length including (Gly4Ser)₄, (Gly4Ser)₃, (Gly4Ser)₂, Gly4Ser, Gly3Ser, Gly3, Gly2ser and (Gly3Ser2)₃, in particular (Gly4Ser)₃. Preferably, the linker is selected from the group consisting of (Gly4Ser)₄, (Gly4Ser)₃, and (Gly3Ser2)₃.

In one embodiment, the linker comprised in the bifunctional molecule is selected in the group consisting of (Gly4Ser)₄, (Gly4Ser)₃, (Gly4Ser)₂, Gly4Ser, Gly3Ser, Gly3, Gly2ser and (Gly3Ser2)₃, preferably is (Gly4Ser)₃. Preferably, the linker is selected from the group consisting of (Gly4Ser)₄, (Gly4Ser)₃, and (Gly3Ser2)₃. Even more preferably, the linker is (GGGGS)3.

In an embodiment, the invention relates to a bifunctional molecule that comprises an anti-PD-1 antibody or a fragment thereof as defined above wherein the antibody or a fragment thereof is linked to IL-7 by a linker sequence, preferably selected from the group consisting of (GGGGS)₃, (GGGGS)₄, (GGGGS)₂, GGGGS, GGGS, GGG, GGS and (GGGS)₃, even more preferably by (GGGGS)₃. Preferably, the linker is selected from the group consisting of (GGGGS)₃, (GGGGS)₄, and (GGGS)₃.

Preferably, the heavy chain, preferably the C terminus of the heavy chain of the anti-PD-1 antibody is genetically fused via a flexible (Gly₄Ser)₃ linker to the N-terminus of IL-7. At the fusion junction, the C-terminal lysine residue of the antibody heavy chain can be mutated to alanine to reduce proteolytic cleavage.

Preferably, the heavy chain, preferably the C terminus of the light chain of the anti-PD-1 antibody is genetically fused via a flexible (Gly₄Ser)₃ linker to the N-terminus of IL-7. At the fusion junction, the C-terminal lysine residue of the antibody light chain can be mutated to alanine to reduce proteolytic cleavage.

IL-7

The bifunctional molecule according to the invention comprises an additional or second entity that comprises an interleukin 7, or a variant or fragment thereof.

Preferably, the IL-7 protein is a human IL-7 or variants thereof. Accordingly, the IL-7 or variant thereof has an amino acid sequence having at least 75% of identity with the wild type IL-7, especially with the protein of SEQ ID NO: 51.

In one embodiment, the bifunctional molecule comprises the typical wild-type IL-7 human protein of 152 amino acids (SEQ ID NO: 51). Preferably the IL-7 protein is the protein of SEQ ID NO: 51. The IL-7 proteins can comprise its peptide signal or be devoid of it.

A “variant” of an IL-7 protein is defined as an amino acid sequence that is altered by one or more amino acids. The variant can have “conservative” modifications or “non-conservative” modifications. Such modifications can include amino acid substitution, deletions and/or insertions. Guidance in determining which and how many amino acid residues may be substituted, inserted or deleted without abolishing biological properties (e.g. activity, binding capacity and/or structure) can be found using computer programs well known in the art, for example software for molecular modeling or for producing alignments. In a particular aspect, the variant IL-7 proteins included within the invention specifically include IL-7 proteins that retain substantially equivalent biological IL-7 property in comparison to a wild-type IL-7. In an alternative aspect, the variant IL-7 proteins included within the invention specifically include IL-7 proteins that do not retain substantially equivalent biological property (e.g. activity, binding capacity and/or structure) in comparison to a wild-type IL-7. A variant of IL-7 also include altered polypeptides sequence of IL-7 (e.g. oxidized, reduced, deaminated or truncated forms). Particularly, truncations or fragment of IL-7 which retain comparable biological property as the full-length IL-7 protein are included within the scope of the invention. In one embodiment, the interleukin 7 is any biological active fragment thereof. Variants of IL-7 include, more preferably, natural allelic variants resulting from natural polymorphism, including SNPs, splicing variants, etc.

The biological activity of IL-7 protein can be measured using in vitro cellular proliferation assays. Preferably, the IL-7 variants according to the invention maintain biological activity of at least 1%, 5%, 10% , 20%, 30%, 40%, 50%, 60% in comparison with the wild type human IL-7, preferably at least 80%, 90%, 95% and even more preferably 99% in comparison with the wild type IL-7.

Variant IL-7 proteins also include polypeptides that have at least about 65%, 70%, 75%, 80%, 85%, 90%, 92%, 95%, 96%, 97%, 98%, 99%, or more sequence identity with wild-type IL-7, especially with the protein of SEQ ID NO: 51.

Preferred IL-7 according to the invention are human IL-7 polypeptides comprising or consisting of an amino acid sequence as described in SEQ ID NO: 51, EP 314 415 or in WO2004/018681 A2, as well as any natural variants and homologs thereof.

In one aspect, the IL-7 polypeptide used in the present invention is a recombinant IL-7. The term “recombinant”, as used herein, means that the polypeptide is obtained or derived from a recombinant expression system, i.e., from a culture of host cells (e.g., microbial or insect or plant or mammalian) or from transgenic plants or animals engineered to contain a nucleic acid molecule encoding an IL-7 polypeptide. Preferably, the recombinant IL-7 is a human recombinant IL-7, (e.g. a human IL-7 produced in recombinant expression system).

The invention also provides bifunctional molecules that comprises IL-7 proteins that have an enhanced biological activity compared to wild-type IL-7 proteins. For example, as described in U.S. Pat. No. 7,960,514, IL-7 proteins having the disulfide bonding pattern of Cys2-Cys92, Cys34-Cys129 and Cys47-141 are more active in vivo than a wild-type recombinant IL-7 protein. Hyperglycosylation of IL-7 such as described in EP1904635 also improve IL-7 biological activity such as IL-7 proteins where Asn116 is non-glycosylated but Asn70 and Asn91 are glycosylated.

Alternatively, the invention provides bifunctional molecules that comprises IL-7 proteins that have a reduced immunogenicity compared to wild-type IL-7 proteins, particularly by the removing T-cell epitopes within IL-7 that may stimulate to an immune response. Examples of such IL-7 are described in WO 2006061219.

In a particular aspect, the present disclosure also provides a bifunctional molecule comprising an IL-7 variant or mutant. The terms “interleukin-7 mutant”, “mutated IL-7”, “IL-7 mutant”, “IL-7 variant”, “IL-7m” or “IL-7v” are used interchangeably herein.

In this context, the IL-7 variant or mutant does not retain substantially equivalent biological property (e.g. activity, binding capacity and/or structure) in comparison to a wild-type IL-7. The IL-7 mutant or variant comprises at least one mutation. Particularly, the at least one mutation decreases the affinity of IL-7 variant or mutant to IL-7 receptor (IL-7R) but does not lead to the loss of the recognition of IL-7R. Accordingly, the IL-7 mutant or variant retains a capacity to activate IL-7R, for instance as measured by the pStat5 signal, for example such as disclosed in Bitar et al., Front. Immunol., 2019, volume 10). The biological activity of IL-7 protein can be measured using in vitro cellular proliferation assays or by measuring the P-Stat5 into the T cells by ELISA or FACS. Preferably, the IL-7 variants according to the invention has reduced biological properties (e.g. activity, binding capacity and/or structure) by at least a factor 2, 5, 10, 20, 30, 40, 50, 100, 250, 500, 750,1000, 2500, 5000, or 8000 in comparison with the wild type IL-7, preferably the wth-IL7. More preferably, the IL-7 variants have a reduced binding to the IL-7 receptor but retains a capacity to activate IL-7R. For instance, the binding to the IL-7 receptor can be reduced by at least 1%, 5%, 10%, 20%, 30%, 40%, 50%, 60% in comparison with the wild type IL-7, and retains a capacity to activate IL-7R by at least 90%, 80%, 70%, 60%, 50%, 40%, 30% or 20% in comparison with the wild type IL-7.

In one aspect, the IL-7 variant or mutant differs from wt-IL-7 by at least one amino acid mutation which i) reduces affinity of the IL-7 variant for IL-7 receptor (IL-7R) in comparison to the affinity of wt-IL-7 for IL-7R, and ii) improves pharmacokinetics of the IL7 variant in comparison to the wt-IL7. More particularly, the IL-7 variant or mutant further retains the capacity to activate IL-7R, in particular through the pStat5 signaling.

In another aspect, the bifunctional molecule comprising an IL-7 variant or mutant differs from a wt-IL-7 by at least one amino acid mutation which i) reduces affinity of the bifunctional molecule for IL-7 receptor (IL-7R) in comparison to the affinity for IL-7R of a bifunctional molecule comprising wt-IL-7, and ii) improves pharmacokinetics of the bifunctional molecule comprising an IL-7 variant or mutant in comparison to the bifunctional molecule comprising wt-IL-7. More particularly, the bifunctional molecule comprising an IL-7 variant or mutant further retains the capacity to activate IL-7R, in particular through the pStat5 signaling. For instance, the binding bifunctional molecule comprising an IL-7 variant or mutant to the IL-7 receptor can be reduced by at least 10%, 20%, 30%, 40%, 50%, 60% in comparison with the bifunctional molecule comprising a wild type IL-7, and retains a capacity to activate IL-7R by at least 90%, 80%, 70%, 60%, 50%, 40%, 30% or 20% in comparison with the bifunctional molecule comprising a wild type IL-7.

In a particular aspect, the IL-7 variant or mutant presents a reduced affinity for IL-7 receptor (IL-7R) in comparison to the affinity of wth-IL-7 for IL-7R. In particular, the IL-7 variant or mutant presents a reduced affinity for CD127 and/or CD132 in comparison to the affinity of wth-IL-7 for CD127 and/or CD132, respectively. Preferably, the IL-7 variant or mutant presents a reduced affinity for CD127 in comparison to the affinity of wth-IL-7 for CD127.

Preferably, the at least one amino acid mutation decreases the affinity of IL-7 variant or mutant for IL-7R, in particular CD132 or CD127, by at least a factor 10, 100, 1000, 10 000 or 100 000 in comparison to the affinity of wt-IL-7 for IL-7R. Such affinity comparison may be performed by any methods known by the skilled of the art, such as ELISA or Biacore.

Preferably, the at least one amino acid mutation decreases affinity of IL-7 variant or mutant for IL-7R but do not decrease the biological activity of IL-7 variant or mutant in comparison to IL-7 wt, in particular as measured by pStat5 signal.

Alternatively, the at least one amino acid mutation decreases affinity of IL-7 variant or mutant for IL-7R but do not decrease significatively the biological activity of IL-7m in comparison to IL-7 wt, in particular as measured by pStat5 signal.

Additionally or alternatively, the IL-7 variant or mutant improves pharmacokinetics of the bifunctional molecule comprising the IL-7 variant or mutant in comparison with a bifunctional molecule comprising a wild type IL-7. Particularly, the IL-7 variant or mutant according to the invention improves pharmacokinetics of the bifunctional molecule comprising IL-7 variant or mutant by at least a factor 10, 100 or 1000 in comparison with a bifunctional molecule comprising wth-IL-7. Pharmacokinetics profile comparison may be performed by any methods known by the skilled of the art, such as in vivo injection of the drug and dosage ELISA of the drug in the sera at multiple time point, for example as shown in example 9.

As used herein, the terms “pharmacokinetics” and “PK” are used interchangeably and refer to the fate of compounds, substances or drugs administered to a living organism. Pharmacokinetics particularly comprise the ADME or LADME scheme, which stands for Liberation (i.e. the release of a substance from a composition), Absorption (i.e. the entrance of the substance in blood circulation), Distribution (i.e. dispersion or dissemination of the substance trough the body) Metabolism (i.e. transformation or degradation of the substance) and Excretion (i.e. the removal or clearance of the substance from the organism). The two phases of metabolism and excretion can also be grouped together under the title elimination. Different pharmacokinetics parameters can be monitored by the man skilled in the art, such as elimination half-life, elimination constant rate, clearance (i.e. the volume of plasma cleared of the drug per unit time), Cmax (Maximum serum concentration), and Drug exposure (determined by Area under the curve) (Scheff et al, Pharm Res., 2011, 28, 1081-9).

Then, the improvement of the pharmacokinetics by the use of IL-7 variant or mutant refers to the improvement of at least one of the above-mentioned parameters. Preferably, it refers to the improvement of the elimination half-life of the bifunctional molecule, i.e. the increase of half-life duration, or of Cmax.

In a particular embodiment, the at least one mutation of IL-7 variant or mutant improves the elimination half-life of a bifunctional molecule comprising IL-7 variant or mutant in comparison to a bifunctional molecule comprising IL-7 wt.

In one embodiment, the IL-7 variant or mutant presents at least 75%, at least 80%, at least 85%, at least 90%, at least 95%, at least 97%, at least 98% or at least 99% of identity with the wild-type human IL-7 (wth-IL-7) protein of 152 amino acids, such as disclosed in SEQ ID NO: 51. Preferably, the IL-7 variant or mutant presents at least 75%, at least 80%, at least 85%, at least 90%, at least 95%, at least 97%, at least 98% or at least 99% of identity with SEQ ID NO: 51.

Particularly, the at least one mutation occurs at amino acid position 74 and/or 142 of IL-7. Additionally or alternatively, the least one mutation occurs at amino acid positions 2 and 141, 34 and 129, and/or 47 and 92. These positions refer to the position of amino acids set forth in SEQ ID NO: 51.

Particularly, the at least one mutation is an amino acid substitution or a group of amino acid substitutions is selected from the group consisting of C2S-C141S and C47S-C92S, C2S-C141S and C34S-C129S, C47S-C92S and C34S-C129S, W142H, W142F, W142Y, Q11E, Y12F, M17L, Q22E, K81R, D74E, D74Q and D74N or any combination thereof. These mutations refer to the position of amino acid set forth in SEQ ID NO:51. Then, for example, the mutation W142H stands for the substitution of tryptophan of the wth-IL7 into a histidine, to obtain an IL-7m having a histidine in amino acid position 142. Such mutant is for example described under SEQ ID NO: 56.

In one embodiment, the IL-7 variant or mutant comprises sets of substitutions in order to disrupt disulfide bonds between C2 and C141, C47 and C92, and C34-C129. In particular, the IL-7 variant or mutant comprises two sets of substitutions in order to disrupt disulfide bonds between C2 and C141, and C47 and C92; C2 and C141, and C34-C129; or C47 and C92, and C34-C129. For instance, the cysteine residues can be substituted by serine in order to prevent disulfide bonds formation. Accordingly, the amino acid substitutions can be selected from the group consisting of C2S-C141S and C47S-C92S (referred as “SS2”), C2S-C141S and C34S-C129S (referred as “SS1”), and C47S-C92S and C34S-C129S (referred as “SS3”). These mutations refer to the position of amino acids set forth in SEQ ID NO: 51. Such IL-7 variants or mutants are particularly described under the sequence set forth in SEQ ID NOs: 53to 55 (SS1, SS2 and SS3, respectively). Preferably, the IL-7 variant or mutant comprises the amino acids substitutions C2S-C141S and C47S-C92S. Even more preferably, the IL-7 variant or mutant presents the sequence set forth in SEQ ID NO: 54.

In another embodiment, the IL-7 variant or mutant comprises at least one mutation selected from the group consisting of W142H, W142F, and W142Y. Such IL-7 variant or mutant are particularly described in under the sequence set forth in SEQ ID NOs: 57 to 58, respectively. Preferably, the IL-7 variant or mutant comprises the mutation W142H. Even more preferably, the IL-7 variant or mutant presents the sequence set forth in SEQ ID NO: 56.

In another embodiment, the IL-7 variant or mutant comprises at least one mutation selected from the group consisting of D74E, D74Q and D74N, preferably D74E and D74Q. Such IL-7 variant or mutant are particularly described in under the sequence set forth in SEQ ID NOs: 63 to 65, respectively. Preferably, the IL-7 variant or mutant comprises the mutation D74E. Even more preferably, the IL-7 variant or mutant presents the sequence set forth in SEQ ID NO: 63.

In another embodiment, the IL-7 variant or mutant comprises at least one mutation selected from the group consisting of Q11E, Y12F, M17L, Q22E and/or K81R. These mutations refer to the position of amino acids set forth in SEQ ID NO:51. Such IL-7 variant or mutant are particularly described in under the sequence set forth in SEQ ID NOs: 59, 60, 61, 62 and 66, respectively.

In one embodiment, the IL-7 variant or mutant comprises at least one mutation that consists in i) W142H, W142F or W142Y and/or ii) D74E, D74Q or D74N, preferably D74E or D74Q and/or iii) C2S-C141S and C47S-C92S, C2S-C141S and C34S-C129S, or C47S-C92S and C34S-C129S.

In one embodiment, the IL-7 variant or mutant comprises the W142H substitution and at least one mutation consisting of i) D74E, D74Q or D74N, preferably D74E or D74Q and/or ii) C2S-C141S and C47S-C92S, C2S-C141S and C34S-C129S, or C47S-C92S and C34S-C129S.

In one embodiment, the IL-7 variant or mutant comprises the D74E substitution and at least one mutation consisting of i) W142H, W142F or W142Y and/or ii) C2S-C141S and C47S-C92S, C2S-C141S and C34S-C129S, or C47S-C92S and C34S-C129S.

In one embodiment, the IL-7 variant or mutant comprises the mutations C2S-C141S and C47S-C92S and at least one substitution consisting of i) W142H, W142F or W142Y and/or ii) D74E, D74Q or D74N, preferably D74E or D74Q.

In one embodiment, the IL-7 variant or mutant comprises i) D74E and W142H substitutions and ii) the mutations C2S-C141S and C47S-C92S, C2S-C141S and C34S-C129S, or C47S-C92S and C34S-C129S.

The IL IL-7 variant or mutant can comprise its peptide signal or be devoid of it.

In one embodiment, the bifunctional molecule according to the invention comprises an IL-7 variant that comprises or consists of the amino acid sequence set forth in SEQ ID NO: 53-58 or 63-65. Even more preferably, the bifunctional molecule according to the invention comprises an IL-7 variant that comprises or consists of the amino acid sequence set forth in SEQ ID NO: 54, 56 or 63.

Bifunctional Molecule or “Bicki”

The invention particularly provides a bifunctional molecule that comprises or consists in an anti-hPD1 antibody or antibody fragment thereof and IL-7 as disclosed hereabove, the anti-hPD1 antibody or antibody fragment thereof being covalently linked to IL-7, preferably by a peptide linker as disclosed hereabove, particularly as a fusion protein.

Particularly, the bifunctional molecule according to the invention comprises two entities: a first entity comprising or consisting essentially of an anti-hPD1 antibody or fragment thereof; a second entity comprising or consisting essentially of interleukin 7 (IL-7), preferably a human IL-7, these two entities being optionally linked by a peptide linker.

Particularly, the bifunctional molecule according to the invention comprises one, two, three or four molecules of IL-7. Particularly, the bifunctional molecule may comprise only one molecule of IL-7, linked to only one light chain or heavy chain of the anti-PD-1 antibody. The bifunctional molecule may also comprise two molecules of IL-7, linked to either the light or heavy chains of the anti-PD-1 antibody. The bifunctional molecule may also comprise two molecules of IL-7, a first one linked to the light chain of the anti-PD-1 antibody and a second one linked to the heavy chain of the anti-PD-1 antibody. The bifunctional molecule may also comprise three molecules of IL-7, two of them being linked to either the light or heavy chains of the anti-PD-1 antibody and the last one linked to the other chain of the anti-PD-1 antibody. Finally, the bifunctional molecule may also comprise four molecules of IL-7, two molecules linked to the light chains of the anti-PD-1 antibody and two molecules linked to the heavy chains of the anti-PD-1 antibody. Accordingly, the bifunctional molecule comprises between one to four molecules of an immunotherapeutic agent as disclosed herein.

In one embodiment, only one of the light chains comprises one molecule of IL7 (e.g. the bifunctional molecule comprises one molecule of IL7), only one of the heavy chains comprises one molecule of IL7 (e.g. the bifunctional molecule comprises one molecule of IL7), each light chain comprises one molecule of IL-7 (e.g. the bifunctional molecule comprises two molecules of IL7), each heavy chain comprises one molecule of IL-7 (e.g. the bifunctional molecule comprises two molecules of IL7), only one of the light chain and only one of the heavy chain comprises one molecule of immunotherapeutic agent (e.g. the bifunctional molecule comprises two molecules of IL7), each light chain comprises one molecule of IL-7 and only one of the heavy chains comprises one molecule of IL7 (e.g. the bifunctional molecule comprises three molecule of IL7), each heavy chain comprises one molecule of IL-7 and only one of the light chains comprises one molecule of IL7 (e.g. the bifunctional molecule comprises three molecule of IL7), or both light chains and heavy chains comprises one molecule of IL-7 (e.g. the bifunctional molecule comprises four molecules of IL-7).

In one embodiment, the bifunctional molecule according to the invention comprises or consists of:

(a) an anti-human PD-1 antibody or antigen-binding fragment thereof, which comprises (i) a heavy chain, and (ii) a light chain; and

(b) a human interleukin 7 (IL-7) or a fragment or variant thereof, wherein the antibody heavy chain and/or light chain or a fragment thereof is covalently linked to IL-7 by a peptide linker, preferably as a fusion protein.

Preferably, the bifunctional molecule according to the invention comprises or consists of:

(a) a humanized anti-human PD-1 antibody or antigen-binding fragment thereof, which comprises (i) a heavy chain, and (ii) a light chain; and

(b) a human interleukin 7 (IL-7) or a variant or a fragment thereof, wherein the antibody heavy chain or light chain or a fragment thereof is covalently linked to IL-7 by a peptide linker, preferably as a fusion protein.

Preferably, such bifunctional molecule comprises at least one peptide linker connecting the N-terminus of IL-7 to the C-terminus of the heavy chain or of the light chain or both of the anti-human PD-1 antibody, the peptide linker being preferably selected from the group consisting of (GGGGS)₃, (GGGGS)₄, (GGGGS)₂, GGGGS, GGGS, GGG, GGS and (GGGS)₃, even more preferably is (GGGGS)₃.

Preferably, the N-terminal end of IL-7 is connected to the C-terminal end of the heavy chain or of the light chain or both of the anti-human PD-1 antibody, though at least one peptide linker. Alternatively, the C-terminal end of IL-7 is connected to the N-terminal end of the heavy chain or of the light chain or both of the anti-human PD-1 antibody, though at least one peptide linker.

In one embodiment, the bifunctional molecule according to the invention comprises or consists of:

(a) an anti-human PD-1 antibody or antigen-binding fragment thereof, which comprises (i) a heavy chain, and (ii) a light chain,

(b) a human interleukin 7 (IL-7) or a variant or a fragment thereof, and

(c) a peptide linker that connect the N-terminal end of IL-7 to the C-terminal end of the heavy chain or of the light chain or both of the anti-human PD-1 antibody, the peptide linker being preferably selected from the group consisting of (GGGGS)₃, (GGGGS)₄, (GGGGS)₂, GGGGS, GGGS, GGG, GGS and (GGGS)₃, even more preferably is (GGGGS)₃.

In a particular embodiment, the bifunctional molecule according to the invention comprises or consists of:

(a) an anti-human PD-1 antibody or antigen-binding fragment thereof, which comprises:
(i) a heavy chain variable domain comprising HCDR1, HCDR2 and HCDR3, and
(ii) a light chain variable domain comprising LCDR1, LCDR2 and LCDR3,
   wherein:
the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;
the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2;
the heavy chain CDR3 (HCDR3) comprises or consists of an amino acid sequence of SEQ ID NO: 3 wherein either X1 is D or E and X2 is selected from the group consisting of T, H, A, Y, N, E and S, preferably in the group consisting of H, A, Y, N and E; optionally with one, two or three modification(s) selected from substitution(s), addition(s), deletion(s) and any combination thereof at any position but positions 2, 3, 7 and 8 of SEQ ID NO: 3;
the light chain CDR1 (LCDR1) comprises or consists of an amino acid sequence of SEQ ID NO: 12 wherein X is G or T, optionally with one, two or three modification(s) selected from substitution(s), addition(s), deletion(s) and any combination thereof at any position but positions 5, 6, 10, 11 and 16 of SEQ ID NO: 12;
the light chain CDR2 (LCDR2) comprises or consists of an amino acid sequence of SEQ ID NO: 15, optionally with one, two or three modification(s) selected from substitution(s), addition(s), deletion(s) and any combination thereof; and
the light chain CDR3 (LCDR3) comprises or consists of an amino acid sequence of SEQ ID NO:16, optionally with one, two or three modification(s) selected from substitution(s), addition(s), deletion(s) and any combination thereof at any position but positions 1, 4 and 6 of SEQ ID NO: 16; and

(b) a human interleukin 7 of SEQ ID NO: 51 or a variant or a fragment thereof, wherein the antibody heavy chain and/or light chain or a fragment thereof is covalently linked to IL-7 as a fusion protein, preferably by a peptide linker.

In another embodiment, the bifunctional molecule according to the invention comprises or consists of:

(a) an anti-human PD-1 antibody or antigen-binding fragment thereof, which comprises:

(i) a heavy chain variable domain comprising HCDR1, HCDR2 and HCDR3, and

(ii) a light chain variable domain comprising LCDR1, LCDR2 and LCDR3,

wherein:
the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;
the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2;
the heavy chain CDR3 (HCDR3) comprises or consists of an amino acid sequence of SEQ ID NO: 3 wherein either X1 is D and X2 is selected from the group consisting of T, H, A, Y, N, E, preferably in the group consisting of H, A, Y, N, E; or X1 is E and X2 is selected from the group consisting of T, H, A, Y, N, E and S, preferably in the group consisting of H, A, Y, N, E and 5; optionally with one, two or three modification(s) selected from substitution(s), addition(s), deletion(s) and any combination thereof at any position but positions 2, 3, 7 and 8 of SEQ ID NO: 3;
the light chain CDR1 (LCDR1) comprises or consists of an amino acid sequence of SEQ ID NO: 12 wherein X is G or T, optionally with one, two or three modification(s) selected from substitution(s), addition(s), deletion(s) and any combination thereof at any position but positions 5, 6, 10, 11 and 16 of SEQ ID NO: 12;
the light chain CDR2 (LCDR2) comprises or consists of an amino acid sequence of SEQ ID NO: 15, optionally with one, two or three modification(s) selected from substitution(s), addition(s), deletion(s) and any combination thereof; and
the light chain CDR3 (LCDR3) comprises or consists of an amino acid sequence of SEQ ID NO:16, optionally with one, two or three modification(s) selected from substitution(s), addition(s), deletion(s) and any combination thereof at any position but positions 1, 4 and 6 of SEQ ID NO: 16; and

(b) a human interleukin 7 of SEQ ID NO: 51 or a variant or a fragment thereof, wherein the antibody heavy chain or light chain or both or a fragment thereof is covalently linked to IL-7 as a fusion protein, preferably by a peptide linker.

In another embodiment, the bifunctional molecule according to the invention comprises or consists of:
(a) a humanized anti-human PD-1 antibody or antigen-binding fragment thereof, which comprises:
the heavy chain CDR1 (HCDR1) comprises or consists of an amino acid sequence of SEQ ID NO: 1, optionally with one, two or three modification(s) selected from substitution(s), addition(s), deletion(s) and any combination thereof at any position but position 3 of SEQ ID NO: 1;
the heavy chain CDR2 (HCDR2) comprises or consists of an amino acid sequence of SEQ ID NO: 2, optionally with one, two or three modification(s) selected from substitution(s), addition(s), deletion(s) and any combination thereof at any position but positions 13, 14 and 16 of SEQ ID NO: 2;
the heavy chain CDR3 (HCDR3) comprises or consists of an amino acid sequence of SEQ ID NO: 4, 5, 6, 7, 8, 9, 10 or 11 optionally with one, two or three modification(s) selected from substitution(s), addition(s), deletion(s) and any combination thereof at any position but positions 2, 3, 7 and 8 of SEQ ID NO: 4, 5, 6, 7, 8, 9, 10 or 11;
the light chain CDR1 (LCDR1) comprises or consists of an amino acid sequence of SEQ ID NO: 13 or SEQ ID NO:14, optionally with one, two or three modification(s) selected from substitution(s), addition(s), deletion(s) and any combination thereof at any position but positions 5, 6, 10, 11 and 16 of SEQ ID NO: 13 or SEQ ID NO: 14;
the light chain CDR2 (LCDR2) comprises or consists of an amino acid sequence of SEQ ID NO: 15, optionally with one, two or three modification(s) selected from substitution(s), addition(s), deletion(s) and any combination thereof; and
the light chain CDR3 (LCDR3) comprises or consists of an amino acid sequence of SEQ ID NO: 16, optionally with one, two or three modification(s) selected from substitution(s), addition(s), deletion(s) and any combination thereof at any position but positions 1, 4 and 6 of SEQ ID NO: 16; and

(b) a human interleukin 7 of SEQ ID NO: 51 or a variant or a fragment thereof, wherein the antibody heavy chain or light chain or a fragment thereof is covalently linked to IL-7 as a fusion protein, preferably by a peptide linker.

Preferably, the peptide linker is selected from the group consisting of (GGGGS)₃, (GGGGS)₄, (GGGGS)₂, GGGGS, GGGS, GGG, GGS and (GGGS)₃, even more preferably is (GGGGS)₃.

In another embodiment, the invention relates to a bifunctional molecule that comprises:

(a) a humanized anti-hPD1 antibody that comprises:

(i) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;

(ii) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26, and

(b) a human interleukin 7 of SEQ ID NO: 51 or a variant or a fragment thereof,

(c) a peptide linker selected from the group consisting of (GGGGS)₃, (GGGGS)₄, (GGGGS)₂, GGGGS, GGGS, GGG, GGS and (GGGS)₃, even more preferably is (GGGGS)₃, between the light chain and/or the heavy chain of the anti-hPD1 antibody and the human IL-7 or variant or a fragment thereof.

Preferably, the N-terminal end of IL-7 is connected to the C-terminal end of the heavy chain or of the light chain or both of the anti-human PD-1 antibody, though at least one peptide linker. Alternatively, the C-terminal end of IL-7 is connected to the N-terminal end of the heavy chain or of the light chain or both of the anti-human PD-1 antibody, though at least one peptide linker.

In another embodiment, the invention relates to a bifunctional molecule that comprises or consists of:

(a) a humanized anti-hPD1 antibody that comprises:

(i) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;

(ii) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26, and

(b) a human interleukin 7 of SEQ ID NO: 51 or a variant or a fragment thereof, wherein the C-terminal end of the heavy and/or light chain(s) of the antibody or antigen-binding fragment thereof is covalently linked to the N-terminal end of IL-7, preferably by a (GGGGS)₃ peptide linker.

In another embodiment, the invention relates to a bifunctional molecule that comprises or consists of:

a) a humanized anti-h PD1 antibody that comprises:

(i) a heavy chain variable region (VH) comprising or consisting of an amino acid sequence of SEQ ID NO: 18, 19, 20, 21, 22, 23, 24 or 25, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18, 19, 20, 21, 22, 23, 24 or 25, respectively;

(ii) a light chain variable region (VL) comprising or consisting of an amino acid sequence of SEQ ID NO: 27 or SEQ ID NO: 28, optionally with one, two or three modification(s) selected from substitution(s), addition(s), deletion(s) and any combination thereof at any position positions 3, 4, 7, 14, 17, 18, 28, 29, 33, 34, 39, 42, 44, 50, 81, 88, 94, 97, 99 and 105 of SEQ ID NO: 27 or SEQ ID NO: 28.

(b) a human interleukin 7 of SEQ ID NO: 51 or a variant or a fragment thereof,

wherein the C-terminal end of the heavy and/or light chain(s) of the antibody or antigen-binding fragment thereof is covalently linked to the N-terminal end of IL7 to form a fusion protein, preferably by a (GGGGS)₃ peptide linker.

In a preferred embodiment, the C-terminal end of the heavy chain of the antibody or antigen-binding fragment thereof is covalently linked to the N-terminal end of IL-7 to form a fusion protein. Preferably, only the heavy chains of the antibody or antigen-binding fragment thereof are covalently linked to IL-7. In another embodiment, the invention relates to a bifunctional molecule that comprises or consists of:

a) a humanized anti-h PD1 antibody that comprises:

(i) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;

(ii) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26, and

(b) a human interleukin 7 of SEQ ID NO: 51 or a variant or a fragment thereof, wherein the C-terminal end of the heavy chain of the antibody or antigen-binding fragment thereof is covalently linked to the N-terminal end of IL7 to form a fusion protein, preferably by a (GGGGS)₃ peptide linker.

In another embodiment, the invention relates to a bifunctional molecule that comprises or consists of:

(a) a humanized anti-hPD1 antibody that comprises:

(i) a heavy chain variable region (VH) comprising or consisting of an amino acid sequence of SEQ ID NO: 24;

(ii) a light chain variable region (VL) comprising or consisting of an amino acid sequence of SEQ ID NO: 28;

(b) a human interleukin 7 of SEQ ID NO: 51 or a variant or a fragment thereof, wherein the C-terminal end of the heavy chain of the antibody or antigen-binding fragment thereof is covalently linked to the N-terminal end of IL7 to form a fusion protein, preferably by a (GGGGS)₃ peptide linker.

Preferably, the antibody or an antibody fragment thereof has an IgG1 or IgG4 Fc domain.

In one aspect, the antibody or an antibody fragment thereof has an IgG1 Fc domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/I332E; P257I/Q311; K326W/E333S; S239D/I332E/G236A; N297A; L234A/L235A; N297A+M252Y/S254T/T256E; K322A and K444A, preferably selected from the group consisting of N297A optionally in combination with M252Y/S254T/T256E, and L234A/L235, even more preferably an IgG1 Fc domain having the mutation N297A such as described above.

In another aspect, the antibody or an antibody fragment thereof has an IgG4 Fc domain, optionally with a substitution or a combination of substitutions selected from the group consisting of S228P; L234A/L235A, S228P+M252Y/S254T/T256E and K444A, even more preferably an IgG4 Fc domain having the mutation S228P such as described above.

Optionally, in any of the above-specified embodiments, the IL-7 is an IL-7 variant or mutant.

More particularly, the IL-7 variant or mutant presents at least 75% identity with a wild type human IL-7 (wth-IL-7) comprising or consisting of the amino acid sequence set forth in SEQ ID NO: 51, such IL-7 variant comprising at least one amino acid mutation which i) reduces affinity of the IL-7 variant for IL-7 receptor (IL-7R) in comparison to the affinity of wth-IL-7 for IL-7R, and ii) improves pharmacokinetics of the bifunctional molecule comprising the IL-7 variant in comparison with a bifunctional molecule comprising wth-IL-7. More preferably, such mutations i) reduce affinity of the IL-7 variant for IL-7 receptor (IL-7R) in comparison to the affinity of wth-IL-7 for IL-7R, ii) retain the capacity to activate IL-7R; and iii) improve pharmacokinetics of the bifunctional molecule comprising the IL-7 variant in comparison with a bifunctional molecule comprising wth-IL-7.

More specifically, the IL-7 variant or mutant may present at least 75% identity with a wild type human IL-7 (wth-IL-7) comprising or consisting of the amino acid sequence set forth in SEQ ID NO: 51, such IL-7 variant comprising at least one mutation selected from the group consisting of: (i) C2S-C141S and C47S-C92S, C2S-C141S and C34S-C129S, or C47S-C92S and C34S-C129S, (ii) W142H, W142F or W142Y, (iii) D74E, D74Q or D74N, preferably D74E or D74Q; iv) Q11E, Y12F, M17L, Q22E and/or K81R; or any combination thereof.

The IL-7 variant or mutant may comprise at least one set of substitutions selected from the group consisting of C2S-C141S and C47S-C92S (referred as “SS2”), C2S-C141S and C34S-C129S (referred as “SS1”), and C47S-C92S and C34S-C129S (referred as “SS3”). These mutations refer to the position of amino acids set forth in SEQ ID NO:51. Such IL-7 variants or mutants are particularly described under the sequence set forth in SEQ ID Nos :53 to 55 (SS1, SS2 and SS3, respectively). Preferably, the IL-7 variant or mutant comprises the amino acids substitutions C2S-C141S and C47S-C92S. Even more preferably, the IL-7 variant or mutant presents the sequence set forth in SEQ ID NO: 54.

The IL-7 variant or mutant may comprise at least one mutation selected from the group consisting of W142H, W142F, and W142Y. Such IL-7 variant or mutant are particularly described in under the sequence set forth in SEQ ID NOs: 57 to 58, respectively. Preferably, the IL-7 variant or mutant comprises the mutation W142H. Even more preferably, the IL-7 variant or mutant presents the sequence set forth in SEQ ID NO: 56.

The IL-7 variant or mutant may comprise at least one mutation selected from the group consisting of D74E, D74Q and D74N, preferably D74E or D74Q. Such IL-7 variant or mutant are particularly described in under the sequence set forth in SEQ ID NOs: 63 to 65, respectively. Preferably, the IL-7 variant or mutant comprises the mutation D74E. Even more preferably, the IL-7 variant or mutant presents the sequence set forth in SEQ ID NO: 63.

The IL-7 variant or mutant may comprise at least one mutation selected from the group consisting of Q11E, Y12F, M17L, Q22E and/or K81R. These mutations refer to the position of amino acids set forth in SEQ ID NO:51. Such IL-7 variant or mutant are particularly described in under the sequence set forth in SEQ ID NOs: 59, 60, 61, 62 and 66, respectively.

The IL-7 variant or mutant may comprise at least one mutation that consists in i) W142H, W142F or W142Y and/or ii) D74E, D74Q or D74N, preferably D74E or D74Q and/or iii) C2S-C141S and C47S-C92S, C2S-C141S and C34S-C129S, or C47S-C92S and C34S-C129S. The IL-7 variant or mutant may comprise the W142H substitution and at least one mutation consisting of i) D74E, D74Q or D74N, preferably D74E or D74Q and/or ii) C2S-C141S and C47S-C92S, C2S-C141S and C34S-C129S, or C47S-C92S and C34S-C129S.

The IL-7 variant or mutant may comprise the D74E substitution and at least one mutation consisting of i) W142H, W142F or W142Y and/or ii) C2S-C141S and C47S-C92S, C2S-C141S and C34S-C129S, or C47S-C92S and C34S-C129S.

The IL-7 variant or mutant may comprise the mutations C2S-C141S and C47S-C92S and at least one substitution consisting of i) W142H, W142F or W142Y and/or ii) D74E, D74Q or D74N.

The IL-7 variant or mutant may comprise i) D74E and W142H substitutions and ii) the mutations C2S-C141S and C47S-C92S, C2S-C141S and C34S-C129S, or C47S-C92S and C34S-C129S.

The IL-7 variant or mutant may comprise or consist the amino acid sequence set forth in SEQ ID NO: 53, 54, 55, 56, 57, 58, 63, 64 or 65.

In a particular aspect, the IL-7 is an IL-7 variant according to the present invention and the antibody or an antibody fragment thereof has an IgG1 Fc domain, optionally with a substitution or a combination of substitutions selected from the group consisting of T250Q/M428L; M252Y/S254T/T256E +H433K/N434F; E233P/L234V/L235A/G236A+A327G/A330S/P331S; E333A; S239D/A330L/1332E; P257I/Q311; K326W/E333S; S239D/I332E/G236A; N297A; L234A/L235A; N297A +M252Y/S254T/T256E; K322A and K444A, preferably selected from the group consisting of N297A optionally in combination with M252Y/S254T/T256E, and L234A/L235, even more preferably an IgG1 Fc domain having the mutation N297A such as described above. Preferably, the antibody or a fragment thereof is linked to IL-7 or a variant thereof by a linker selected from the group consisting of (GGGGS)₃, (GGGGS)₄, and (GGGS)₃, more preferably by (GGGGS)₃. Preferably, the IL-7 variant comprises a group of amino acid substitutions selected from the group consisting of C2S-C141S and C47S-C92S, C2S-C141S and C34S-C129S, C47S-C92S and C34S-C129S, W142H, W142F, W142Y, D74E, D74Q and D74N. More preferably, the IL-7 variant comprises a group of amino acid substitutions selected from the group consisting of C2S-C141S and C47S-C92S, C2S-C141S and C34S-C129S, W142H, W142F, W142Y, D74E, D74Q and D74N. Still more preferably, the IL-7 variant comprises a group of amino acid substitutions selected from the group consisting of C2S-C141S and C47S-C92S, C2S-C141S and C34S-C129S, W142H and D74E.

In a particular aspect, the bifunctional molecule according to the invention is a fusion protein that comprises or consists of:

(a) an antibody or an antibody fragment thereof such as described hereabove that specifically binds to a target expressed on immune cells surface, preferably T cells, more preferably the target being selected from the group consisting of PD-1, CD28, CD80, CTLA-4, BTLA, TIGIT, CD160, CD4OL, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCRS, CD3, PDL2, CD4 and CD8, preferably of PD-1, TIM3, CD244, LAG-3, BTLA, TIGIT and CD160;

(b) a human interleukin 7 of SEQ ID NO: 51 or a variant or a fragment thereof, and

(c) optionally a peptide linker selected from the group consisting of (GGGGS)₃, (GGGGS)₄, (GGGGS)₂, GGGS, GGG, GGS and (GGGS)₃, preferably (GGGGS)₃.

All and any of the above detailed specific aspects and embodiments disclosed for the bifunctional anti-PD-1 molecules can be applied to these alternative bifunctional molecules.

In a particular aspect, the antibody or an antibody fragment thereof such as described hereabove that specifically binds to a target expressed on immune cells surface, preferably T cells, more preferably the target being selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8, preferably of PD-1, TIM3, CD244, LAG-3, BTLA, TIGIT and CD160; the IL-7 is an IL-7 variant according to the present invention and the antibody or an antibody fragment thereof has an IgG1 Fc domain, optionally with a substitution or a combination of substitutions selected from the group consisting of T250Q/M428L; M252Y/S254T/T256E +H433K/N434F; E233P/L234V/L235A/G236A +A327G/A330S/P331S; E333A; S239D/A330L/1332E; P257I/Q311; K326W/E333S; S239D/I332E/G236A; N297A; L234A/L235A; N297A +M252Y/S254T/T256E; K322A and K444A, preferably selected from the group consisting of N297A optionally in combination with M252Y/S254T/T256E, and L234A/L235, even more preferably an IgG1 Fc domain having the mutation N297A such as described above. Preferably, the antibody or a fragment thereof is linked to IL-7 or a variant thereof by a linker selected from the group consisting of (GGGGS)₃, (GGGGS)₄, and (GGGS)₃, more preferably by (GGGGS)₃. Preferably, the IL-7 variant comprises a group of amino acid substitutions selected from the group consisting of C2S-C141S and C47S-C92S, C2S-C141S and C34S-C129S, C47S-C92S and C34S-C129S, W142H, W142F, W142Y, D74E, D74Q and D74N. More preferably, the IL-7 variant comprises a group of amino acid substitutions selected from the group consisting of C2S-C141S and C47S-C92S, C2S-C141S and C34S-C129S, W142H, W142F, W142Y, D74E, D74Q and D74N. Still more preferably, the IL-7 variant comprises a group of amino acid substitutions selected from the group consisting of C2S-C141S and C47S-C92S, C2S-C141S and C34S-C129S, W142H and D74E.

Binding of the bifunctional molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the anti-hPD-1 antibody/IL-7 complexes can be detected using e.g., an enzyme-linked antibody or antibody fragment which recognizes and specifically binds to IL-7 or to the receptor of IL-7.

In some examples, the bifunctional molecule described herein suppresses the PD-1 signaling pathway by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

Preferably, such bifunctional molecule has the ability to block or inhibit the interaction between PD-1 and its ligand (e.g. PD-L1 and/or PD-L2). In certain embodiments, the bifunctional molecule inhibits the binding interaction between PD-1 and its ligands (e.g. PD-L1 and/or PD-L2) by at least 50%. In certain embodiments, this inhibition may be greater than 60%, greater than 70%, greater than 80%, or greater than 90%.

In some examples, the bifunctional molecule described herein suppresses the PD-1 signaling pathway by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

In some examples, the bifunctional molecule described herein stimulates IFN gamma secretion and/or Alpha4 and Beta7.

In another example, the bifunctional molecule described herein promotes T cell infiltration in tumor.

In some examples, the bifunctional molecule described herein stimulates IL-7R signaling pathway_by at least 10%, at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

In other aspect, the bifunctional molecule described herein retains substantially equivalent biological IL-7 property in comparison to a wild-type IL-7. For instance, it retains comparable biological property as the full-length IL-7 protein. The biological activity of IL-7 protein can be measured using in vitro cellular proliferation assays or by measuring the P-Stat5 into the T cells by ELISA or FACS. Preferably, the IL-7 bifunctional molecule described herein maintains biological activity of at least 10%, 20%, 30%, 40%, 50%, 60% in comparison with the wild type human IL-7, preferably at least 80%, 90%, 95% and even more preferably 99% in comparison with the wild type IL-7. For instance, the biological activity can be assessed by measuring the binding capacity of the bifunctional molecule described herein to IL-7R and/or the capacity to compete with the wild type IL-7 for the binding to IL-7R.

In another example, the bifunctional molecule described herein induce cytokine secretion, and/or proliferation of naïve, partially exhausted and/or fully exhausted T-cell subsets.

Preparation of Bifunctional Molecule—Nucleic Acid Molecules Encoding the Bifunctional Molecule, Recombinant Expression Vectors and Host Cells Comprising Such

To create a bifunctional molecule of the invention, an anti-h PD1 antibody of the invention is functionally linked to IL-7 or a variant thereof.

Both entities of the bifunctional molecule are encoded in the same vector and produced as a fusion protein. Accordingly, also disclosed herein are nucleic acids encoding any of the bifunctional molecule described herein, vectors such as expression vectors or recombinant viruses comprising these nucleic acids, and host cells comprising the nucleic acids and/or vectors. To produce a bifunctional fusion protein which is secreted in stable form by mammalian cells, according to the present invention, nucleic acid sequences coding for the bifunctional molecule are subcloned into an expression vector which is generally used to transfect mammalian cells. General techniques for producing molecules comprising antibody sequences are described in Coligan et al. (eds.), Current protocols in immunology, at pp. 10.19.1-10.19.11 (Wiley Interscience 1992), the contents of which are hereby incorporated by reference and in “Antibody engineering: a practical guide” from W. H. Freeman and Company (1992), in which commentary relevant to production of molecules is dispersed throughout the respective texts.

Generally, such method comprises the following steps of:

(1) transfecting or transforming appropriate host cells with the polynucleotide(s) or its variants encoding the recombinant bifunctional molecule of the invention or the vector containing the polynucleotide(s);

(2) culturing the host cells in an appropriate medium; and

(3) optionally isolating or purifying the protein from the medium or host cells.

The invention further relates to a nucleic acid encoding a bifunctional molecule as disclosed above, a vector, preferably an expression vector, comprising the nucleic acid of the invention, a genetically engineered host cell transformed with the vector of the invention or directly with the sequence encoding the recombinant bifunctional molecule, and a method for producing the protein of the invention by recombinant techniques.

The nucleic acid, the vector and the host cells are more particularly described hereafter.

Nucleic Acid Sequence

The invention also relates to a nucleic acid molecule encoding the bifunctional molecule as defined above or to a group of nucleic acid molecules encoding the bifunctional molecule as defined above.

Antibody DNA sequences can for example be amplified from RNA of cells that synthesize an immunoglobulin, synthesized using PCR with cloned immunoglobulins, or synthesized via oligonucleotides that encode known signal peptide amino acid sequences.

Preferably, the peptide signal comprises or consists of the amino acid sequence of SEQ ID NO: 49 for the VH and/or CH; and/or of the amino acid sequence of SEQ ID NO: 50 for the VL and/or CL. Particularly, the peptide signal is in the N-terminal of the CH, VH, CL and/or VL.

Such nucleic acid may encode an amino acid sequence comprising the VL and/or an amino acid sequence comprising the VH of the antibody (e.g., the light and/or heavy chains of the antibody). Such nucleic acid may be readily isolated and sequenced using conventional procedures.

Particularly, the nucleic acid molecules encoding the bifunctional molecule as defined above comprises:
a first nucleic acid molecule encoding a variable heavy chain domain of an anti-hPD-1 antibody as disclosed herein, optionally with a peptide signal of SEQ ID NO: 49, and
a second nucleic acid molecule encoding a variable light chain domain of an anti-hPD-1 antibody as disclosed herein, optionally with a peptide signal of SEQ ID NO: 50, and
a third nucleic acid encoding IL-7 or a variant thereof, preferably a human IL-7 or a variant thereof, operably linked to either the first nucleic acid or to the second nucleic acid or both, optionally through a nucleic acid encoding a peptide linker.

Preferably, the nucleic acid molecules encoding the bifunctional molecule as defined above comprises:
a first nucleic acid molecule encoding a variable heavy chain domain of SEQ ID NO: 17, wherein X1 is D or E and X2 is selected from the group consisting of T, H, A, Y, N, E and S preferably in the group consisting of H, A, Y, N, and E; optionally with a peptide signal of SEQ ID NO: 49, and
a second nucleic acid molecule encoding a variable light chain domain of SEQ ID NO: 26, wherein X is G or T; optionally with a peptide signal of SEQ ID NO: 50, and
a third nucleic acid molecule encoding human IL-7 of SEQ ID NO: 51, 53, 54, 55, 56, 57, 58, 59, 60, 61, 62, or 63 or a variant or a fragment thereof operably linked to either the first nucleic acid or to the second nucleic acid or both, optionally through a nucleic acid encoding a peptide linker.

Preferably, the nucleic acid molecules encoding the bifunctional molecule as defined above comprises:
a first nucleic acid molecule encoding a variable heavy chain domain of the amino acid sequence set forth in SEQ ID NO: 18, 19, 20, 21, 22, 23, 24 or 25; optionally with a peptide signal of SEQ ID NO: 49, and
a second nucleic acid molecule encoding a variable light chain domain of the amino acid sequence set forth in SEQ ID NO: 27 or SEQ ID NO: 28; optionally with a peptide signal of SEQ ID NO: 50, and
a third nucleic acid molecule encoding human IL-7 of SEQ ID NO: 51, 53, 54, 55, 56, 57, 58, 59, 60, 61, 62, or 63 or a variant thereof operably linked to either the first nucleic acid or to the second nucleic acid or both, optionally through a nucleic acid encoding a peptide linker.

In a very particular embodiment, the nucleic acid molecule encoding a variable heavy chain domain has the sequence set forth in SEQ ID NO: 73 and/or the nucleic acid molecule encoding a variable light chain domain has the sequence set forth in SEQ ID NO: 74.

By operably linked is intended that the nucleic acid encodes a protein fusion including the variable heavy or light chain domain, optionally the peptide linker, and IL-7. Preferably, the linker is selected from the group consisting of (GGGGS)₃, (GGGGS)₄, (GGGGS)₂, GGGGS, GGGS, GGG, GGS and (GGGS)₃, even more preferably is (GGGGS)₃.

In one embodiment, the nucleic acid molecule is an isolated, particularly non-natural, nucleic acid molecule.

The nucleic acid molecule or group of nucleic acid molecules encoding the bifunctional molecule according to the invention is(are) preferably comprised in a vector or a group of vectors.

Vectors

In another aspect, the invention relates to a vector comprising the nucleic acid molecule or the group of nucleic acid molecules as defined above.

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

In one embodiment, both the heavy and light chain coding sequences and/or the constant region of the anti-PD1 antibody are included in one expression vector. Each of the heavy chain coding sequence and the light chain coding sequence may be in operable linkage to a suitable promoter, the heavy chain and/or the light chain being in operable linkage to an immunotherapeutic agent according to the invention. Alternatively, expression of both the heavy chain and the light chain may be driven by the same promoter. In another embodiment, each of the heavy and light chains of the antibody is cloned into an individual vector, one or both of the heavy and light chains, the heavy chain and/or the light chain being in operable linkage to an immunotherapeutic agent according to the invention. In the latter case, the expression vectors encoding the heavy and light chains can be co-transfected into one host cell for expression of both chains, which can be assembled to form intact antibodies either in vivo or in vitro. Alternatively, the expression vector encoding the heavy chain and that encoding the light chain can be introduced into different host cells for expression each of the heavy and light chains, which can then be purified and assembled to form intact antibodies in vitro.

The nucleic acid molecule encoding the humanized anti-PD-1 antibody or antibody fragment thereof can be cloned into a vector by those skilled in the art, and then transformed into host cells. Accordingly, the present invention also provides a recombinant vector, which comprises a nucleic acid molecule encoding the anti-PD-1 antibody or fragment thereof of the present invention. In one preferred embodiment, the expression vector further comprises a promoter and a nucleic acid sequence encoding a secretion signal peptide, and optionally at least one drug-resistance gene for screening.

Suitable expression vectors typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

The methods known to the artisans in the art can be used to construct an expression vector containing the nucleic acid sequence of the bifunctional molecule described herein and appropriate regulatory components for transcription/translation. These methods include in vitro recombinant DNA techniques, DNA synthesis techniques, in vivo recombinant techniques, etc. The DNA sequence is efficiently linked to a proper promoter in the expression vector to direct the synthesis of mRNA. The expression vector may further comprise a ribosome-binding site for initiating the translation, transcription terminator and the like.

An expression vector can be introduced into host cells using a variety of techniques including calcium phosphate transfection, liposome-mediated transfection, electroporation, and the like. Preferably, transfected cells are selected and propagated wherein the expression vector is stably integrated in the host cell genome to produce stable transformants. Techniques for introducing vectors into eukaryotic cells and techniques for selecting stable transformants using a dominant selectable marker are described by Sambrook, by Ausubel, by Bebbington, “Expression of Antibody Genes in Nonlymphoid Mammalian Cells,” in 2 METHODS: A companion to methods in enzymology 136 (1991), and by Murray (ed.), Gene transfer and expression protocols (Humana Press 1991). Suitable cloning vectors are described by Sambrook et al. (eds.), MOLECULAR CLONING: A LABORATORY MANUAL, Second Edition (Cold Spring Harbor Press 1989) (hereafter “Sambrook”); by Ausubel et al. (eds.), CURRENT PROTOCOLS IN MOLECULAR BIOLOGY (Wiley Interscience 1987) (hereafter “Ausubel”); and by Brown (ed.), MOLECULAR BIOLOGY LABFAX (Academic Press 1991).

Host Cells

In another aspect, the invention relates to a host cell comprising a vector or a nucleic acid molecule or group of nucleic acid molecules as defined above, for example for bifunctional molecule production purposes.

As used herein, the term “host cell” is intended to include any individual cell or cell culture that can be or has been recipient of vectors, exogenous nucleic acid molecules, and polynucleotides encoding the antibody construct of the present invention; and/or recipients of the antibody construct or bifunctional molecule itself. The introduction of the respective material into the cell can be carried out by way of transformation, transfection and the like. The term “host cell” is also intended to include progeny or potential progeny of a single cell. Suitable host cells include prokaryotic or eukaryotic cells, and also include but are not limited to bacteria, yeast cells, fungi cells, plant cells, and animal cells such as insect cells and mammalian cells, e.g., murine, rat, rabbit, macaque or human.

In one embodiment, a host cell comprises (e.g., has been transformed with): (1) a vector comprising a nucleic acid that encodes an amino acid sequence comprising the VL of the antibody and/or an amino acid sequence comprising the VH of the antibody and/or the constant region of the antibody, or (2) a first vector comprising a nucleic acid that encodes an amino acid sequence comprising the VL of the antibody and a second vector comprising a nucleic acid that encodes an amino acid sequence comprising the VH of the antibody.

In another embodiment, a host cell comprises (e.g., has been transformed with) a vector comprising both of the entities of the bifunctional molecule. Preferably, a host cell comprises (e.g., has been transformed with) a vector comprising a first nucleic acid molecule encoding a variable heavy chain domain of an anti-hPD-1 antibody as disclosed herein, and a second nucleic acid molecule encoding a variable light chain domain of an anti-hPD-1 antibody as disclosed herein, operably linked to a third nucleic acid encoding IL-7 or a variant or mutant thereof, preferably a human IL-7 or a variant thereof.

A method of humanized anti-PD1 antibody production is also provided herein. The method comprises culturing a host cell comprising a nucleic acid encoding the antibody, as provided above, under conditions suitable for expression of the antibody, and optionally recovering the antibody from the host cell (or host cell culture medium). Particularly, for recombinant production of a humanized anti-PD1 antibody, nucleic acid encoding an antibody, e.g., as described above, is isolated and inserted into one or more vectors for further cloning and/or expression in a host cell.

A bifunctional molecule of the present invention is preferably expressed in eukaryotic cells such as mammalian cells, plant cells, insect cells or yeast cells. Mammalian cells are especially preferred eukaryotic hosts because mammalian cells provide suitable post-translational modifications such as glycosylation. Preferably, such suitable eukaryotic host cell may be fungi such as Pichia pastoris, Saccharomyces cerevisiae, Schizosaccharomyces pombe; insect cell such as Mythimna separate; plant cell such as tobacco, and mammalian cells such as BHK cells, 293 cells, CHO cells, NSO cells and COS cells. Other examples of useful mammalian host cell lines are CV-1 in Origin with SV40 genes cell (COS cell), monkey kidney CV1 line transformed by SV40 (COS-7); human embryonic kidney line (293 or 293 cells as described, e.g., in Graham, F.L. et al, J. Gen Virol. 36 (1977) 59-74); baby hamster kidney cells (BHK); mouse Sertoli cells (TM4 cells as described, e.g., in Mather, J. P., Biol. Reprod. 23 (1980) 243-252); Human

Epithelial Kidney cell (HEK cell); monkey kidney cells (CV1); African green monkey kidney cells (VERO-76); human cervical carcinoma cells (HELA); canine kidney cells (MDCK; buffalo rat liver cells (BRL 3 A); human lung cells (W138); human liver cells (Hep G2); mouse mammary tumor (MMT 060562); TRI cells, as described, e.g., in Mather, J. P. et al, Annals N.Y. Acad. Sci. 383 (1982) 44-68; MRC 5 cells; and FS4 cells.

Other useful mammalian host cell lines include Chinese hamster ovary (CHO) cells, including DHFR″ CHO cells (Urlaub, G. et al, Proc. Natl. Acad. Sci. USA 77 (1980) 4216-220); and myeloma cell lines such as Y0, NSO and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268. For example, mammalian cell lines that are adapted to grow in suspension may be useful.

Particularly, the host cell of the present invention is selected from the group consisting of CHO cell, COS cell, NSO cell, and HEK cell.

For a mammalian host, the transcriptional and translational regulatory signals of the expression vector may be derived from viral sources, such as adenovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

Stable transformants that produce a bifunctional molecule according to the invention can be identified using a variety of methods. After molecule-producing cells have been identified, the host cells are cultured under conditions (e.g. temperature, medium) suitable for their growth and for bifunctional molecule expression. The bifunctional molecules are then isolated and/or purified by any methods known in the art. These methods include, but are not limited to, conventional renaturation treatment, treatment by protein precipitant (such as salt precipitation), centrifugation, cell lysis by osmosis, sonication, supercentrifugation, molecular sieve chromatography or gel chromatography, adsorption chromatography, ion exchange chromatography, HPLC, any other liquid chromatography, and the combination thereof. As described, for example, by Coligan, bifunctional molecule isolation techniques may particularly include affinity chromatography with Protein-A Sepharose, size-exclusion chromatography and ion exchange chromatography. Protein A preferably is used to isolate the bifunctional molecules of the invention.

Pharmaceutical Composition and Method of Administration Thereof

The present invention also relates to a pharmaceutical composition comprising any of the bifunctional molecule described herein, the nucleic acid molecule, the group of nucleic acid molecules, the vector and/or the host cells as described hereabove, preferably as the active ingredient or compound. The formulations can be sterilized and, if desired, mixed with auxiliary agents such as pharmaceutically acceptable carriers and excipients which do not deleteriously interact with the bifunctional molecule of the invention, nucleic acid, vector and/or host cell of the invention. Optionally, the pharmaceutical composition may further comprise an additional therapeutic agent as detailed below.

Preferably, the pharmaceutical compositions of the present invention may comprise a bifunctional molecule as described herein, the nucleic acid molecule, the group of nucleic acid molecules, the vector and/or the host cells as described hereabove in combination with one or more pharmaceutically or physiologically acceptable carriers, diluents, excipients, salt, and anti-oxidant as described hereafter. Desirably, a pharmaceutically acceptable form is employed which does not adversely affect the desired immune potentiating effects of the bifunctional molecule according to the invention. To facilitate administration, the bifunctional molecule as described herein can be made into a pharmaceutical composition for in vivo administration. The means of making such a composition have been described in the art (see, for instance, Remington: The Science and Practice of Pharmacy, Lippincott Williams & Wilkins, 21st edition (2005).

Particularly, the pharmaceutical composition according to the invention can be formulated for any conventional route of administration including a topical, enteral, oral, parenteral, intranasal, intravenous, intramuscular, subcutaneous or intraocular administration and the like. Preferably, the pharmaceutical composition according to the invention is formulated for enteral or parenteral route of administration. Compositions and formulations for parenteral administration may include sterile aqueous solutions that may also contain buffers, diluents and other suitable additives such as, but not limited to, penetration enhancers, carder compounds and other pharmaceutically acceptable carriers or excipients.

The pharmaceutical composition may be prepared by mixing an agent having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A solid pharmaceutically acceptable vehicle may include one or more substances which may also act as flavoring agents, lubricants, solubilizers, suspending agents, dyes, fillers, glidants, compression aids, inert binders, sweeteners, preservatives, dyes, coatings, or tablet- disintegrating agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. Pharmaceutically acceptable carriers include sterile aqueous solutions or dispersions and sterile powders for the extemporaneous preparation of sterile injectable solutions or dispersion. Except insofar as any conventional media or agent is incompatible with the active compound, use thereof in the pharmaceutical compositions of the invention is contemplated.

The bifunctional molecule according to the invention may be dissolved or suspended in a pharmaceutically acceptable liquid vehicle such as water, an organic solvent, ethanol, polyol (for example, glycerol, propylene glycol, and liquid polyethylene glycol, and the like a mixture of both or pharmaceutically acceptable oils or fats and suitable mixtures thereof. The liquid vehicle can contain other suitable pharmaceutical additives such as solubilizers, emulsifiers, buffers, preservatives, sweeteners, flavoring agents, suspending agents, wetting agents, thickening agents, colors, viscosity regulators, stabilizers or osmo-regulators. Suitable examples of liquid vehicles for oral and enteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and peanut oil). For parenteral administration, the vehicle can also be an oily ester such as ethyl oleate and isopropyl myristate. Sterile liquid vehicles are useful in sterile liquid form compositions for enteral administration. The liquid vehicle for pressurized compositions can be a halogenated hydrocarbon or other pharmaceutically acceptable propellant.

The pharmaceutical composition of the invention may further comprise one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline metals or alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetra-acetic acid (EDTA), sorbitol, tartaric acid, phosphoric acid, and the like.

To facilitate delivery, any of the bifunctional molecule or its encoding nucleic acids can be conjugated with a chaperon agent. The chaperon agent can be a naturally occurring substance, such as a protein (e.g., human serum albumin, low-density lipoprotein, or globulin), carbohydrate (e.g., a dextran, pullulan, chitin, chitosan, inulin, cyclodextrin or hyaluronic acid), or lipid. It can also be a recombinant or synthetic molecule, such as a synthetic polymer, e.g., a synthetic polyamino acid. Examples of polyamino acids include polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, and polyphosphazine. In one example, the chaperon agent is a micelle, liposome, nanoparticle, or microsphere. Methods for preparing such a micelle, liposome, nanoparticle, or microsphere are well known in the art. See, e.g., U.S. Pat. Nos. 5,108,921; 5,354,844; 5,416,016; and 5,527,5285.

Pharmaceutical composition typically must be sterile and stable under the conditions of manufacture and storage. The pharmaceutical composition can be formulated as a solution, micro-emulsion, liposome, or other ordered structure suitable to high drug concentration and/or in suitable for injection. The proper fluidity can be maintained, for example, by the use of a coating such as lecithin, by the maintenance of the required particle size in the case of dispersion and by the use of surfactants.

In one embodiment, the pharmaceutical composition is an injectable composition that may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Prolonged absorption of the injectable compositions can be brought about by including in the composition an agent that delays absorption, for example, monostearate salts and gelatin.

Prevention of presence of microorganisms may be ensured both by sterilization procedures, and by the inclusion of various antibacterial and antifungal agents, for example, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

It will be understood by one skilled in the art that the formulations of the invention may be isotonic with human blood that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonicity of the formulation. Tonicity modifiers suitable for this invention include, but are not limited to, saccharides, salts and amino acids.

Pharmaceutical compositions according to the invention may be formulated to release the active ingredients (e.g. the bifunctional molecule of the invention) substantially immediately upon administration or at any predetermined time or time period after administration. The pharmaceutical composition in some aspects can employ time-released, delayed release, and sustained release delivery systems such that the delivery of the composition occurs prior to, and with sufficient time to cause, sensitization of the site to be treated. Means known in the art can be used to prevent or minimize release and absorption of the composition until it reaches the target tissue or organ, or to ensure timed-release of the composition. Such systems can avoid repeated administrations of the composition, thereby increasing convenience to the subject and the physician.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect.

Subject, Regimen and Administration

The present invention relates to a bifunctional molecule as disclosed herein; a nucleic acid or a vector encoding such, a host cell or a pharmaceutical composition, a nucleic acid, a vector or a host cell, for use as a medicament or for use in the treatment of a disease or for administration in a subject or for use as a medicament. It also relates to the use of a pharmaceutical composition, a nucleic acid, a vector or a host cell of the present invention or a bifunctional molecule comprising an anti-PD1 antibody or antibody fragment thereof and IL-7 or a variant thereof in the manufacture of a medicament for treating a disease in a subject. Finally, it relates to a method for treating a disease or a disorder in a subject comprising administering a therapeutically effective amount of a pharmaceutical composition or a bifunctional molecule comprising an anti-PD1 antibody or antibody fragment thereof and IL-7 or a variant thereof to the subject. Examples of treatments are more particularly described hereafter under the section “Methods and Uses”.

The subject to treat may be a human, particularly a human at the prenatal stage, a new-born, a child, an infant, an adolescent or an adult, in particular an adult of at least 30 years old, 40 years old, preferably an adult of at least 50 years old, still more preferably an adult of at least 60 years old, even more preferably an adult of at least 70 years old.

Particularly, the subject is affected with a disease that may involve the PD-1/PD-L1 pathway, particularly wherein, at least one of the ligands of PD-1 (e.g. PD-L1 and/or PD-L2) or PD-1 is/are expressed, especially overexpressed. Preferably, the subject is suffering from cancer, even more preferably from a PD1, PD-L1 and/or PD-L2 positive cancer or a PD-1 positive cancer. Examples of diseases and cancers are more particularly described hereafter under the section “Methods and Uses”.

In a particular embodiment, the subject has already received at least one line of treatment, preferably several lines of treatment, prior to the administration of a bifunctional molecule comprising an anti-PD1 antibody or antibody fragment thereof and IL-7 or a variant thereof according to the invention or of a pharmaceutical composition according to the invention.

Conventional methods, known to those of ordinary skill in the art of medicine, can be used to administer bifunctional molecule or the pharmaceutical composition disclosed herein to the subject, depending upon the type of diseases to be treated or the site of the disease. This composition can be administered via conventional routes, e.g., administered orally, parenterally, enterally, by inhalation spray, topically, rectally, nasally, buccally, vaginally or via an implanted reservoir. The term “parenterally” as used herein includes subcutaneous, intra-cutaneous, intravenous, intramuscular, intra-articular, intra-arterial, intra-synovial, intra-tumoral, intra-sternal, intra-thecal, intra-lesion, and intracranial injection or infusion techniques. When administered parenterally, the pharmaceutical composition according to the invention is preferably administered by intravenous route of administration. When administered enterally, the pharmaceutical composition according to the invention is preferably administered by oral route of administration. This composition can also be administered locally.

The form of the pharmaceutical compositions, the route of administration and the dose of administration of the pharmaceutical composition or the bifunctional molecule according to the invention can be adjusted by the man skilled in the art according to the type and severity of the infection, and to the patient, in particular its age, weight, sex, and general physical condition. The compositions of the present invention may be administered in a number of ways depending upon whether local or systemic treatment is desired.

Preferably, the treatment with the bifunctional molecule or with a pharmaceutical composition according to the invention is administered regularly, preferably between every day, every week or every month, more preferably between every day and every one, two, three or four weeks. In a particular embodiment, the treatment is administered several times a day, preferably 2 or 3 times a day.

The duration of treatment with the bifunctional molecule or with a pharmaceutical composition according to the invention according to the invention is preferably comprised between 1 day and 20 weeks, more preferably between 1 day and 10 weeks, still more preferably between 1 day and 4 weeks, even more preferably between 1 day and 2 weeks. Alternatively, the treatment may last as long as the disease persists.

The bifunctional molecule disclosed herein may be provided at an effective dose range from about 1 ng/kg body weight to about 30 mg/kg body weight, 1 μg/kg to about 20 mg/kg, 10 μg/kg to about 10 mg/kg, or from 100 μg/kg to 5 mg/kg, optionally every one, two, three or four weeks, preferably by parenteral or oral administration, in particular by intravenous or subcutaneous administration.

Particularly, the bifunctional molecule according to the invention can be administered at a subtherapeutic dose. The term “subtherapeutic dose” as used herein refers to a dose that is below the effective monotherapy dosage levels commonly used to treat a disease, or a dose that currently is not typically used for effective monotherapy with anti-hPD1 antibodies.

Methods and Uses

Use in the Treatment of a Disease

The bifunctional molecules, nucleic acids, vectors, host cells, compositions and methods of the present invention have numerous in vitro and in vivo utilities and applications. For example, the bifunctional molecule, the nucleic acids, the vectors, the host cells and/or the pharmaceutical compositions described herein can be used as therapeutic agents, diagnostic agents and medical researches. Particularly, any of the bifunctional molecule, nucleic acid molecule, group of nucleic acid molecules, vector, host cells or pharmaceutical composition provided herein may be used in therapeutic methods and/or for therapeutic purposes. Particularly, the bifunctional molecule, nucleic acid, vector or pharmaceutical composition provided herein may be useful for the treatment of any disease or condition, preferably involving PD-1, such as cancer, autoimmune disease, and infection or other diseases associated with immune deficiency, such as T cell dysfunction. Even more preferably, the invention relates to a method of treatment of a disease and/or disorder selected from the group consisting of a cancer, an infectious disease and a chronic viral infection in a subject in need thereof comprising administering to said subject an effective amount of the bifunctional molecule or pharmaceutical composition as defined above. Examples of such diseases are more particularly described hereafter.

Particularly, the bifunctional molecule according to the invention are called “bifunctional checkpoint inhibitors” as they target both PD-1/PD-L1/PD-L2 and IL7 pathways.

The invention particularly concerns a bifunctional molecule, a nucleic acid, a group of nucleic acids or a vector encoding such, or a pharmaceutical composition comprising such for use in the treatment of a pathology, disease and/or disorder that could be prevented or treated by the inhibition of the binding of PD-L1 and/or PD-L2 to PD-1.

Bifunctional molecules according to the invention target CD127+ immune cells, particularly CD127+ T cells. Such cells may be found in the following areas of particular interest : resident lymphoid cells in the lymph nodes (mainly within paracortex, with occasional cells in follicles), in tonsil (inter-follicular areas), spleen (mainly within the Peri-Arteriolar Lymphoid Sheaths (PALS) of the white pulp and some scattered cells in the red pulp), thymus (primarily in medulla; also in cortex), bone marrow (scattered distribution), in the GALT (Gut Associated-Lymphoid-Tissue, primarily in inter-follicular areas and lamina propria) throughout the digestive tract (stomach, duodenum, jejunum, ileum, cecum colon, rectum), in the MALT (Mucosa-Associated-Lymphoid-Tissue) of the gall bladder. Therefore, the bifunctional molecules of the invention are of particular interest for treating diseases located or involving these areas, in particular cancers.

Accordingly, disclosed herein are methods for treating a disease, in particular associated with the PD-1 and/or PD-1/PD-L1 and/or PD-1/PD-L2 signaling pathway, comprising administering to a subject in need of a treatment an effective amount of any of the bifunctional molecule or pharmaceutical composition described herein. Physiological data of the patient (e.g. age, size, and weight) and the routes of administration have also to be taken into account to determine the appropriate dosage, so as a therapeutically effective amount will be administered to the patient.

In another aspect the bifunctional molecules disclosed herein can be administered to a subject, e.g., in vivo, to enhance immunity, preferably in order to treat a disorder and/or disease. Accordingly, in one aspect, the invention provides a method of modifying an immune response in a subject comprising administering to the subject a bifunctional molecule, nucleic acid, vector or pharmaceutical composition of the invention such that the immune response in the subject is modified. Preferably, the immune response is enhanced, increased, stimulated or up-regulated. The bifunctional molecule or pharmaceutical composition can be used to enhance immune responses such as T cell activation in a subject in need of a treatment. The immune response enhancement can result in the inhibition of the binding of PD-L1 and/or PD-L2 to PD-1 thereby reducing the immunosuppressive environment, stimulating the proliferation and/or the activation of human T-cells and/or the IFNy secretion by human PBMC.

The invention particularly provides a method of enhancing an immune response in a subject, comprising administering to the subject a therapeutic effective amount of any of the bifunctional molecule, nucleic acid, vector or pharmaceutical composition comprising such described herein, such that an immune response in the subject is enhanced.

In some embodiments, the amount of the bifunctional molecule described herein is effective in suppressing the PD-1 signaling (e.g., reducing the PD-1 signaling by at least 20%, 30%, 50%, 80%, 100%, 200%, 400%, or 500% as compared to a control). In other embodiments, the amount of the bifunctional molecule described herein is effective in activating immune responses (e.g., by at least 20%, 30%, 50%, 80%, 100%, 200%, 400%, or 500% as compared to a control).

In some embodiments, the amount of the bifunctional molecule described herein is effective in the inhibition of the binding of human PD-L1 and/or PD-L2 to human PD-1 e.g., inhibiting the binding by at least 20%, 30%, 50%, 80%, 100%, 200%, 400%, or 500% as compared to a control).

In some embodiments, the amount of the bifunctional molecule described herein is sufficient to have an antagonist activity of the binding of human PD-L1 and/or PD-L2 to human PD-1 e.g., inhibiting the binding by at least 20%, 30%, 50%, 80%, 100%, 200%, 400%, or 500% as compared to a control).

The present invention also relates to a bifunctional molecule as described herein; a nucleic acid or a vector encoding such, or a pharmaceutical composition comprising such for use in the treatment of a disorder and/or disease in a subject and/or for use as a medicament or vaccine. It also relates to the use of a bifunctional molecule as described herein; a nucleic acid or a vector encoding such, or a pharmaceutical composition comprising such in the manufacture of a medicament for treating a disease and/or disorder in a subject. Finally, it relates to a method for treating a disease or a disorder in a subject comprising administering a therapeutically effective amount of a pharmaceutical composition or a bifunctional molecule to the subject.

Disclosed herein, are methods of treating a patient with a disease and/or disorder, the method comprising: (a) identifying a patient in need of treatment; and (b) administering to the patient a therapeutically effective amount of any of the bifunctional molecule, nucleic acid, vector or pharmaceutical composition described herein.

A subject in need of a treatment may be a human having, at risk for, or suspected of having a disease associated with the signaling pathway mediated by PD-1. Such a patient can be identified by routine medical examination. For example, a subject suitable for the treatment can be identified by examining whether such subject carries PD-1, PD-L1 and/or PD-L2 positive cells. Preferably, by “PD-L1 positive tumor cells” or “PD-L2 positive tumor cells” is intended to refer to a population of tumor cells in which PD-L1 or PD-L2, respectively, are expressed in at least 10% of tumor cells, preferable at least 20, 30, 40 or 50% of tumor cells.

In one embodiment, a subject who needs a treatment is a patient having, suspected of having, or at risk for a disease, preferably a PD-1, PDL1 and/or PDL2 positive disease, even more preferably a disease where PD-1 and/or at least one ligand of PD-1 is overexpressed. In such subject, the disruption of PD-1/PD-L1 and/or PD-1/PD-L2 interaction thanks to the administration of the bifunctional molecule or pharmaceutical composition according to the invention may enhance immune response of the subject. In some embodiments, any of the humanized anti-PD-1 antibodies or pharmaceutical composition described herein can be used for treating PD-1 positive cells.

Cancer

It is known in the art that blockade of PD-1 by antibodies can enhance the immune response to cancerous cells in a patient. Thus, in one aspect, the invention provides a bifunctional molecule or a pharmaceutical composition for use in the treatment of a subject having a cancer, comprising administering to the individual an effective amount of the bifunctional molecule or pharmaceutical composition, preferably to disrupt or inhibit the PD1/PD-L1 and/or PD-1/PD-L2 interaction and/or to activate IL7 receptor.

In one embodiment, a subject who needs a treatment is a patient having, suspected of having, or at risk for a disease, preferably a PD-1 positive cancer, even more preferably a cancer where PD-1 is expressed or overexpressed. In some embodiments, any of the anti-PD-1 antibodies or pharmaceutical composition described herein can be used for treating PD-1 positive tumor cells. For example, a patient suitable for the treatment can be identified by examining whether such a patient carries PD-1 positive tumor cells. In another embodiment, a subject is a patient having, suspected of having, or at risk for a cancer development, preferably a PD-L1 and/or PD-L2 positive cancer. In some embodiments, any of bifunctional molecule or pharmaceutical composition described herein can be used for treating PD-L1 and/or PD-L2 positive tumors. For example, a human patient suitable for the treatment can be identified by examining whether such a patient carries PD-L1 and/or PD-L2 positive cancer cells.

In further aspects, a bifunctional molecule or pharmaceutical composition for use in treating cancer, preferably a PD-1, PD-L1 and/or PD-L2 positive cancer, even more preferably a cancer wherein PD-1, PD-L1 and/or PD-L2 is/are overexpressed is provided.

In another embodiment, the invention provides the use a bifunctional molecule or pharmaceutical composition as disclosed herein in the manufacture of a medicament for treating a cancer, for instance for inhibiting growth of tumor cells in a subject, preferably PD-1, PD-L1, PD-L2 positive tumor cells. In an aspect of the disclosure, the cancer to be treated is associated with exhausted T cells.

Accordingly, in one embodiment, the invention provides a method of treating a cancer, for instance for inhibiting growth of tumor cells, in a subject, comprising administering to the subject a therapeutically effective amount of bifunctional molecule or pharmaceutical composition according to the invention. Particularly, the present invention relates to the treatment of a subject using a bifunctional molecule such that growth of cancerous cells is inhibited.

Any suitable cancer may be treated with the bifunctional molecule provided herein can be hematopoietic cancer or solid cancer. Such cancers include carcinoma, cervical cancer, colorectal cancer, esophageal cancer, gastric cancer, gastrointestinal cancer, head and neck cancer, kidney cancer, liver cancer, lung cancer, lymphoma, glioma, mesothelioma, melanoma, stomach cancer, urethral cancer environmentally induced cancers and any combinations of said cancers. The present invention is also useful for treatment of metastatic cancers, especially metastatic cancers that express PD-L1 (Iwai et al. (2005) Int. Immunol. 17: 133-144). Additionally, the invention includes refractory or recurrent malignancies.

Preferably, the cancer to be treated or prevented is selected from the group consisting of metastatic or not metastatic, Melanoma , malignant mesothelioma, Non-Small Cell Lung Cancer, Renal Cell Carcinoma, Hodgkin's Lymphoma, Head and Neck Cancer, Urothelial Carcinoma, Colorectal Cancer, Hepatocellular Carcinoma, Small Cell Lung Cancer Metastatic Merkel Cell Carcinoma, Gastric or Gastroesophageal cancers and Cervical Cancer.

In a particular aspect, the cancer is a hematologic malignancy or a solid tumor with high expression of PD-1 and/or PD-L1. Such a cancer can be selected from the group consisting of hematolymphoid neoplasms, angioimmunoblastic T cell lymphoma, myelodysplastic syndrome, acute myeloid leukemia.

In a particular aspect, the cancer is a cancer induced by virus or associated with immunodeficiency. Such a cancer can be selected from the group consisting of Kaposi sarcoma (e.g., associated with Kaposi sarcoma herpes virus); cervical, anal, penile and vulvar squamous cell cancer and oropharyngeal cancers (e.g., associated with human papilloma virus); B cell non-Hodgkin lymphomas (NHL) including diffuse large B-cell lymphoma, Burkitt lymphoma, plasmablastic lymphoma, primary central nervous system lymphoma, HHV-8 primary effusion lymphoma, classic Hodgkin lymphoma, and lymphoproliferative disorders (e.g., associated with Epstein-Barr virus (EBV) and/or Kaposi sarcoma herpes virus); hepatocellular carcinoma (e.g., associated with hepatitis B and/or C viruses); Merkel cell carcinoma (e.g., associated with Merkel cell polyoma virus (MPV)); and cancer associated with human immunodeficiency virus infection (HIV) infection.

Preferred cancers for treatment include cancers typically responsive to immunotherapy. Alternatively, preferred cancers for treatment are cancers non-responsive to immunotherapy.

Preferably, the bifunctional molecules, nucleic acids, vectors, host cells or compositions disclosed herein are for use in the treatment of a subject suffering from cancer with a poor prognosis. As used herein, the term “poor prognosis” refers to a decreased subject survival and/or an early cancer progression and/or an increased or early cancer recurrence and/or an increased risk or occurrence of metastasis. Particularly, the poor prognosis is correlated with a cancer in which a population of Treg cells is present in the tumor or wherein the Treg/Teff ratio is high in the tumor (Chraa et al., 2018 J Leukoc Biol. 2018; 1-13).

By way of example and not wishing to be bound by theory, treatment with an anti-cancer antibody or an anti-cancer immunoconjugate or other current anti-cancer therapy that lead to cancer cell death would potentiate an immune response mediated by PD-1. Accordingly, a treatment of a hyper proliferative disease (e.g., a cancer tumor) may include a bifunctional molecule combined with an anti-cancer treatment, concurrently or sequentially or any combination thereof, which may potentiate an anti-tumor immune responses by the host. Preferably, a bifunctional molecule may be used in combination with other immunogenic agents, standard cancer treatments, or other antibodies.

Infectious Disease

The bifunctional molecule, nucleic acid, group of nucleic acid, vector, host cells or pharmaceutical compositions of the invention are used to treat patients that have been exposed to particular toxins or pathogens. Accordingly, an aspect of the invention provides a method of treating an infectious disease in a subject comprising administering to the subject a bifunctional molecule according to the present invention, or a pharmaceutical composition comprising such, preferably such that the subject is treated for the infectious disease.

Any suitable infection may be treated with a bifunctional molecule, nucleic acid, group of nucleic acid, vector, host cells or pharmaceutical composition according to the present invention provided herein. Some examples of pathogenic viruses causing infections treatable by methods of the invention include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Particularly, the bifunctional molecule or pharmaceutical compositions of the invention are used to treat patients that have chronic viral infection, such infection being caused by viruses selected from the group consisting of Retroviruses, Anellovirus, Circovirus, Herpesvirus, Varicella zoster virus (VZV), Cytomegalovirus (CMV), Epstein-Barr virus (EBV), Polyomavirus BK, Polyomavirus, Adeno-associated virus (AAV), Herpes simplex type 1 (HSV-1), Adenovirus, Herpes simplex type 2 (HSV-2), Kaposi's sarcoma herpesvirus (KSHV), Hepatitis B virus (HBV), GB virus C, Papilloma virus, Hepatitis C virus (HCV), Human immunodeficiency virus (HIV), Hepatitis D virus (HDV), Human T cell leukemia virus type 1 (HTLV1), Xenotropic murine leukemia virus-related virus (XMLV), Rubella virus, German measles, Parvovirus B19, Measles virus, Coxsackie virus.

Some examples of pathogenic bacteria causing infections treatable by methods of the invention include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Some examples of pathogenic parasites causing infections treatable by methods of the invention include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

In all of the above methods, the bifunctional molecule can be combined with other forms of immunotherapy such as cytokine treatment (e.g., interferons, GM-CSF, G-CSF, IL-2), or any therapy, which provides for enhanced presentation of tumor antigens.

Combined Therapy

In particular, bifunctional molecule of the present invention can be combined with some other potential strategies for overcoming immune evasion mechanisms with agents in clinical development or already on the market (see table 1 from Antonia et al. Immuno-oncology combinations: a review of clinical experience and future prospects. Clin. Cancer Res. Off. J. Am. Assoc. Cancer Res. 20, 6258-6268, 2014).

Such combination with the bifunctional molecule according to the invention may be useful notably for:
1—Reversing the inhibition of adaptive immunity (blocking T-cell checkpoint pathways);
2—Switching on adaptive immunity (promoting T-cell costimulatory receptor signaling using agonist molecules, in particular antibodies);
3—Improving the function of innate immune cells;
4—Activating the immune system (potentiating immune-cell effector function), for example through vaccine-based strategies.

Accordingly, also provided herein are combined therapies for any of the diseases associated with the PD-1 signaling as described herein with any of the bifunctional molecule or pharmaceutical composition comprising such, as described herein and a suitable second agent. In an aspect, the bifunctional molecule and the second agent can be present in a pharmaceutical composition as described above. Alternatively, the terms “combination therapy” or “combined therapy”, as used herein, embrace administration of these two agents (e.g., a bifunctional molecule as described herein and an additional or second suitable therapeutic agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route. The agents can be administered by the same route or by different routes. For example, a first agent (e.g., a bifunctional molecule) can be administered orally, and an additional therapeutic agent (e.g., an anti-cancer agent, an anti-infection agent; or an immune modulator) can be administered intravenously. Alternatively, an agent of the combination selected may be administered by intravenous injection while the other agents of the combination may be administered orally.

In another aspect, the invention relates to a therapeutic mean, in particular a combination product mean, which comprises as active ingredients: a bifunctional molecule as defined above and an additional therapeutic agent, wherein said active ingredients are formulated for separate, sequential or combined therapy, in particular for combined or sequential use.

As used herein, the term “sequential” means, unless otherwise specified, characterized by a regular sequence or order, e.g., if a dosage regimen includes the administration of a bifunctional molecule and the second agent, a sequential dosage regimen could include administration of the bifunctional molecule of the invention before, simultaneously, substantially simultaneously, or after administration of the second agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 15 minutes of each other) and intends to embrace joint administration as well as consecutive administration, but if the administration is consecutive it is separated in time for only a short period (e.g., the time it would take a medical practitioner to administer two compounds separately).

It should be appreciated that any combination as described herein may be used in any sequence for treating the disorder or disease described herein. The combinations described herein may be selected on the basis of a number of factors, which include but are not limited to the effectiveness of inhibiting or preventing the target disease progression, the effectiveness for mitigating the side effects of another agent of the combination, or the effectiveness of mitigating symptoms related to the target disease. For example, a combined therapy described herein may reduce any of the side effects associated with each individual members of the combination.

The present invention also relates to a method for treating a disease in a subject comprising administering to said subject a therapeutically effective amount of the bifunctional molecule or the pharmaceutical composition described herein and a therapeutically effective amount of an additional or second therapeutic agent.

When the bifunctional molecule or the pharmaceutical composition described here is co-used with an additional therapeutic agent, a sub-therapeutic dosage of either the bifunctional molecule, the pharmaceutical composition or of the second agent, or a sub-therapeutic dosage of both, can be used in the treatment of a subject, preferably a subject having, or at risk of developing a disease or disorder associated with the cell signaling mediated by PD-1.

Specific examples of additional or second therapeutic agents are provided in WO 2018/053106, pages 36-43.

In an aspect, the additional or second therapeutic agent can be selected in the non-exhaustive list comprising alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents. For instance, the additional therapeutic agent can be selected in the group consisting of chemotherapy, radiotherapy, targeted therapy, antiangiogenic agents, hypomethylating agents, cancer vaccines, epitopes or neoepitopes from tumor antigens, myeloid checkpoints inhibitors, other immunotherapies, and HDAC inhibitors.

In a preferred embodiment, the second therapeutic agent is selected from the group consisting of chemotherapeutic agents, radiotherapy agents, immunotherapeutic agents, cell therapy agents (such as CAR-T cells), antibiotics and probiotics. Said immunotherapeutic agent can also be an antibody targeting tumoral antigen, particularly selected from the group consisting of anti-Her2, anti-EGFR, anti-CD20, anti-CD19, anti-CD52.

In an embodiment, the invention relates to a combined therapy as defined above, wherein the second therapeutic agent is particularly selected from the group consisting of therapeutic vaccines, immune checkpoint blockers or activators, in particular of adaptive immune cells (T and B lymphocytes) and antibody-drug conjugates. Preferably, suitable agents for co-use with any of the anti-hPD-1 antibodies or fragment thereof or with the pharmaceutical composition according to the invention include an antibody binding to a co-stimulatory receptor (e.g., OX40, CD40, ICOS, CD27, HVEM or GITR), an agent that induces immunogenic cell death (e.g., a chemotherapeutic agent, a radio-therapeutic agent, an anti-angiogenic agent, or an agent for targeted therapies), an agent that inhibits a checkpoint molecule (e.g., CTLA4, LAG3, TIM3, B7H3, B7H4, BTLA, or TIGIT), a cancer vaccine, an agent that modifies an immunosuppressive enzyme (e.g., IDO1 or iNOS), an agent that targets T_{reg} cells, an agent for adoptive cell therapy, or an agent that modulates myeloid cells.

In an embodiment, the invention relates to a combined therapy as defined above, wherein the second therapeutic agent is an immune checkpoint blocker or activator of adaptive immune cells (T and B lymphocytes) selected from the group consisting of anti-CTLA4, anti-CD2, anti-CD28, anti-CD40, anti-HVEM, anti-BTLA, anti-CD160, anti-TIGIT, anti-TIM-1/3, anti-LAG-3, anti-2B4, and anti-0X40, anti-CD40 agonist, CD40-L, TLR agonists, anti-ICOS, ICOS-L and B-cell receptor agonists.

In one embodiment, the second therapeutic agent is an antibody targeting tumoral antigen, particularly selected from the group consisting of anti-Her2, anti-EGFR, anti-CD20, anti-CD19, anti-CD52. Combination therapy could also rely on the combination of the administration of bifunctional molecule with surgery, chemotherapy (e.g. such as docetaxel or decarbazine), radiotherapy, immunotherapy (e.g.

such as antibodies targeting CD40, CTLA-4), gene targeting and modulation, and/or other agents such as immune-modulators, angiogenesis inhibitor and any combinations thereof.

Kits

Any of the bifunctional molecules or compositions described herein may be included in a kit provided by the present invention. The present disclosure particularly provides kits for use in enhancing immune responses and/or treating diseases (e.g. cancer and/or infection) associated with the PD-1 signaling or IL-7 signaling.

In the context of the present invention, the term “kit” means two or more components (one of which corresponding to the bifunctional molecule, the nucleic acid molecule, the vector or the cell of the invention) packaged in a container, recipient or otherwise. A kit can hence be described as a set of products and/or utensils that are sufficient to achieve a certain goal, which can be marketed as a single unit.

Particularly, a kit according to the invention may comprise:
a bifunctional molecule as defined above,
an anti-hPD1 antibody or antigen-binding fragment thereof linked to IL-7 or a variant thereof,
a nucleic acid molecule or a group of nucleic acid molecules encoding said bifunctional molecule,
a vector comprising said nucleic acid molecule or group of nucleic acid molecules, and/or
a cell comprising said vector or nucleic acid molecule or group of nucleic acid molecules.

The kit may thus include, in suitable container means, the pharmaceutical composition, and/or the bifunctional molecules, and/or host cells of the present invention, and/or vectors encoding the nucleic acid molecules of the present invention, and/or nucleic acid molecules or related reagents of the present invention. In some embodiments, means of taking a sample from an individual and/or of assaying the sample may be provided. In certain embodiments the kit includes cells, buffers, cell media, vectors, primers, restriction enzymes, salts, and so forth. The kits may also comprise means for containing a sterile, pharmaceutically acceptable buffer and/or other diluent.

The containers may be unit doses, bulk packages (e.g., multi-dose packages) or sub-unit doses. In an embodiment, the invention relates to a kit as defined above for a single-dose administration unit. The kit of the invention may also contain a first recipient comprising a dried/lyophilized bifunctional molecule and a second recipient comprising an aqueous formulation. In certain embodiments of this invention, kits containing single-chambered and multi-chambered pre-filled syringes (e.g., liquid syringes and lyosyringes) are provided.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper penetrable by a hypodermic injection needle). The container may also have a sterile access port (for example the container may be an intravenous solution bag or a vial having a stopper penetrable by a hypodermic injection needle). At least one active agent in the composition is a bifunctional molecule as described herein comprising an anti-hPD1 antibody linked to IL-7 or a variant thereof, or IL-7m.

The compositions comprised in the kit according to the invention may also be formulated into a syringe compatible composition. In this case, the container means may itself be a syringe, pipette, and/or other such like apparatus, from which the formulation may be applied to an infected area of the body, and/or even applied to and/or mixed with the other components of the kit. The components of the kit may alternatively be provided as dried powder(s). When reagents and/or components are provided as a dry powder, a soluble composition can be reconstituted by the addition of a suitable solvent. It is envisioned that the solvent may also be provided in another container means and be suitable for administration. In some embodiments, the kit further includes an additional agent for treating cancer or an infectious disease, and the additional agent may be combined with the bifunctional molecule, or other components of the kit of the present invention or may be provided separately in the kit. Particularly, the kit described herein may include one or more additional therapeutic agents such as those described in the “Combined Therapy” described hereabove. The kit(s) may be tailored to a particular cancer for an individual and comprise respective second cancer therapies for the individual as described hereabove.

The instructions related to the use of the bifunctional molecule or pharmaceutical composition described herein generally include information as to dosage, dosing schedule, route of administration for the intended treatment, means for reconstituting the bifunctional molecule and/or means for diluting the bifunctional molecule of the invention. Instructions supplied in the kits of the invention are typically written instructions on a label or package insert (e.g., a paper sheet included in the kit in the form of a leaflet or instruction manual). In some embodiments, the kit can comprise instructions for use in accordance with any of the methods described herein. The included instructions can comprise a description of administration of the pharmaceutical composition comprising the bifunctional molecule to enhance immune responses and/or to treat a disease as described herein. The kit may further comprise a description of selecting an individual suitable for a treatment based on identifying whether that individual has a disease associated with the PD-1 signaling, e.g., those described herein.

### EXAMPLES

The following Figures and Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 1: Binding of Bicki Anti-hPD1-IL7 Molecules to IL7R and PD1

Affinity assessment by Biacore of recombinant IL-7 cytokine (rIL7) (Biorad PHP046), IL-7 fused to Fc domain (IL7-Fc, CHI-HF-22007 Adipogen), and Bicki anti-PD1 antibodies fused to IL-7 on its heavy (anti-PD1VH-IL7, chimeric form) or light (anti-PD1VL-IL7, chimeric form) chains for CD127 (A) or CD132 (B). CD127 (Sinobiological, 10975-H03H-50) was immobilized onto a CM5 biochip at 20 μg/ml and the indicated protein were added at serial concentrations (0.34; 1.03; 3.11; 9.3; 28 nM). Affinity was analyzed using two state reaction model and bivalent model as IL7-Fc and anti-PD1-IL7 have a dimeric form. To assess affinity of IL-7 to CD132, the complex CD127/IL-7 was performed on the biochip then CD132 receptor (Sinobiological 10555-H08B) was added at different concentration 125, 250, 500, 1000 et 2000 nM.

Blitz method was performed with a Blitz (Forte Bio; USA; reference C22-2 No 61010-1). Recombinant hPD1-His (Sino Biologicals, Beijing, China; reference 10377-H08H) was immobilized at 10 μg/ml by histidine tail into a Ni-NTA biosensor (Forté Bio; USA; reference 18-0029) for 30 seconds. Then, anti-PD1 antibodies were associated at 20 μg/mL for 120 seconds. The dissociation of anti-PD1 antibodies was made in kinetics buffer for 120 seconds. Analysis data was made with the Blitz pro 1.2 software, which calculated association constant (ka) and dissociation constant (kd) and determined the affinity constant KD (ka/kd).

TABLE 1
Binding of Bicki anti-PD1-IL7 to CD127 and CD132 receptors:

| | KD CD127 | KD CD132 |
|---|---|---|
| | (nm) | (nM) |
| IL-7 | 0.7 | 198 |
| anti PD-1 VH IL-7 | 0.9 | 323 |
| anti PD-1 VL IL-7 | 1.2 | 470 |
| IL-7 Fc | 6.3 | 505 |

Affinity assessment by biacore of recombinant IL-7 cytokine, IL-7 fused to Fc domain (IL7-Fc), and anti-PD1 antibody fused to IL-7 on its heavy (anti-PD1VH-IL7) or light (anti-PD1VL-IL7) chains for CD127 (A) or CD132 (B). A steady state affinity model was used for analysis of IL-7 and bivalent model for the fused protein.

TABLE 2
Binding of Bicki anti-PD1-IL7 antibodies to human
recombinant PD-1 protein.

| | Blitz Analysis |
|---|---|
| Anti PD-1 no fusion | 1.079 nm |
| Anti PD-1 VH IL-7 | 1.776 nM |
| Anti PD-1 VL IL-7 | 3.225 nM |
| Anti PD-1 VH VL IL-7 | 1.848 nM |

The inventors observed that fusion of IL-7 to the N-terminal part of the Fc portion (IL7-Fc) decreases affinity to CD127 receptor (Table 1) whereas, unexpectedly, the fusion of IL-7 to the C-terminal part of the Fc region of the heavy chain or the constant domain of the light chain conserves its high affinity for CD127 to a similar extend than rIL-7. These data demonstrate that C-terminal fusion of IL-7 to the anti-PD1 antibody (on heavy or light chain) will be more potent in term of IL-7R activation pathway as compare to a conventional IL7-Fc compound and in term of elimination half-life into the patient body as compare to an IL7 recombinant cytokine for a therapeutic use.

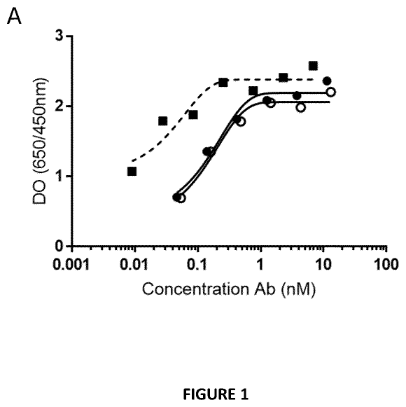

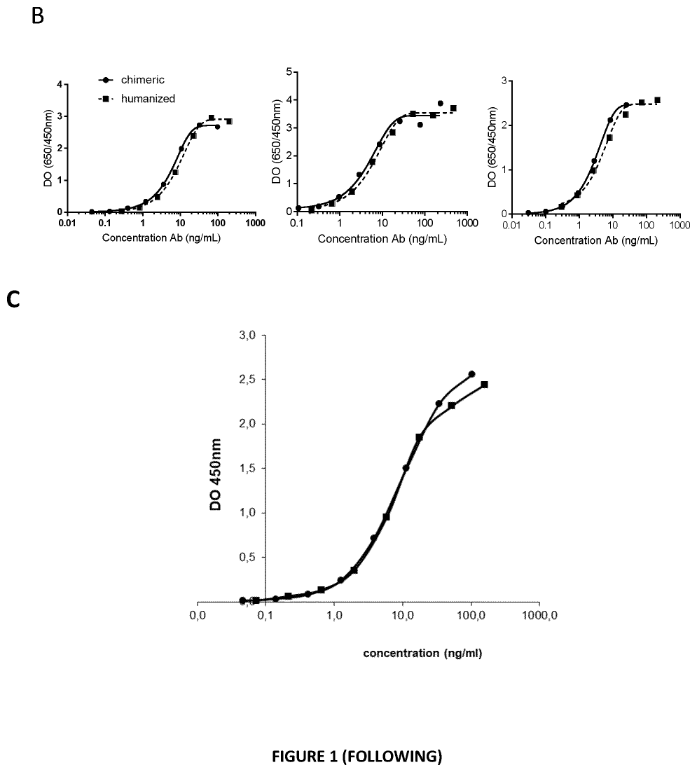

Table 2 and FIGS. 1A and B confirm that Bicki anti-PD1-IL7 molecules (chimeric or humanized) bind to human recombinant PD-1 protein. Humanized form of the anti-PD-1 antibodies bind with similar efficacy than chimeric antibodies to PD-1 recombinant protein. Part A of the FIG. 1A, indicates however a decrease of the binding efficacy of the Bicki compare to an anti-PD1 antibody alone. The inventors have constructed the Bicki IL7 molecules with other anti PD-1 backbones (Pembrolizumab or Nivolumab). FIG. 1C demonstrates that these Bicki molecules conserve good binding to PD-1.

Furthermore, in comparison to IL7-Fc, the bifunctional anti-PD1/IL-7 molecule allows accumulation of IL-7 in PD-1+T cells infiltrates and re-localization of IL-7 on PD-1+T cells.

### Example 2: Antagonistic Capacity of Bicki anti-PD1-IL7 Molecules to block PD-1/PD-L1 and PD1-PDL2 Interactions

PD-L1 was immobilized on Maxisorp plate and the complex anti-PD1 antibody+biotinylated recombinant human PD-1 was added. This complex was generated with a fixed concentration of PD1 (0.6 μg/mL) and different concentrations of anti-PD-1 antibody were tested. Chimeric forms of PD-1 antibodies were used in this test. Revelation was performed with streptavidin peroxidase to detect biotinylated PD1 molecule and revealed by colorimetry at 450 nm using TMB substrate. Affinity assessment by Biacore of PD-1 recombinant protein pre-incubated with anti-PD1 antibody, anti-PD1VH-IL7 or anti-PD1VL-IL7 antibodies on human PD-L2 recombinant protein. Human recombinant PD-L2 is immobilized on the CM5 biochip and the complex antibody (200nM)+recombinant human PD-1 (100 nM) was added. Data are represented in % of relative response of interaction as measured by Biacore: 100%=PD-1 relative response. Chimeric forms of the PD-1 antibodies were used in this experiment.

Results

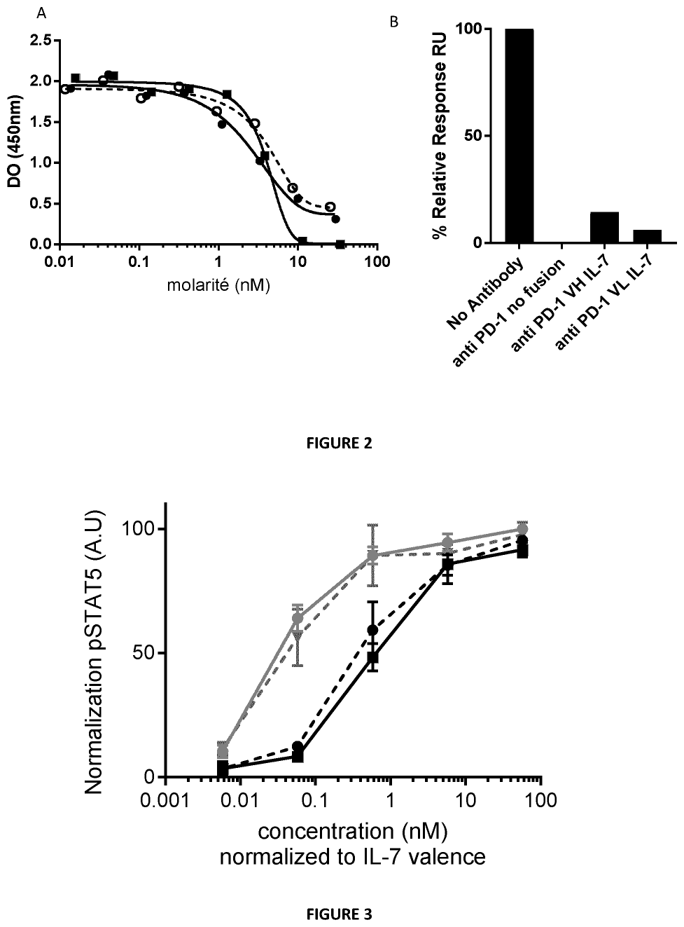

As shown in FIG. 2, Bicki anti-PD1-IL7 molecules present a very good capacity to block interaction of PD1 to PD-L1 as well as to PD-L2. While anti-PD1 alone presented the highest binding to PD1 as compare to the Bicki molecule in ELISA assay, unexpectedly the inhibition of the interaction of PD1-PDL1 or PD-1-PDL2 is comparable between the antibody and Bicki format confirming that Bicki anti-PD1-IL7 molecule of the invention could be as potent as an anti-PD1 antibody.

### Example 3: Ex Vivo, IL-7R Signaling Pathway Analysis on Human PBMCs After Stimulation with Bicki Anti-PD1-IL7 Molecules

PBMCs isolated from peripheral blood of human healthy volunteers were incubated 15 minutes with recombinant IL-7, Bicki anti-PD1-IL-7 (PD-1VH-IL7/PD1VL-IL7). Cells were then fixed, permeabilized and stained with an AF647 labeled anti-pSTAT5 (clone 47/Stat5(pY694)). Data were obtained by calculating MFI pSTAT*%pSTAT5+ population and normalized (100%=rIL-7 57.5 nM) and represent the mean of 3 different donors in two independent experiments.

Results

After analysis of the binding to IL7R of the Bicki anti-PD1-IL7 molecules, the IL7R activation was measured by flow cytometry on STAT5phosphorylation in total human PBMCs or on CD4+ and CD8+ T cells.

FIG. 3 shows that STATS phosphorylation was induced similarly with the Bicki anti-PD1-IL7 molecules fused on heavy or light chain. The maximal activity of the Bicki anti-PD1-IL7 molecules was similar to recombinant IL-7 alone with an EC50 around 0.7 nM.

### Example 4: In Vitro and Ex Vivo Analysis of T Cell Activation and Proliferation Treated with Bicki anti-PD1-IL7 Molecules

A cell-based assay, a Discover'x PD-1 Path Hunter Bioassay kit, was performed. Jurkat T cells stably expressing an engineered PD-1 receptor fused to Beta-gal fragment (ED) and an engineered SHP1 fused to complementing Beta-gal fragment (EA) was used in co-culture of Jurkat cells with PD-L1 expressing cells results in PD-1 phosphorylation and recruitment of engineered SHP-1 forcing complementation of the ED and EA fragment and generation of an active Beta-gal enzyme. After substrate addition, the Beta Gal enzyme generates a chemiluminescence signal that is proportional to PD-1 signaling activation. The addition of anti PD-1 antibodies blocks PD-1 signaling leading to a loss of bioluminescence signal (RLU). Anti PD-1 antibody or Bicki anti-PD1-IL7 molecules were tested at different molar concentrations.

A promega PD-1/PD-L1 bioassay kit was performed. Two cell lines were used (1) Effector T cells (Jurkat stably expressing PD-1, NFAT-induced luciferase) and (2) activating target cells (CHO K1 cells stably expressing PDL1 and surface protein designed to activate cognate TCRs in an antigen-independent manner). When cells are cocultured, PD-L1 /PD-1 interaction directly inhibits TCR mediated activation thereby blocking NFAT activation and luciferase activity. The addition of an anti-PD1 antibody blocks the inhibitory signal leading to NFAT activation and luciferase synthesis. After adding BIOGLO luciferin, Luminescence is quantified and reflects T cell activation. Serial molar concentrations of anti-PD1 antibody or Bicki anti-PD1-IL7 antibodies were tested.

Human PBMCs cells isolated from peripheral blood of healthy volunteers were stimulated with anti CD3/CD28 coated plate (clone OKT3 and CD28.2 respectively, 3 μg/mL) to induce PD-1 expression. Twenty after stimulation, PBMCs were harvested and restimulated on OKT3/PDL1 coated plate (2 and 5 μg/mL, respectively) in the presence of recombinant IL7 (rIL-7) or anti-PD1 antibody fused to IL-7 on the heavy chain (anti —PD-1 VH IL-7) or light chain (anti-PD-1 VL IL-7). Chimeric form of the Bicki anti-PD1-IL7 antibodies were used in this test at a fixed dose (5 μg/ml) of antibody or at multiple doses. Day 5 following stimulation, T cell proliferation was assessed by 3H thymidine incorporation and secreted IFN-γ was dosed by sandwich ELISA.

Results

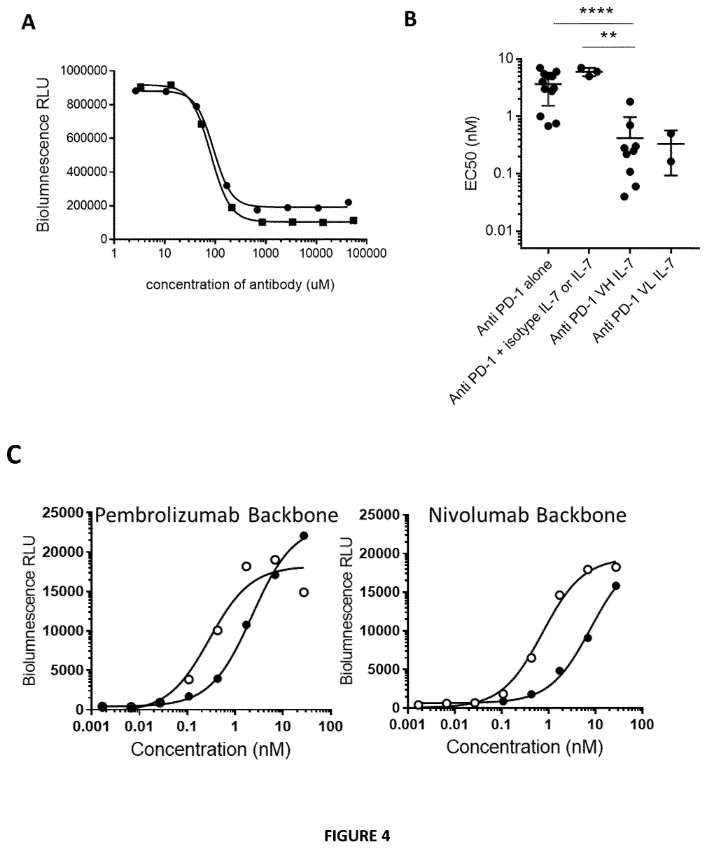

To determine the capacity of Bicki anti-PD1-IL7 molecules to block PD-1 signaling in cell-based assay, a Discover'x PD-1 Path Hunter Bioassay kit was performed. Results presented FIG. 4A, show that the Bicki anti-PD1VH-IL7 is able to inhibits 50% of the SHP1 activation at a concentration of 92.1 μM. This result is quite similar with the result obtained with an anti-PD1 alone which is 80.66 μM, showing a good inhibitory efficacy of PD1/PDL1 interaction leading to a PD1 pathway inhibition. In parallel, the inhibition of the inhibitory signal induced by the interaction of PD1 with PDL1 at T cell surface was measured using the NFAT bioassay. FIG. 4B presents the EC50 of each antibody or therapeutic combination tested. In a surprising manner, the inventors observed that the EC50 of both Bicki anti-PD1-IL7 molecules tested is significantly better than anti-PD1 or anti-PD1+rIL7 combination (mean of 0.41 and 0.33 nM for Bicki VH-IL7 and VL-IL7 versus 3.6 and 6 nM for anti-PD1 and anti-PD1+rIL7). Unexpectedly, this result highlights that Bicki anti-PD1-IL7 is more efficient to induce T cell activation on PD-1+ T lymphocytes than the combination of anti-PD1+IL-7, demonstrating a synergistic effect of the Bicki molecules on PD1+T cells. FIG. 4C demonstrates that these Bicki IL7 molecules constructed with pembrolizumab and nivolumab possess similar synergistic effect compared to the anti PD-1 alone, suggesting that the invention can be suitable with other anti PD-1 backbones.

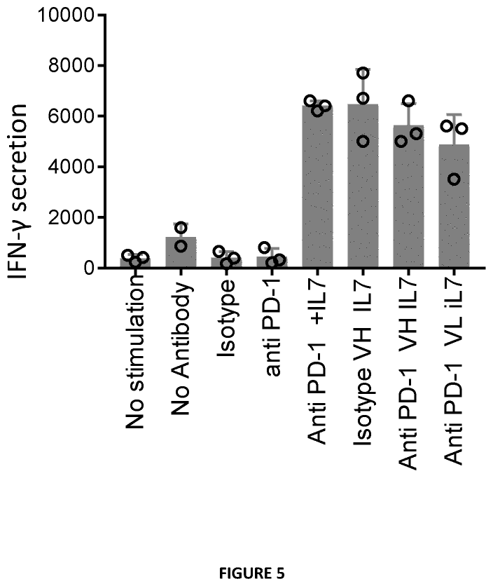

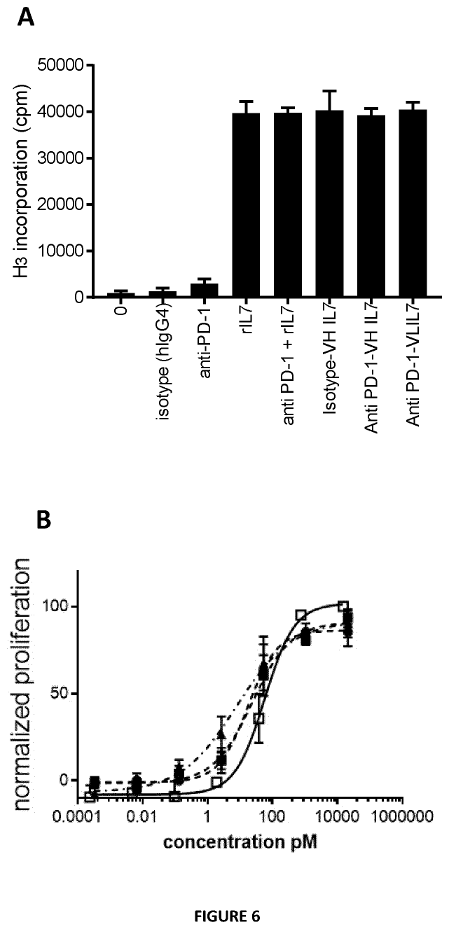

In parallel, ex vivo T cell activation and proliferation was tested following treatment with anti-PD1 antibody, rIL7 or anti-PD1+rIL7 or Bicki anti-PD1-IL7 molecules. Results presented FIGS. 5 and 6 show that IL7 fused to an anti-PD1 is able to induce IFNg secretion and to induce T cell proliferation in a manner comparable to IL7 cytokine alone. However, FIG. 6B shows a better efficacy of the Bicki molecule to induce T cell proliferation by presenting an EC50 of 20 pM compare to IL7 cytokine with 50 pM.

### Example 5: Integrin Expression at Cell Surface Following Bicki Anti-PD1-IL7 Molecules Rreatment

Human PBMCs were incubated 3 days with IL-7 (50 ng/mL) or anti PD-1 or anti-PD1VH-IL7 (5 μg/mL) and stained for Alpha 4 (BDbiosciences, Rungis, France, reference 559881), Beta 7 (BDBiosciences, Reference 555945) or LFA-1 integrin(CD11a/CD18) (BDBiosciences, CD11a reference 555380 and CD18 reference 557156). FACS was analyzed by LSR and are represented in median fluorescence for each marker. Data represent 3 independents experiments with 3 different donors.

Results

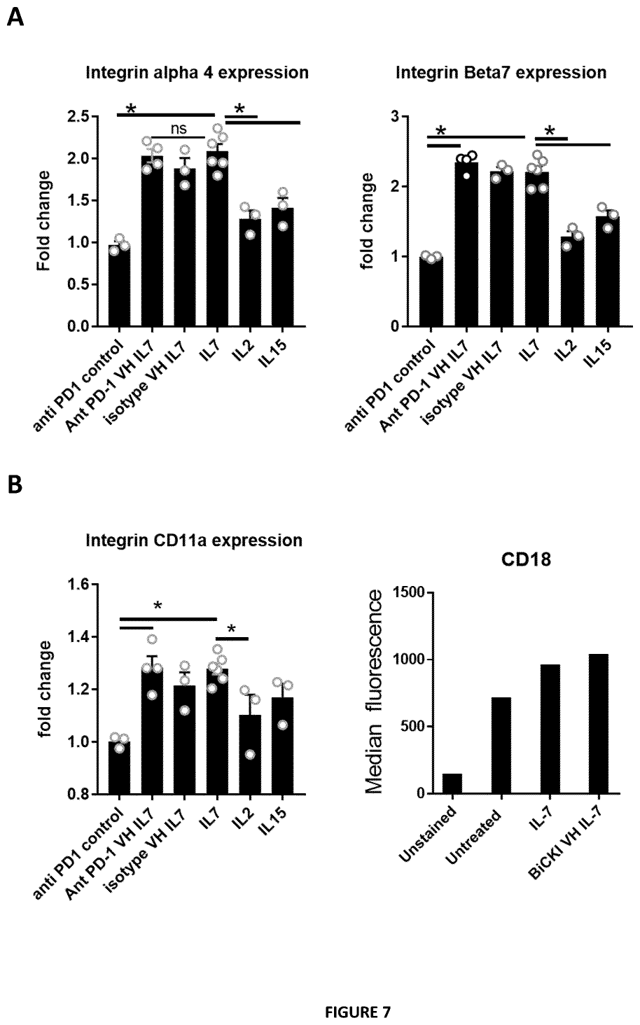

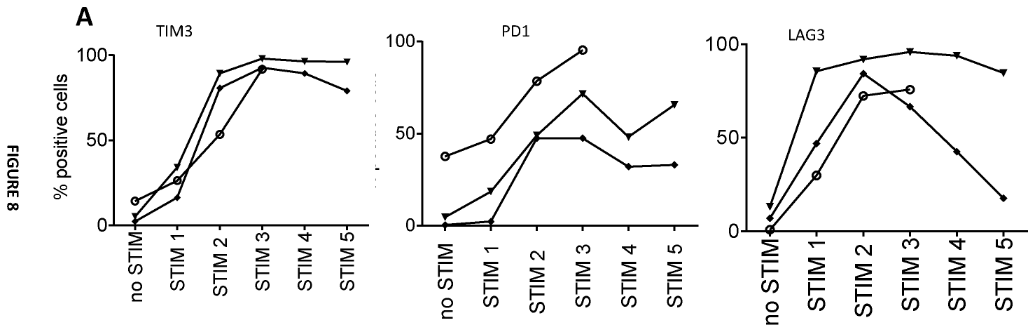

The inventors show FIG. 7 that bicki anti-PD1-IL7 molecules stimulate overexpression of some integrins such as Alpha4 and Beta7 gut homing integrins and LFA1 integrin (CD11a and CD18) as good as rIL7 In comparison, IL-2 and IL-5 cytokine did not significantly modify the expression of these integrins (a4, Beta7) or at least to significantly lower extent to IL-7 and BiCki anti-PD-1 IL-7. Those data supported the interest of Bicki anti-PD1-IL7 molecules for the treatment of PD-1 resistant colorectal cancer because it demonstrates its capacity to promote T cell infiltration into the tumor site via IL-7. Through binding to its ligand ICAM-1 on endothelial cells, LFA-1 mediates T cell trafficking and extravasation in inflamed tissue. Other studies demonstrated that IL-7 stimulates expression of LFA-1 and VLA-4 adhesion molecules promoting transmigration of T cells into any inflamed tissue. This data support that Anti PD-1/IL-7 bifunctional molecule may promote T-cell infiltration in multiple cancer subtypes. Lack of T cell infiltration in the tumor site is nowadays the major obstacle to anti-PD1 efficacy.

### Example 6: Proliferation and Activation of Naïve, Partially Exhausted and Fully Exhausted T-Cell Subsets Treated with Bicki Anti-PD1-IL7 Molecules

Chronic Antigen Stimulation of T Cells Leading to Exhaustion.

Human PBMCs were repeatedly stimulated on CD3 CD28 coated plate (3 μg/mL of OKT3 and 3 μg/mL CD28.2 antibody) every 3 days. Twenty-four hours following stimulation, T cells were stained for PD-1, Lag3 and Tim 3 inhibitory receptors to analyze their exhaustion states after each stimulation. Expression was analyzed by flow cytometry using fluorochrome labeled antibody and FACS LSRII. Twenty-four hours following each stimulation, T cell were restimulated on CD3/PD-L2 coated plate, proliferation capacity was determined by thymidine 3H incorporation Day 5 following each stimulation and IFNg secretion was analyzed into the supernatant by ELISA. Response of exhausted T cells to IL-7 and Bicki anti-PD1-IL7 molecules was analyzed by STATS phosphorylation 48h after each stimulation.

T cells were incubated 15 minutes with serial dilution (starting at 29 nM to 0.29 fM of recombinant IL-7, anti-PD-1 fused to IL-7 (PD-1 VH IL-7 / PD-1 VL IL-7) or isotype control fused to IL-7 (B12 VH IL-7). Cells were then fixed, permeabilized and stained with an AF647 labeled anti-pSTAT5 (clone 47/Stat5(pY694)).

The percentage of pSTAT5+cells was analyzed following treatment with 29 nM of IL-7 or Bicki anti-PD1VH-IL7 molecules after each stimulation.

Human PBMCs were repeatedly stimulated on CD3 CD28 coated plate (3 μg/mL of OKT3 and 3 μg/mL CD28.2 antibody). Twenty-four hours after each stimulation, T cells were restimulated on OKT3 coated plate (2 μg/mL) in the presence of anti PD-1, IL-7 or anti PD-1 fused to IL-7 (Anti D-1 VH IL7 or anti PD-1 VL IL-7). H3 incorporation assay was performed on Day 5 to determine T cell proliferation. T cell stimulated 3 times were re-stimulated on anti-CD3, anti CD3+ recombinant PDL1 or anti CD3 +recombinant PDL2 coated plate (2 and 5 μg/mL respectively). H3 incorporation assay was performed on Day 5.

Results

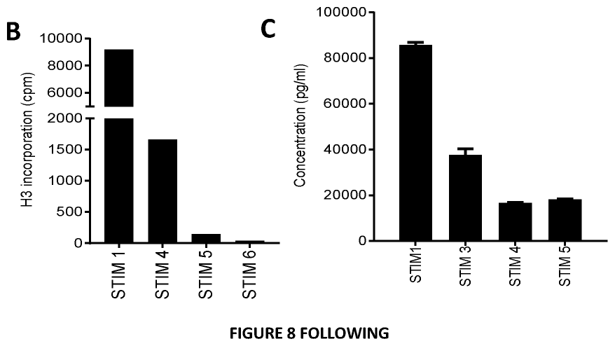

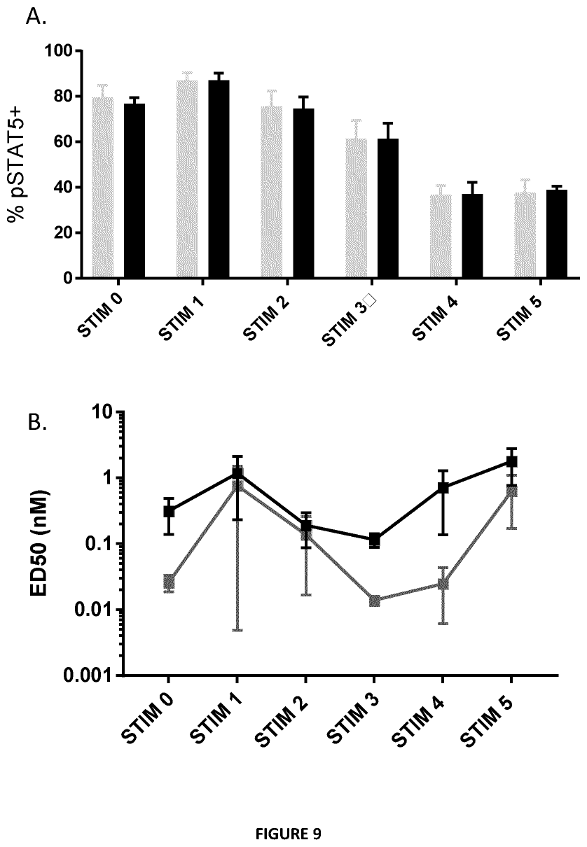

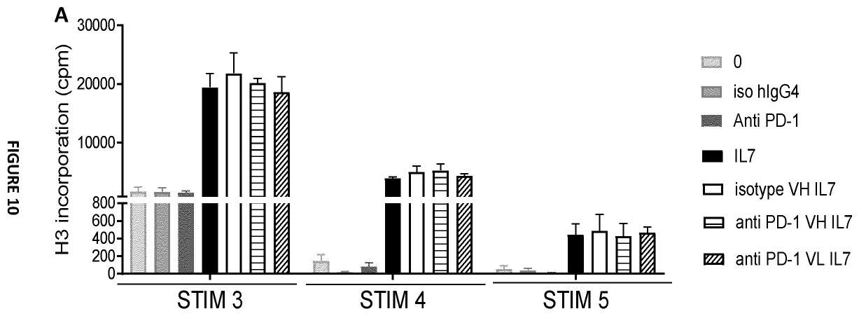

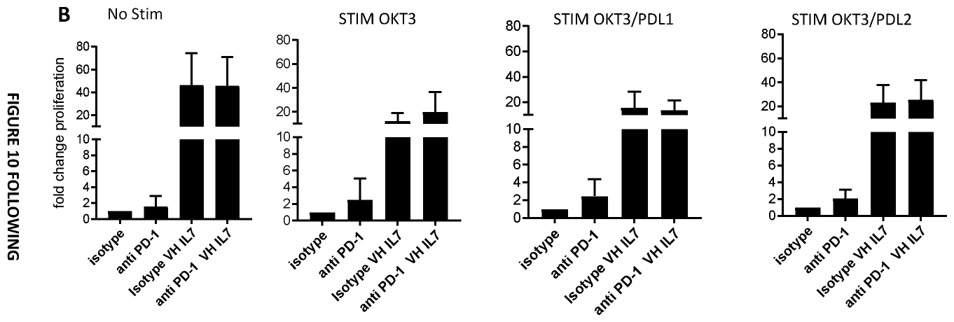

Using a model of repeated TCR stimulation in vitro, the inventors recapitulated chronic antigen stimulation of T cells such as in the context of immunogenic tumors and characterized capacity of T cells to respond to IL-7 after chronic antigen stimulation-induced T-cell exhaustion (FIGS. 8 and 9). In this model, T cells highly express inhibitory receptor (Tim 3, PD1, Lag3) over-stimulation and loss their capacity to proliferate and to secrete cytokines, a key characteristic of exhausted T cells (FIG. 8). In contrast to what was previously published in the literature or predicted based on strong decreased of IL7R expression on exhausted T-cells, the inventors observed that partially and fully exhausted human T cells still respond to IL-7 as shown by pSTAT5 activation (FIG. 9), and that IL-7 increases capacity of T cell proliferation even when T cells are fully exhausted (5 stimulations) (FIG. 10). However, sensitivity to IL-7 decreases along with repeated stimulations as the amount of IL-7 required to activate T cells increases (FIG. 9B and FIGS. 10A and B). These data justify the need of high IL-7 concentration into the tumor microenvironment to activate chronically stimulated exhausted T cells. Such high local concentration of IL-7 could be reached by increasing IL-7 half-life for example by fusing with an antibody or Fc fragment recombinant protein. Another advantage of fusing IL-7 to a monoclonal antibody compared to an Fc-domain (IL7-Fc) is that targeting PD1 with an anti-PD1 antibody of the invention will induce a specific localization of IL-7 cytokine closed to or directly on intratumoral PD-1+exhausted T cells, exactly on the cells that require higher concentration of IL-7. Furthermore, as PD-1+T cells accumulates into the tumor micro-environment, the Bicki anti-PD1-IL7 molecules will lead to increase local concentration of IL-7 where PD-1+cells are accumulated.

### Example 7: Ex Vivo Analysis of Treg Suppressive Activity on Effector T Cells

CD8+effector T cells and CD4+ CD25high CD127low Treg were isolated from peripheral blood of healthy donor, stained with cell proliferation dye (CPDe450 for CD8+ T cells). Treg/CD8+Teff were then co-cultured at ratio 1:1 on OKT3 coated plate (2 μg/mL) in presence or absence of rIL-7 (10 ng/mL, 0.58 nM), anti-PD1 (0.58 nM), anti-PD1+rIL-7(0.58 nM, bicki anti-PD1VH-IL7 (0.58 nM) for 5 days. Proliferation of effector T cells were analyzed by cytofluorometry.

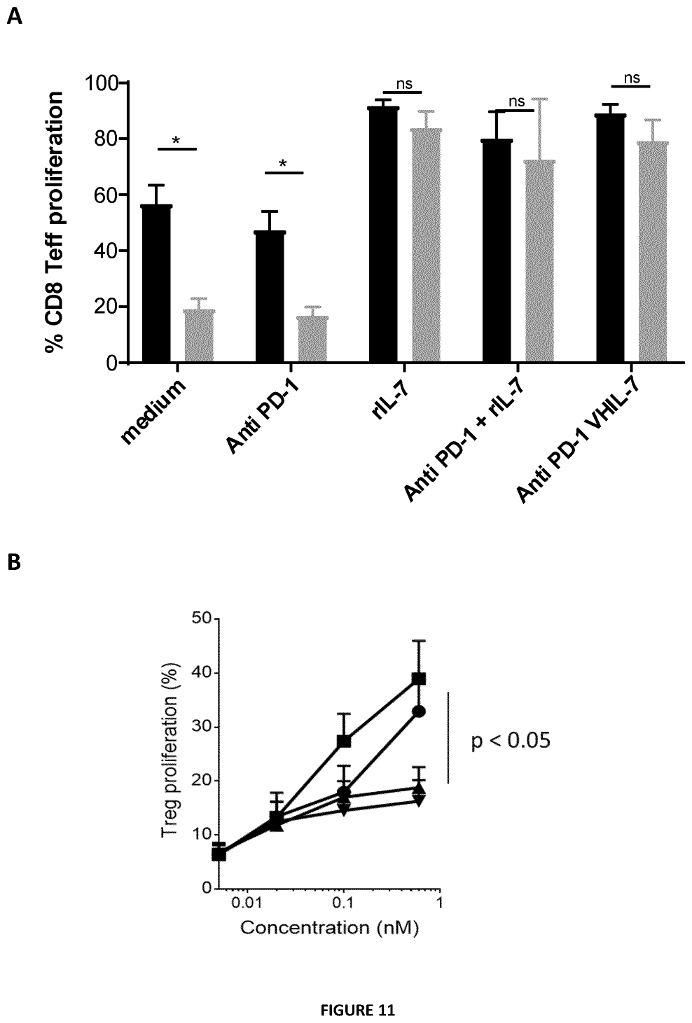

Although anti-PD1 therapy stimulates T cell effector functions, immunosuppressive molecules (TGFB, IDO, IL-10 . . . ) and regulatory cells (Treg, MDSCs, M2 macrophages) create a hostile microenvironment that limits full potential of the therapy. Then the sensitivity of intratumoral T reg response to IL-7 treatment was tested by measuring the proliferation of effector T cells (FIG. 11). Although Treg cells express a low level of IL-7R (CD127), they are still able to stimulate pSTAT5 following IL-7 treatment. In a suppressive assay, by coculturing Treg and T effector cells, the inventors observed FIG. 11 that IL-7 or bicki anti-PD1-IL7 treatment blocks Treg mediated inhibitory effect. The anti-PD1 antibody is not able to inhibit Treg suppressive activity on T effector cells. The IL7 is known to disarm Treg suppressive functions (Allgäuer A, et al. J. Immunol. 2015). Inventors show FIG. 11, that Bicki anti-PD1-IL7 molecules disarm Treg mediated inhibition leading to proliferation of effector T cells in as good as IL7 cytokine. Although Treg cells express low level of IL-7 receptor, it is well-described that IL-7 directly affects Treg and abrogates their suppressive function(Liu W, et al. J Exp Med. 2006 Jul. 10; Liu W et al.; Seddiki N, et al. J exp Med 2006 Jul. 10; Codarri L, et al. 2007; Heninger A K, et al. J immuonl 2012 Dec. 15). Moreover, FIG. 11B shows that targeting IL-7 signaling should hold greater promise compared to IL-2 and IL-15 signaling as IL-2 and IL-15 strongly stimulate proliferation of both Tregs as opposed to IL-7 and Bicki anti PD-1/IL-7. The inventors show in that experiment that Bicki anti-PD1-IL7 molecules will favor the T cell effector over T regulatory immune balance by stimulating effector T-cell proliferation and survival while sparing regulatory T cells. Targeting IL-7 signaling should hold greater promise compare to IL-2 signaling as IL-2 acts on both Treg and T effector cells whereas IL-7 selectively activates T effector cells.

### Example 8: Efficacy of Bicki Anti-PD1-IL7 Molecules in a Humanized Mouse Model and Ex Vivo on Human T-Cells From Tumors or Ex-Vivo Human Tumor Explant Cultures

Humanized Mouse Model:

1^{e}6 human PBMCs were intraperitoneally injected into the mice. Mice were treated twice a weekly with anti-PD1 antibody or Bicki anti-PD1VH-IL7 molecule (5 mg/kg). Day 16 after injection blood was harvested and mice sacrificed. Percentage of human CD3 T cells was analyzed by flow cytometry in human CD45 cells, human IFN gamma was dosed in the plasma by ELISA, and infiltration of human CD3+ cells was quantified in the colon of the mice by immune-histofluorescence. Proximal and distal colon, Liver and Lungs were embedded in TISSUE TEK OCT. Slices were stained for Dapi and human CD3. For liver and lung, CD3 infiltration was quantified in pixel²/mm². For colon, CD3+ infiltrated T cells were counted.

Ex Vivo T Cell Study From Different Cancers:

T cells were extracted from kidney cancer, metastatic colorectal cancer, hepatocellular carcinoma, schwannoma biopsies and stained for CD3, CD4, CD8, PD-1, CD127 and CD132. Immunofluorescence was analyzed by FACS LSRII. CD4+CD3+ or CD8+CD3+ populations were analyzed.

Cells were treated 15 minutes with recombinant IL-7 or anti-PD1VH-IL7 antibody (29 nM). Cells were then fixed, permeabilized and stained for pSTAT5 (clone 47/Stat5(pY694)), CD3, CD4 and CD8. Cells were then washed by centrifugation and resuspended in complete media with an isotype control, anti PD-1, an B12 isotype fused to II-7 (isotype-VH IL-7) or with anti PD-1 fused to IL-7 (anti-PD-1 VH IL-7) at a concentration of 5 μg/mL. After 48 hours, supernatant was harvested and IFNγ secretion was dosed using MSD technology (Meso scale Discovery).

Intratumoral FoxP3 Treg staining in CD3+ T cell population. Facs analysis of pSTAT5 in FoxP3-CD3+ effector T cell versus FoxP3-CD3+ Treg cells after treatment with recombinant IL-7 or anti PD-1 VHIL7 (29 nM) (15 min incubation). Cells were then fixed, permeabilized and stained for pSTAT5 (clone 47/Stat5(pY694)), CD3 and Foxp3.

Results

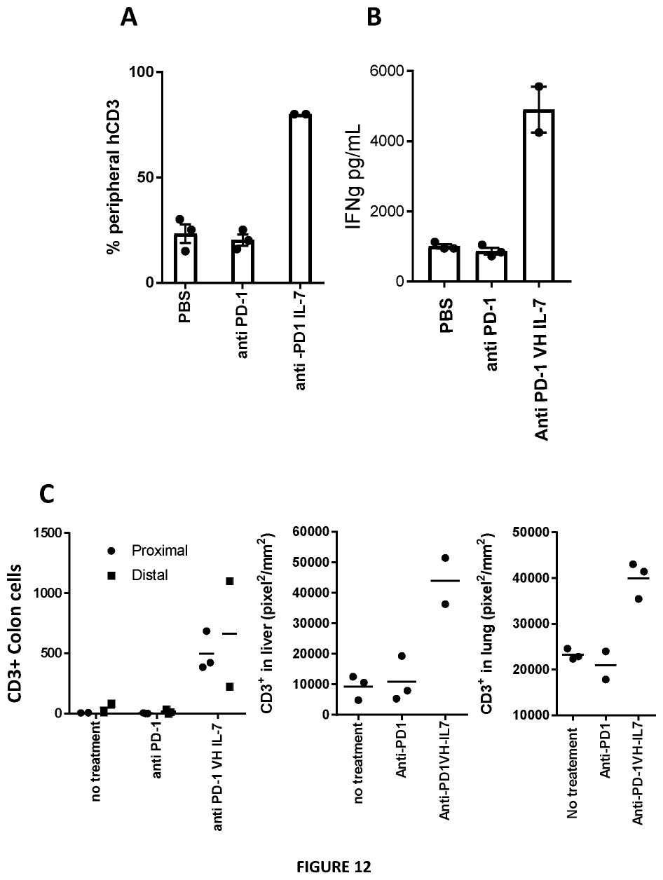

In a humanized mouse model (FIG. 12), inventors observed an increase in the percentage of CD3 positive cells in peripheral blood and an increase of IFNg secretion after treatment with Bicki anti-PD1-IL7 molecules compared to anti-PD1 antibody and negative control with PBS, confirming in-vivo that Bicki molecule increases human T-cell expansion, survival and activation This difference is furthermore correlated with high T cell infiltration into the colon but also in the liver and lungs confirming that IL-7 part of the Bicki molecules promotes human T cell migration in inflamed tissues.

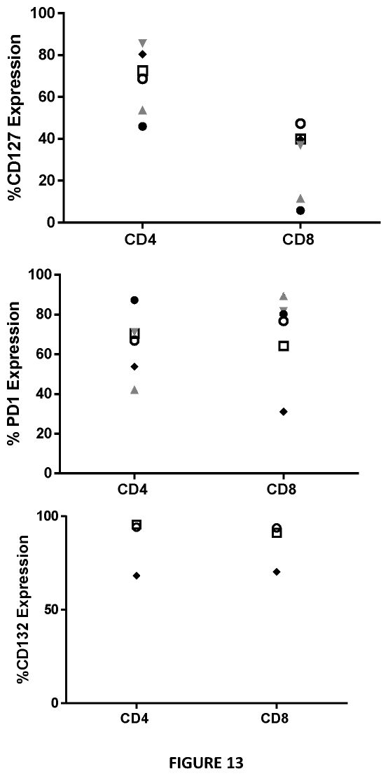

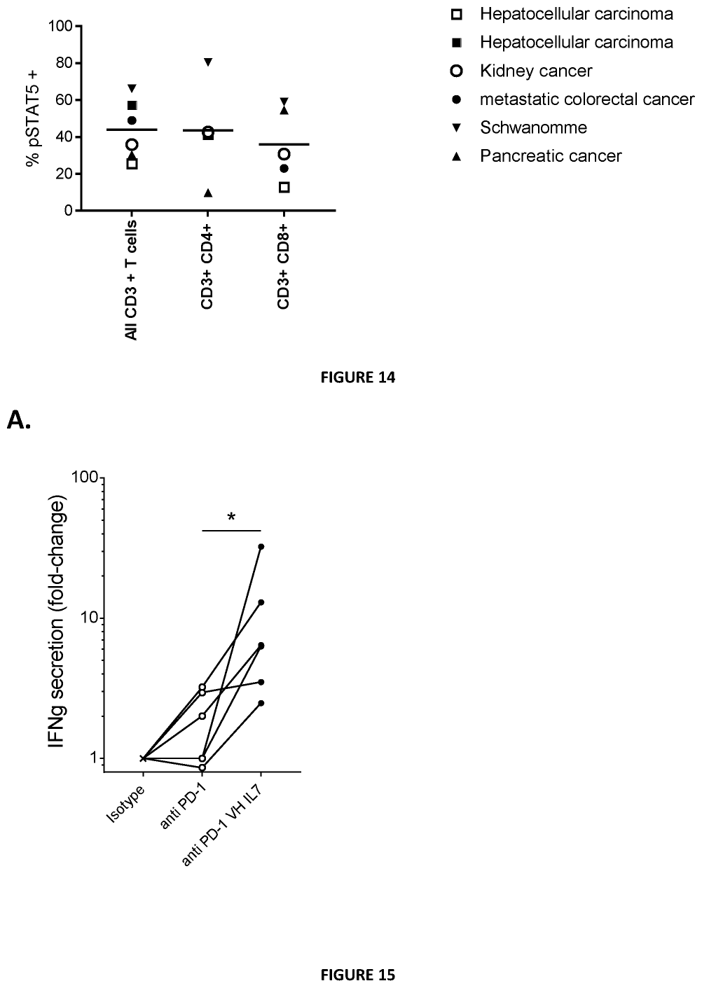

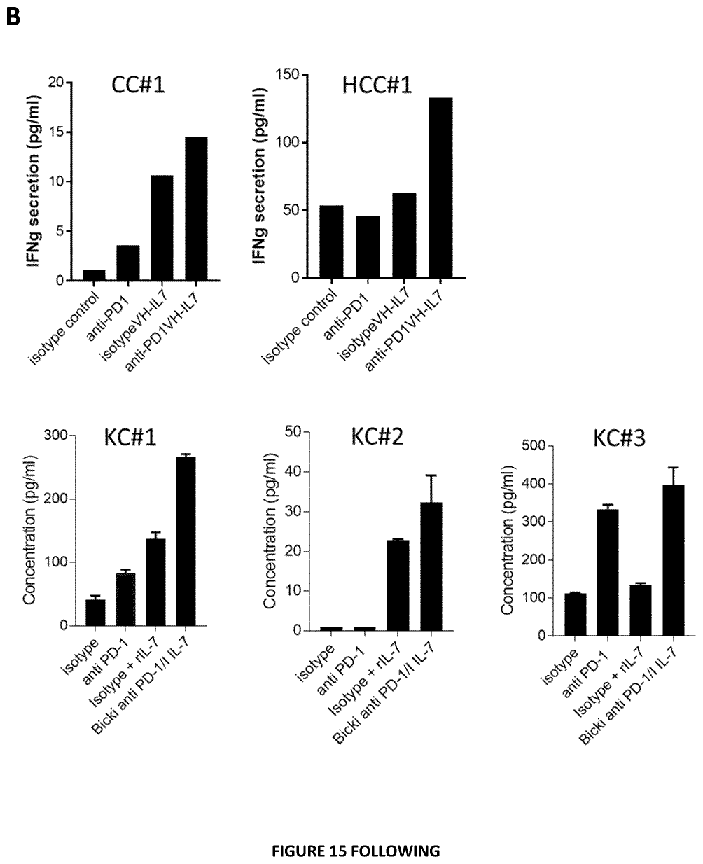

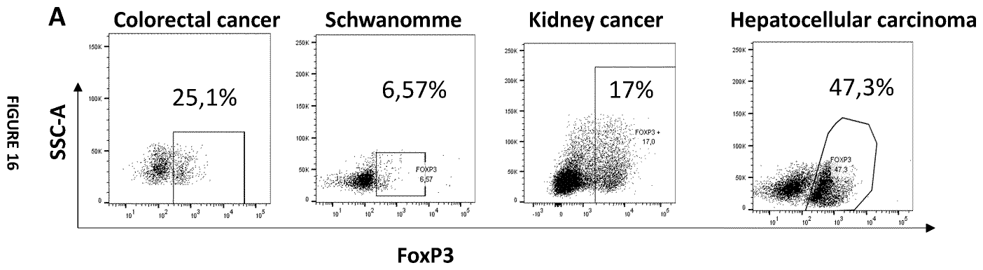

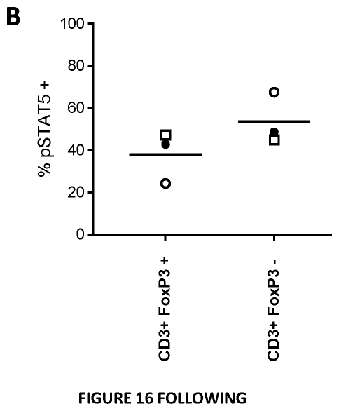

The T cell phenotype analysis into human tumors demonstrates that intratumoral T cells express PD-1, IL-7R/gamma common chain (CD127 and CD132) comforting that Bicki anti-PD1-IL7 molecules might be efficient locally to stimulate intratumoral human T cells in contrast to what was predicted from the literature (FIG. 13). Indeed, intratumoral T cells respond to IL-7 and Bicki anti-PD1-IL7 molecules as shown by pSTAT5 staining (FIG. 14) confirming ex-vivo on human T-cells purified from tumors that Bicki anti-PD1-IL7 molecules can be beneficial for treatment of various tumor subtypes. Then using an ex-vivo culture model of human tumor explants, containing tumors cells but also all the tumor micro-environment including human immune cells and stromal cells, treated with anti-PD1, isotypeVH-IL7, Bicki anti-PD1-IL7 molecules or IL-7 (FIG. 15). The IFNg secretion was analyzed in the supernatant as a specific marker of T-cell activation and a surrogate marker associated with the response to anti-PD1 in various clinical trials. Inventors observed that bicki anti PD-1 IL7 treated human tumor cultures significantly secrete more IFNg confirming that IL7 acts locally in tumor bed and increases locally T-cell activation. Interestingly, human tumor cell cultures treated with Bicki anti-PD1-IL7 molecules secreted more IFNg than anti-PD1 alone or IL7/isotype VHIL7alone, demonstrating a synergistic effect of combining anti-PD-1 with IL-7 to activate intratumoral T cells (FIG. 15B). The percentage of intratumoral T reg (FoxP3+) was also analyzed from cancer patients (colorectal cancer, schwannoma, kidney cancer and hepatocellular carcinoma). FIG. 16 A presents the results indicating that each cancer tested presents Treg cells that could be a target for the Bicki anti-PD1-IL7 molecules to disarm Treg cells. Then, the STATS activation (pSTAT5) in intratumoral effector (FoxP3-) and regulator T cells (FoxP3+) was analyzed after treatment with Bicki anti-PD1-IL7 molecules. Results obtained with cells coming from colorectal cancer, schwanomma and pancreatic cancer and presented FIG. 16B show that Bicki molecules are able to activate IL-7R pathway in intratumoral effector and regulator T cells. This result underlines the double edge blade of the Bicki anti-PD1-IL7 molecules to favor effector T cells activation into the tumor while abrogating Treg suppressive activity.

### Example 9. Mutations of Fc Fused IL-7 Modify Binding to IL-7R and pSTAT5 Signaling and Improves Pharmacokinetics In Vivo

To obtain IL-7 mutants, amino-acids implicated in the interaction IL7 to CD127 were substituted with amino-acid possessing similar nature and properties. Several mutants were generated, namely Q11E, Y12F, M17L, Q22E, D74E, D74Q, D74N, K81R, W142H, W142F and W142Y.

IL-7 disulfide bonds were disrupted by replacing cysteine residues by serine residues, leading to the substitution C2S-C141S+C34S-C129S (mutant named “SS1”), or C2S-C141S+C47S-C92S (mutant named “SS2”), or C47S-C92S+C34S-C129S (mutant named “SS3”).

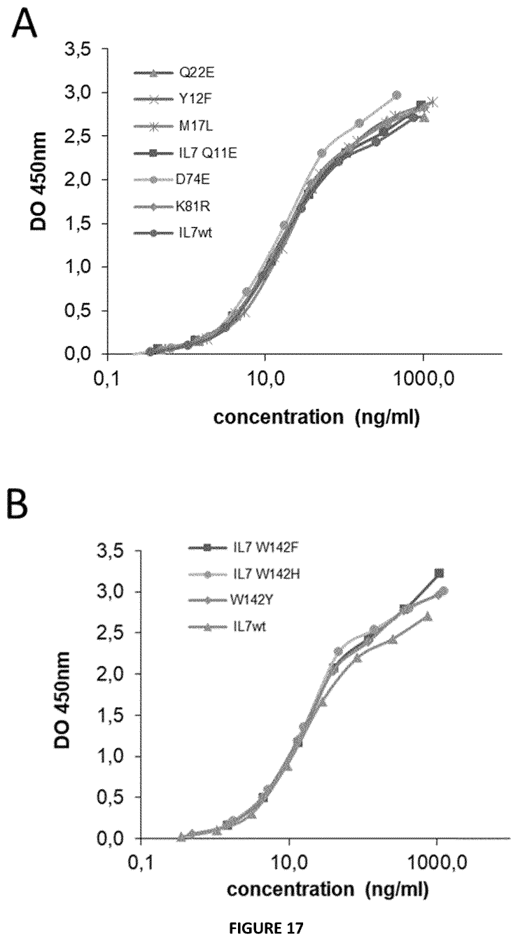

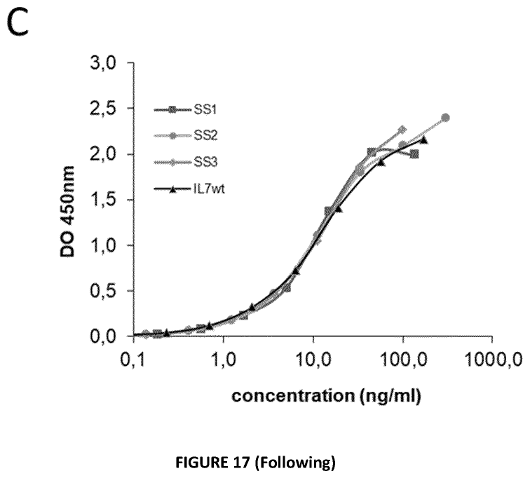

TABLE 3
ED50 determination from FIGS. 17A, B and C refers to the concentration
required to reach 50% of the binding to CD127 receptor. Each table
represent a different experiment and can be compared to the positive
control IgG4 G4S3 IL7WT.

| Samples | EC50 ng/mL |
|---|---|
| IgG4 G4S3 IL7 WT | 18.4 |
| IgG4 G4S3 IL7 Q11E | 18.49 |
| IgG4 G4S3 IL7 Y12F | 22.27 |
| IGG4 G4S3 IL7 M17L | 20.96 |
| IGG4 G4S3 IL7 Q22E | 17.44 |
| IgG4 G4S3 IL7 D74E | 103.94 |
| IgG4 G4S3 IL7 K81R | 20.18 |
| IgG4 IL7 G4S3 W142F | 34.86 |
| IgG4 G4S3 IL7 W142H | 136.32 |
| IGG4 G4S3 IL7 W142Y | 44.6 |

TABLE 4
Binding of WT versus mutated IL-7 to CD127 receptor.

| Samples | Ka (1/Ms) | Kd2 (1/s) | KD (M) |
|---|---|---|---|
| IgG4 Fc G4S3 IL-7 WT | 5.76E+06 | 1.22E−04 | 4.14E−11 |
| IgG4 Fc G4S3 IL-7 W142H | 5.02E+05 | 2.56E−03 | 5.68E−08 |
| IgG4 Fc G4S3 Fc IL-7 SS2 | 6.11E+05 | 1.55E−03 | 7.22E−09 |
| IgG4 Fc G4S3 Fc IL-7 SS3 | 1962 | 6.02E−4 | l.36E−6 |

Affinity assessment by Biacore of fused anti PD-1 IL-7 for CD127. A two-state reaction model was used for analysis.

TABLE 5
Binding of WT versus mutated IL-7 to CD132 receptor.

| Samples | KD CD132 |
|---|---|
| IgG4 alone | 2.50E−06 |
| IgG4 G4S3 IL-7 WT | 1.18E−07 |
| IgG4 Fc G4S3 IL-7 W142H | 5.72E−07 |
| IgG4 Fc G4S3 Fc IL-7 SS2 | 3.10E−06 |

Affinity assessment by Biacore of the complex CD127 + IgG fused IL-7 on CD132. A steady-state reaction model was used for analysis.

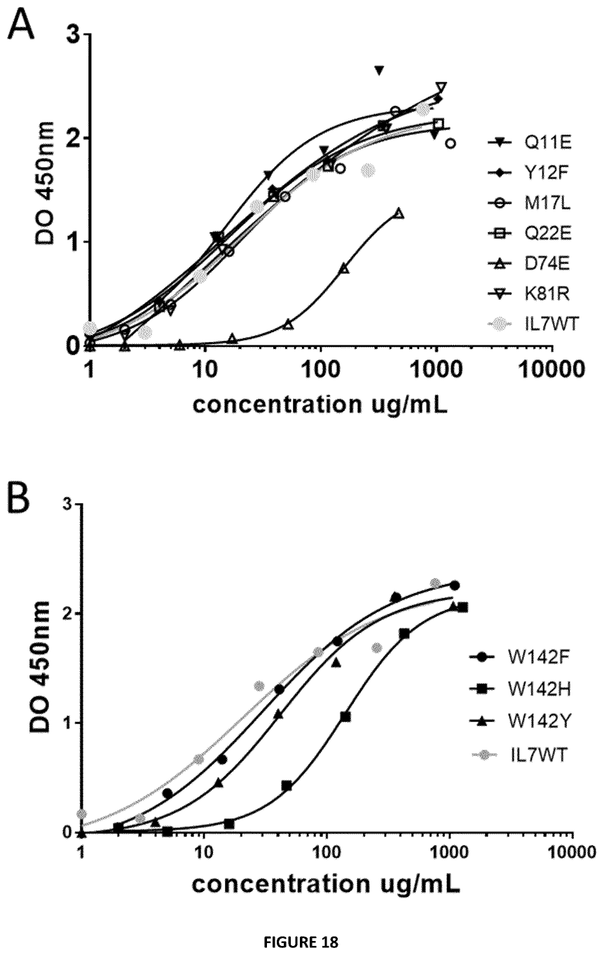

TABLE 6
ED50 determination from FIGS. 18A, B and C refers to the concentration
required to reach 50% of the pSTAT5 signal in this assay for each anti
PD-1 IL-7 molecule. Each table represents a different experiment with a
different donor and each table can be compared to the positive control
IgG4 G4S3 IL7WT.

| Samples | EC50 ng/mL |
|---|---|
| IgG4 G4S3 IL7 WT | 76 |
| IgG4 IL7 Q11E | 77 |
| IgG4 G4S3 IL7 Y12F | 66 |
| IGG4 G4S3 IL7 M17L | 128 |
| IGG4 G4S3 IL7 Q22E | 84 |
| IgG4 G4S3 IL7D74E | 389 |
| IgG4 G4S3 IL7 K81R | 79 |
| IgG4 G4S3 IL7 W142F | 102 |
| IgG4 G4S3 IL7 W142H | 861 |
| IgG4 G4S3 IL7 W142Y | 208 |
| IgG4 G4S3 IL7 WT | 0.52 |
| IgG4 G4S3 IL7 SS2 | 2401 |
| IGG4 G4S3 IL7 SS3 | 4348 |

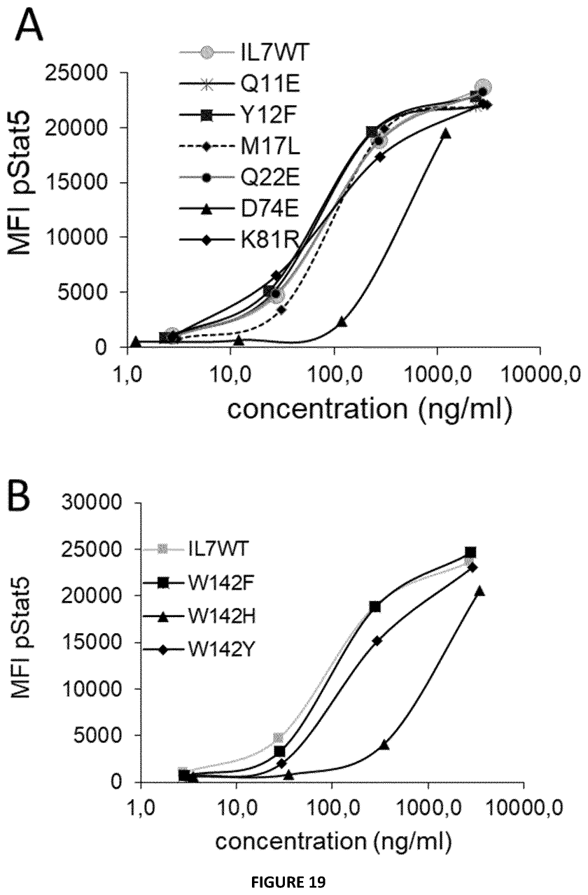

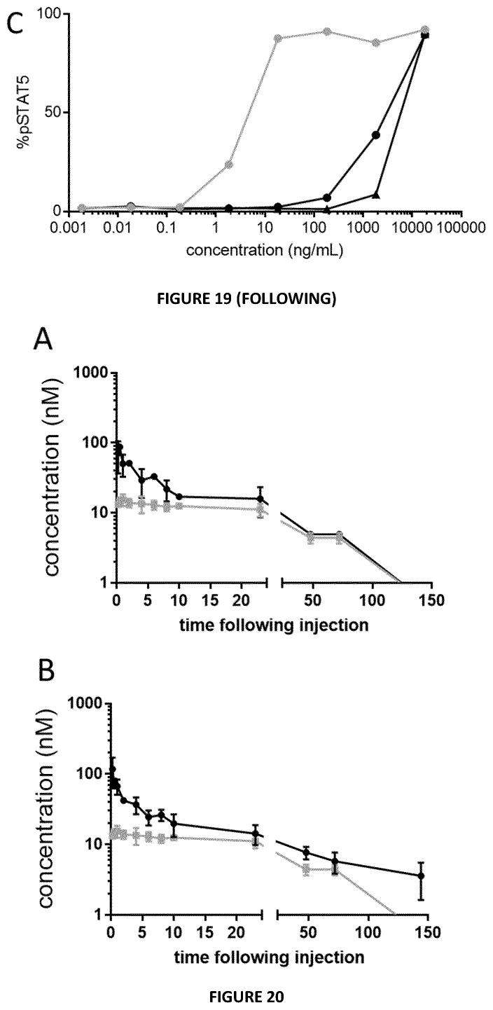

TABLE 7
Cmax, area under the curve and half-life determination from FIG. 19.
Cmax was calculated at the time point 15 minutes following anti PD-1
IL7 injection. AUC was calculated from 0 to 144 hours following
injection of the anti PD-1 IL-7.

| | C max | Area under curve |
|---|---|---|
| Samples | obtained (nM) | (AUC) |
| IgG4 G4S3IL7WT | 13.22 | 121.4 |
| IgG4 G4S3 IL7D74E | 89.19 | 151.9 |
| IgG4 G4S3IL7 W142F | 98 | Undetermined |
| IgG4 G4S3IL7 W142H | 141 | 248.2 |
| IgG4 G4S3IL7 W142Y | 70 | Undetermined |
| IgG4 G4S3 SS2 | 69.9 | 361.6 |
| IgG4 G4S3 SS3 | 140.6 | 466.5 |

The substitution of one amino-acid in IL7 sequence did not modify its capacity to bind PD-1 receptor (FIGS. 17A, B and C). However, these mutations modify its biological activity as shown by CD127 binding and pSTAT5 signaling in ex vivo T cells assay (FIGS. 18 and 19 and Tables 3 and 6). The mutation D74E and W142H are the most efficient mutation to decrease both IL-7 binding to CD127 and activation of pStat5 in T lymphocytes (FIGS. 18A, 18B and 19A, 19B and Tables 3 and 7). In another experiment, the effect of disulfilde bounds disruption was analyzed (FIG. 18C). At high concentration (10 μg/ml), SS2 or SS3 were able to activate pStat5 in T lymphocyte, with 3 log deviation from IL-7 WT (FIG. 18C and Table 6). To confirm the binding capacity of those mutants, a Biacore assay was performed to determine the KD (equilibrium dissociation constant between the receptor and its antigen, see Table 4). Mutants SS2 and W142H have a lower affinity to CD127 with a KD close to 7 to 57 nM. The SS3 mutant has the lowest affinity for the CD127 with a KD close to 3 μM. The affinity for the CD132 receptor was also assessed as shown on Table 5. In this experiment, IgG4 alone was used as baseline KD affinity as CD127 dimerizes with CD132 in the absence of IL-7. IL-7 mutant W142H binds to CD132 but with 5-fold higher affinity compared to the IgG IL-7WT. This data demonstrates that the mutation W142H decreases binding to CD127 and redirect binding of IL-7 toward the CD132 receptor, leading to a loss of pSTAT5 activation in T cells as shown on FIG. 18. In contrast, the inventors observed in the condition tested that SS2 mutant loses the capacity to bind to CD132 receptor, suggesting that the SS2 mutant preferentially binds to CD127 over CD132 receptor, leading to a decrease pSTAT5 activity in T cells (FIG. 19).

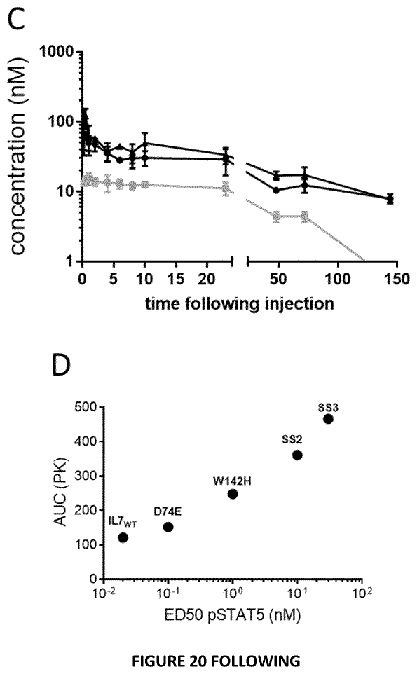

To determine pharmacokinetics/pharmacodynamics of the anti PD-1 IL-7 in vivo, mice were intravenously injected with one dose of IgG-IL-7 (34,4 nM/kg). Plasma drug concentration was analyzed by ELISA specific for human IgG. FIG. 19 and Table 7 show that IgG4 IL-7 WT molecules have rapid distribution as the Cmax (maximal concentration 15 minutes following injection) obtained is 30-fold lower than theorical concentration. All the W142Y, F, H mutants tested depicted a better distribution profile with a Cmax 5 to 10-fold higher than the IL-7 WT (FIG. 19A and Table 7). The W142H mutant presents the best Cmax. Anti PD-1 IL-7 D74E mutant also demonstrated a good Cmax. The mutants SS2 and SS3 exhibit the best PK profile with a 7 to 13-fold higher Cmax than IL-7 WT and good linear profile curve. In parallel, the AUC (Area under the curve) was determined (Table 7 and FIG. 20D), the AUC gives insight into the extent of drug exposure and its clearance rate from the body. These data demonstrate that the AUC increased with the IL-7 mutants meaning that the IL-7 mutants have an improved drug exposure. As represented in FIG. 20D, the inventors observed that the drug exposure correlates with the IL-7 potency of the mutant (measured by pSTAT5 EC50). In conclusion, the affinity of IL-7 is correlated with the pharmacokinetics of the product. Decreasing affinity of IL-7 to their receptors CD127 and CD132 improves the absorption and distribution of the IL_7 bifunctional molecules in vivo.

### Example 10: The Addition of a Cysteine at the C-Terminal Domain at the C-Terminal Domain of the IgG Decreases the Flexibility of the IL7 Molecule and Improve Pharmacokinetics In Vivo

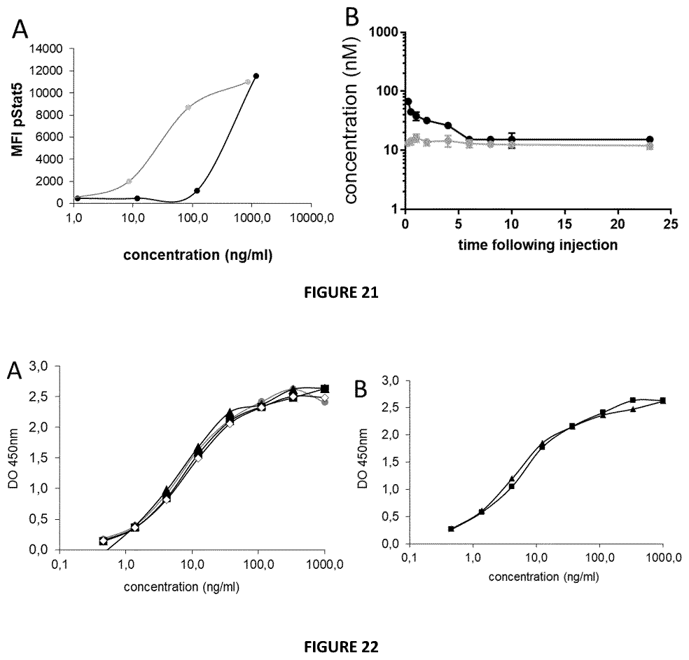

The addition of a cysteine at the C-terminal domain at the C-terminal domain of the IgG was also tested to create an additional disulfide bond and potentially restrict the flexibility of the IL-7 molecule. This mutant was named “C-IL-7”. FIG. 21 shows that the addition of a disulfide bounds in the IgG structure decreases pSTAT5 activity of the IL-7 compared to the anti PD-1 IL7 WT bifunctional molecule (FIG. 21A) and increases Cmax (5-fold) in the pharmacokinetics assay in vivo (FIG. 21B).

### Example 11: Anti PD-1 IL-7 Mutants Constructed with an IgG1N298A Isotype has a Better Binding to IL-7R, a Higher pSTAT5 Signaling and a Good Pharmacokinetics Profile In Vivo

Different isotypes of the anti PD-1 IL-7 bifunctional molecules were tested with IgG4m (S228P) or IgG1m (N298A or N297A depending on the numbering method). IgG4 isotype comprises the S228P mutation to prevent Fab arm-exchange in vivo and the IgG1 isotype comprises the N298A mutation that abrogates IgG1 isotype binding to FcγR receptors that may reduce the non-specific binding of the immunocytokine (mutant named “IgG4m” or “IgG1N298A”). Then, Anti PD-1 IL-7 bifunctional molecule was constructed with 2 different isotypes, IgG1 mutated in N297A (called IgG1m) isotype versus the IgG4 S288P isotype (called IgG4m) to determine whether the isotype structure modify the biological activity of IL-7 and its pharmacokinetics profile.

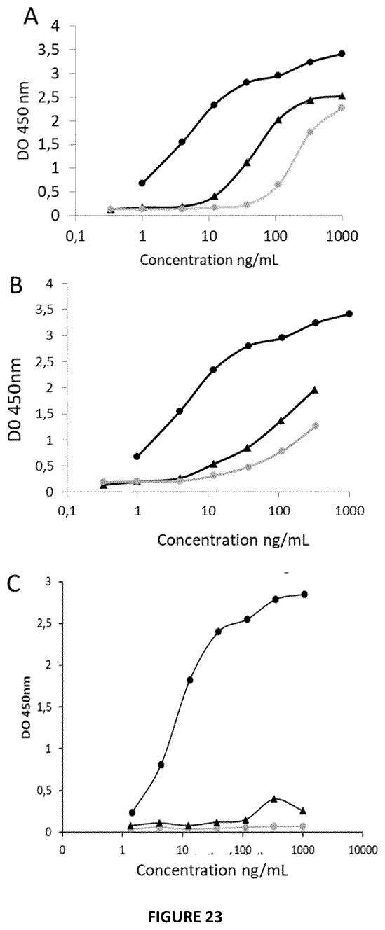

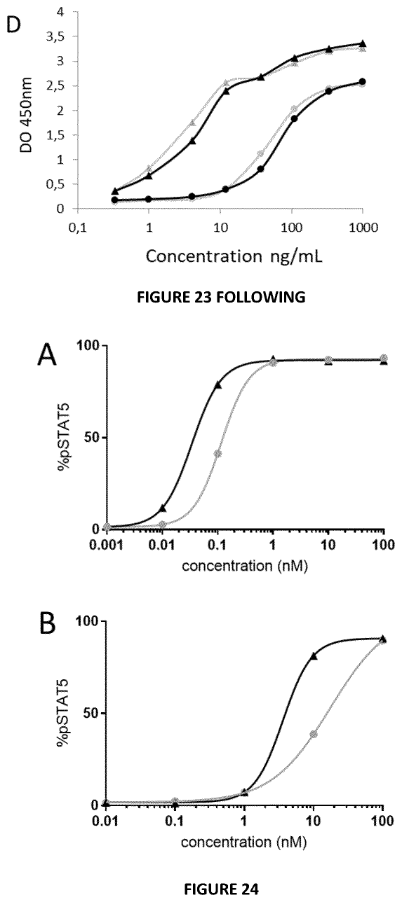

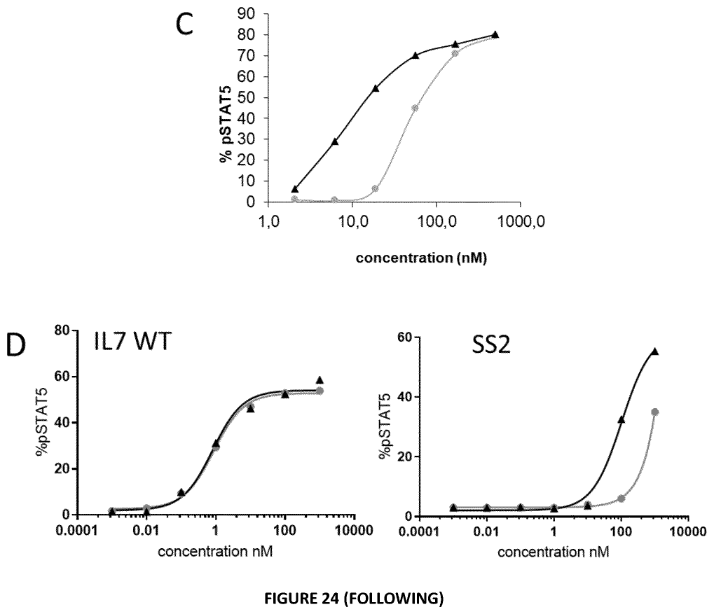

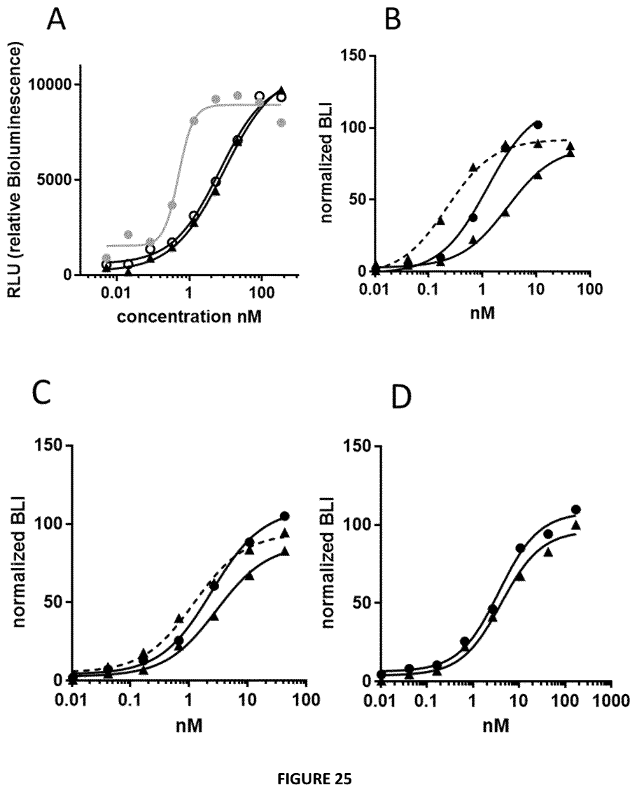

FIGS. 22A and 22B demonstrate that the anti PD-1 IL7 bifunctional molecules constructed with the IgG4m or IgG1m isotype have the same binding properties to PD-1 receptor, showing that the isotype does not modify the conformation of the VH and VL and the affinity of the anti PD-1 antibody for PD-1. However, the inventors observed that the IgG1m isotype unexpectedly improves the binding of the IL-7 D74, SS2 and slightly SS3 on CD127 (FIGS. 23A, B, C and D) and pSTAT5 activation on human PBMCs (FIGS. 24A, B and C). This increase in pSTAT5 signalling was confirmed for the SS2 mutant on another T cell line (Jurkat cells expressing PD-1 and CD127, see FIG. 24D), but in a surprising manner, the IgG1m isotype does not modify pSTAT5 activity of the anti PD-1 IL-7 WT bifunctional molecule, suggesting that the IgG1m isotype only improves the activity of the IL-7 mutants. To determine the capacity of bifunctional molecule comprising an anti-PD1 antibody and an IL7 mutant to reactivate TCR mediated signaling, a NFAT Bioassay was performed. Results presented FIG. 25A show that the bifunctional molecule is better than an anti-PD1 or an anti-PD1+rIL7 (as separate compounds) to activate TCR mediated signaling (NFAT), demonstrating a synergistic effect of the bifunctional molecule on PD1+ T cells. The inventors next assessed the synergistic capacity of the bifunctional molecule comprising an anti PD-1 antibody and an IL-7 mutant (with mutation D74E, W142H or SS2) constructed with an IgG4m versus IgG1m isotype (FIGS. 25B, C, D). All the mutants tested conserve a synergistic effect on activating NFAT signaling with a level of activation correlated with their capacity to activate pSTAT5 signaling, in particular for bifunctional molecule with IL-7 D74E with IgG4m.

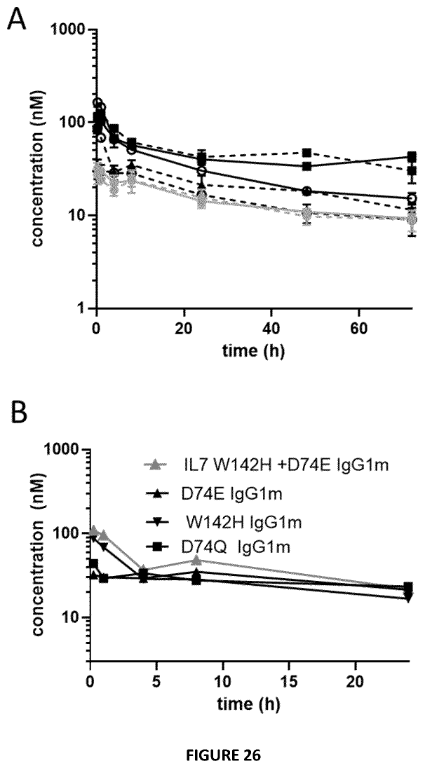

Pharmacokinetics study in mice demonstrate that IgG1 isotype does not modify the drug exposure for the IL7WT and SS3 molecule and a minimal impact on W142H molecule (FIG. 26A). Altogether these data show that an optimized isotype (IgG1m) is sufficient to enhance biological activity of the mutants while conserving a good pharmacokinetics of the product in vivo. With the IgG1m isotype, other IL-7 mutants were tested: D74N, D74Q and combination of D74E+W142H mutation. No differences with the anti PD-1 IL-7 D74E mutant were observed on pSTAT5 activation (FIG. 25B) and pharmacokinetics (FIG. 26B).

The double mutant D74E+W142H displayed similar profile compared to W124H IgG1 and the D74Q displayed a similar profile compared to D74E mutant. The inventors also constructed bifunctional molecules with IgG1m isotype+YTE mutation (M252Y/S254T/T256E). This mutation has been described to increase half life of antibody by increasing the binding to FcRn receptors. As shown on FIG. 23D, the YTE mutation does not modify the pSTAT5 signaling of the bifunctional molecule comprising the D74 or the W142H mutant.

### Example 12: The Mutation K444A into the C-Terminal Lysine Residue Does Not Affect Pharmacokinetics In Vivo

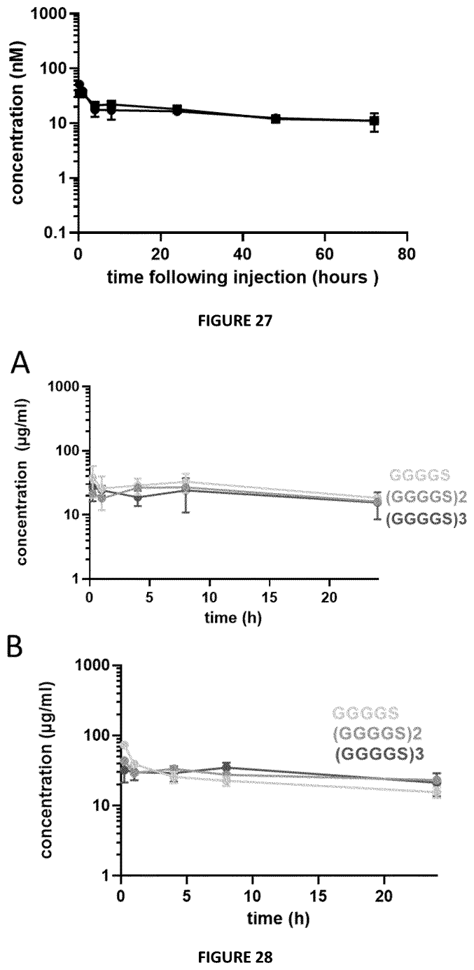

All subclass of Human IgG carries a C-terminal lysine residue of the antibody heavy chain (K444) that can be cleaved off in circulation. This cleavage in the blood may potentially compromises the bioactivity of the Immunocytokine by releasing the linked IL-7 to IgG. To circumvent this issue, K444 amino acid in the IgG domain was substituted by an alanine to reduce proteolytic cleavage, a mutation commonly used for antibodies. As shown in the FIG. 27, similar curve was obtained between IgG WT IL-7 versus IgG K444A IL-7 suggesting that the mutation does not affect the pharmacokinetic profile of the drug.

### Example 13: Linker Between IgG Antibody Does Not Modify Pharmacokinetics In Vivo but Improves Activation of pSTAT5 Signaling

Different linkers between IgG Fc domain and IL-7m were tested to modify flexibility. Several conditions were tested (e.g. no-linker, GGGGS, GGGGSGGGS, GGGGSGGGGS, GGGGSGGGGSGGGGS). For the example 1 and 2, a linker (G4S)3 between the C-terminal domain of the Fc and the N-terminal domain of the IL-7 was used for the IgG4m-IL7 and IgG1m-IL-7 constructions, respectively. This linker allowed high flexibility and improvement of IL7 activation signal. To reduce affinity of IL7 to CD127 and improve the pharmacokinetics, different constructions were tested with varying the length of the linker (no linker, G4S, (G4S)2 or (G4S)3). For comparison, IgG1m or IgG4m Fc IL-7 WT was also generated with various linkers.

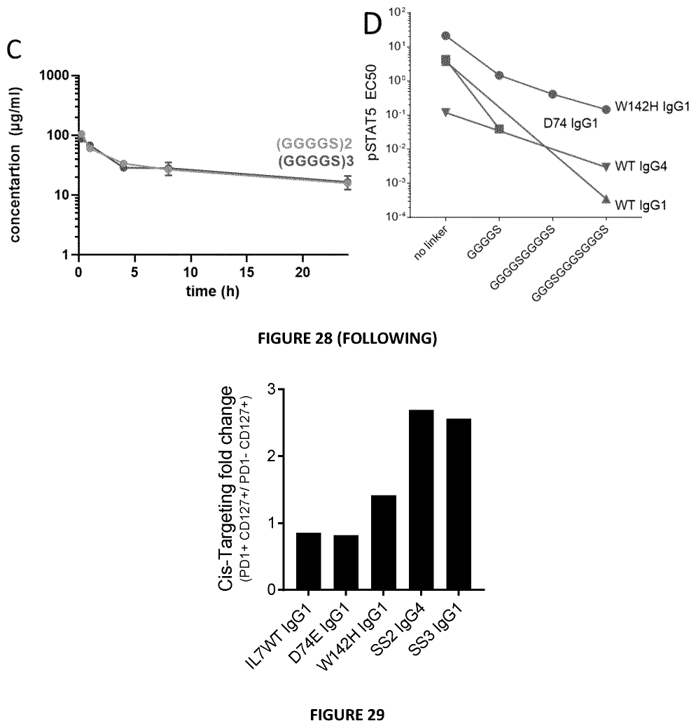

Pharmacokinetics study demonstrate that the length of the linker has no impact on the distribution, absorption and elimination of the product for the construction tested: Anti PD-1 IL7 WT (FIG. 28A), anti PD-1 IL-7 D74 (FIG. 28B) and anti PD-1 IL-7 W142H (FIG. 28C). However, the length of linker influences the activation of pStat5 as shown in FIG. 28D. Indeed, Anti PD-1 IL7 constructed with a linker (G4S)3 are more potent in activating pSTAT5 signaling compared to anti PD-1 IL-7 constructed with (G4S)2 or G4S3 linker and even more potent compared to anti PD-1 IL-7 constructed without linker. These data underscore the use of a (G4S)3 linker to allow flexibility of the IL-7 without compromising the pharmacokinetics of the drug in vivo.

### Example 14: The Anti PD-1 IL-7 Mutants Allow Preferential Binding on PD-1+CD127+ Cells Over PD-1-CD127+ Cells

Next, the inventors assessed the capacity of the anti PD-1 IL-7 bifunctional molecule to target PD-1+ T cells. Jurkat cells expressing CD127+ or co-expressing CD127+ and PD-1+ were stained with 45 nM of the following bifunctional molecules: anti PD-1 IL-7 WT, D74, W142H, SS2 and SS3. The binding was detected with an anti IgG-PE (Biolegend, clone HP6017) and analyzed by flow cytometry.

Results: FIG. 29 shows that anti PD-1 IL-7 WT and D74 mutant bind with similar efficacy to PD-1+/CD127+ cells versus PD-1-/CD127+ cells, whereas anti PD-1 IL-7 mutant SS2, SS3 binds with 2 to 3-fold higher efficacy to PD-1+/CD127+ cells versus PD-1-/CD127+ cells. The anti PD-1 IL-7 W142H bifunctional molecule shows an intermediate effect and binds with 1,4-fold higher efficacy to PD-1+/CD127+ cells. Altogether, these data show that the 11-7 mutation not only allows a better pharmacokinetics of the drug, but also allows the preferential binding of IL-7 on PD-1+ cells, i.e targeting of the drug on the same cell. This aspect has an interest for the biological activity of the drug in vivo, as the anti PD-1 IL-7 will concentrate the IL-7 on PD-1+CD127+ exhausted T cells into the tumor microenvironment over CD127+ naïve T cells.

### Example 15: the Molecule Bicki Anti PD-1 IL-7 Allows Proliferation of CD4 and CD8 T Cells and Demonstrates Preclinical Safety In Vivo in Cynomolgus Monkeys

Cynomolgus monkeys were injected with 6,87 nM/kg (n=2) or 34,35 nM (n=1) (equivalent to 1 mg/kg or 5 mg/Kg) for an antibody. Blood analysis was performed until Day 15 or 4 hours following injection. Proliferation of CD4/CD8 T cells or B cells was assessed by flow cytometry in the blood using Ki67 marker. pSTAT5 was analyzed at multiple time points in CD3+ T cells by FACS after fixation and permeabilization of cells.

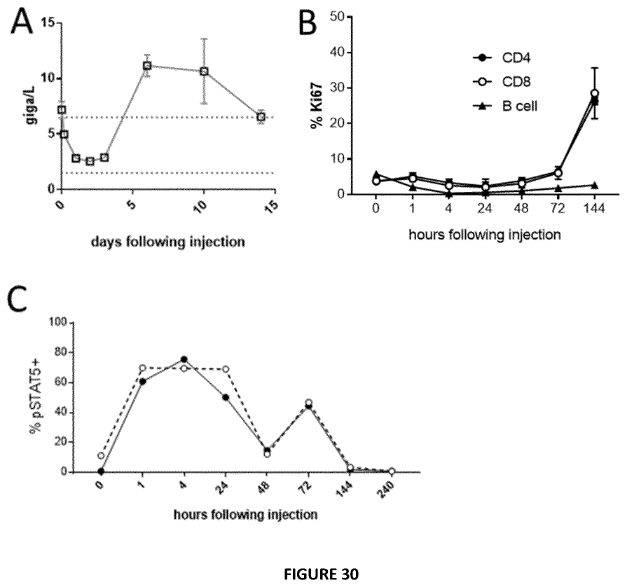

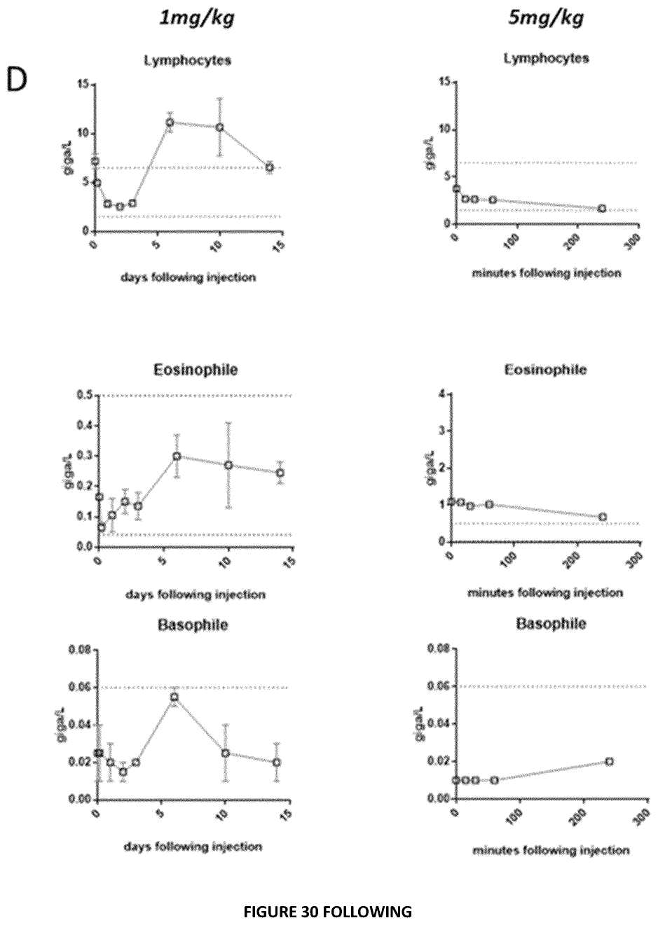

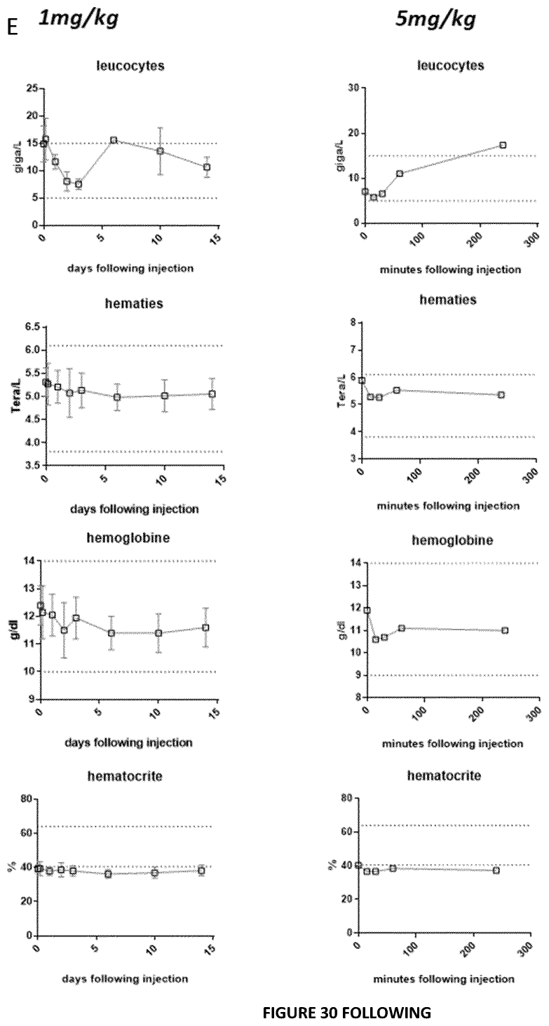

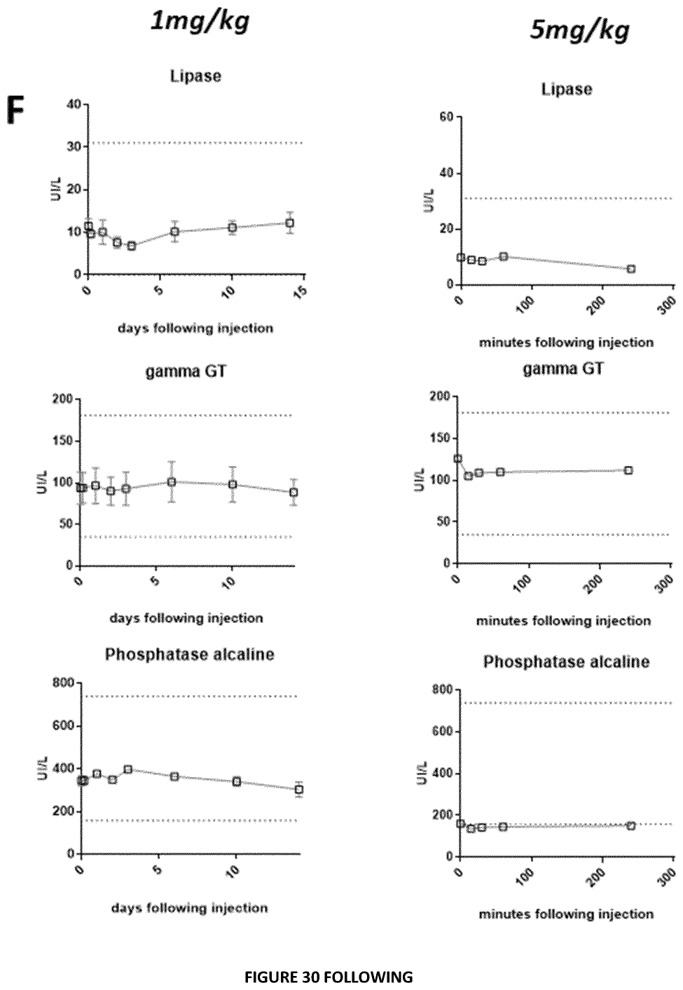

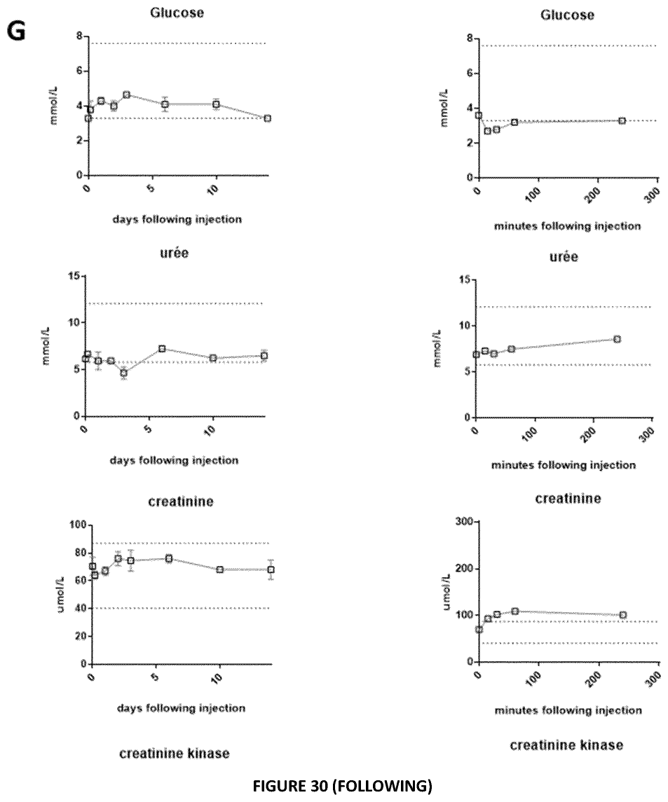

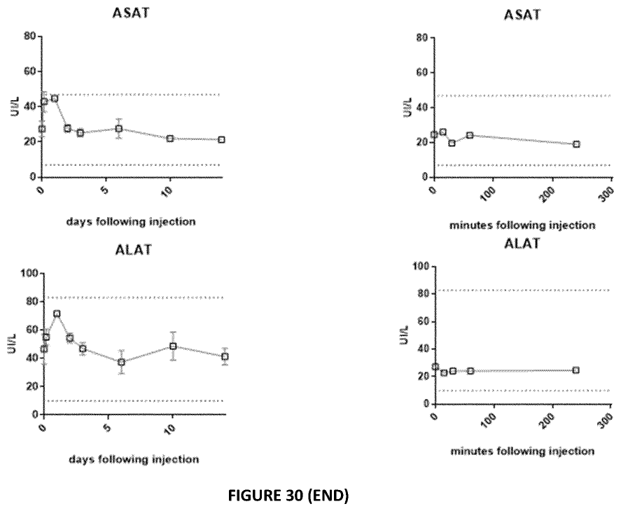

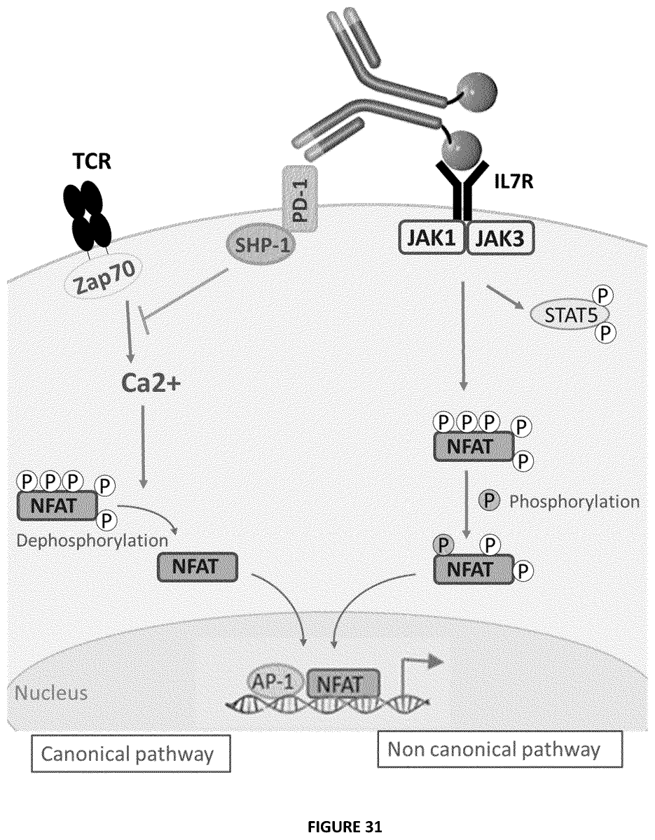

Results: FIGS. 30A and B shows that a single injection of the bicki anti PD-1 IL-7 induces proliferation of CD4 and CD8 T cells but does not induce proliferation of B cells. After injection, a rapid activation of pSTAT5 was observed with a maximum activation from 1 to 24 hours as demonstrated on FIG. 30C. The drug was well tolerated ad safe at this dose, as shown on FIGS. 30 D/E/F clinical parameters (biochemical and blood cell count) were in or close to the normal range after injection of the molecule. These data show that the molecule is able to activate T cells in vivo and show preclinical safety.

Conclusion

Bicki anti-PD1-IL7 antibody format with an IL-7 cytokine fused to the C-terminal domain of the Fc portion maintains the binding to CD127 receptor whereas unexpectedly, IL7 fused to the N-Terminal domain of an Fc (IL7-Fc) loses its binding capacity. Bicki format will be more potent in term of IL-7R activation and elimination half-life in patient. Furthermore, in comparison to IL7-Fc compound, Bicki anti-PD1-IL7 antibody format allows accumulation of IL-7 in PD-1+ T cells infiltrates and re-localization of IL-7 on PD-1+ T cells with increased T-cell activation using Bicki as compared to the combination of the use of an anti-PD1+ an IL7 recombinant protein. Furthermore, Bicki anti-PD1-IL7 molecule is a single double edge sword, on one hand increasing proliferation of effector T cells and their activation reflected by the secretion of IFNg cytokine both in vitro and in vivo model, and on the other hand by disarming Treg suppressive function on T-cells. This has been demonstrated on human T cells isolated from various tumor type and indications suggesting that Bicki anti-PD1-IL7 molecule could be beneficial for various tumor subtypes. Tumors resistant to PD-1 therapy present T cell exclusion. It's known that the PD-1 response is correlated with quantity of tumor infiltrating T cells. Bicki anti-PD1-IL7 molecules increase integrins expression at cell surface suggesting that the bifunctional molecules of the invention promote T-cell infiltration into the tumor in multiple cancer subtypes.

Materuak and Method

ELISA Binding PD1

For activity ELISA assay, recombinant hPD1 (Sino Biologicals, Beijing, China; reference 10377-H08H) was immobilized on plastic at 0.5 μg/ml in carbonate buffer (pH9.2) and purified antibody were added to measure binding. After incubation and washing, peroxidase-labeled donkey anti-human IgG (Jackson Immunoresearch; USA; reference 709-035-149) was added and revealed by conventional methods.

Affinity Measurement Using Biacore Method

Affinity assessment by Biacore of IgG fused to IL-7 on its heavy chains for CD127 (A) or CD132 (B). CD127 (Sinobiological, 10975-H03H-50) was immobilized onto a CM5 biochip at 20 μg/ml and the indicated protein were added at serial concentrations (0.35; 1.1; 3.3; 10; 30 nM). Affinity was analyzed using two state reaction models. To assess affinity of IL-7 to CD132, CD127 was immobilized on the CM5biochip and each IL-7 construction was injected at a concentration of 30 nM. The CD132 receptor (Sinobiological 10555-H08B) was added at different concentrations, e.g. 31.25, 52.5, 125, 250, 500 nM. A steady state affinity model was used for analysis.

CD127 Binding ELISA

CD127 binding was assessed by a sandwich ELISA method. Recombinant proteins targeted by the antibody backbone were immobilized, then antibodies fused IL-7 preincubated with CD127 recombinant protein (Histidine tagged, Sino ref 10975-H08H) were incubated. Revelation was performed with a mixture of an anti-histidine antibody (MBL #D291-6) coupled to biotin and streptavidin coupled to Peroxidase (JI 016-030-084). Colorimetry was determined at 450 nm using TMB substrate.

pSTAT5 Analysis In Vivo

PBMCs isolated from peripheral blood of human healthy volunteers were incubated 15 minutes with recombinant IL-7, or IgG fused IL-7. Cells were then fixed, permeabilized and stained with an AF647 labeled anti-pSTAT5 (clone 47/Stat5(pY694)). Data were obtained by calculating MFI pSTAT5 in CD3+ T cell population.

Pharmacokinetics of the IgG Fused IL-7 In Vivo

To analyze the pharmacokinetics of the IL-7 immunocytokine, a single dose of the molecule was intra-orbitally injected to BalbcRJ mice (female 6-9 weeks). Drug concentration in the plasma was determined by ELISA using an immobilized anti-human light chain antibody (clone NaM76-5F3) diluted serum containing IgG fused IL-7. Detection was performed with a peroxidase-labeled donkey anti-human IgG (Jackson Immunoresearch; USA; reference 709-035-149) and revealed by conventional methods. Cynomolgus monkeys were intravenously injected with Bicki IL-7 at 2 doses. Blood was harvested at multiple time point following injection (15/30 min, 1/2/4 hours, 1/2/3/6/10/14 days) to analyze biochemical and cell count.

T Cell Activation Assay Using Promega Cell-Based Bioassay

The capacity of anti-PD-1 antibodies restore T cell activation was tested using Promega PD-1/PD-L1 kit (Reference J1250). Two cell lines are used (1) Effector T cells (Jurkat stably expressing PD-1, NFAT-induced luciferase) and (2) activating target cells (CHO K1 cells stably expressing PDL1 and surface protein designed to stimulate cognate TCRs in an antigen-independent manner. When cells are cocultured, PD-L1 /PD-1 interaction inhibits TCR mediated activation thereby blocking NFAT activation and luciferase activity. The addition of an anti-PD-1 antibody blocks the PD-1 mediated inhibitory signal leading to NFAT activation and luciferase synthesis and emission of bioluminescence signal. Experiment was performed as per as manufacturer recommendations. Serial dilutions of the PD-1 antibody were tested. Four hours following coculture of PD-L1+ target cells, PD-1 effector cells and anti PD-1 antibodies, BIOGLO luciferin substrate was added to the wells and plates were read using TECAN luminometer.

Antibodies and Bifunctional Molecules

The following antibodies and bifunctional molecules have been used in the different experiments disclosed herein: Pembrolizumab (Keytrudra, Merck) Nivolumab (Opdivo, Bristol-Myers Squibb) , and the bifunctional molecules as disclosed herein comprising an anti-PD1 humanized antibody comprising a heavy chain as defined in SEQ ID NO: 19, 22 or 24 and a light chain as defined in SEQ ID NO: 28 or an anti-PD1 chimeric antibody comprising an heavy chain as defined is SEQ ID NO: 71 and a light chain as defined in SEQ ID NO: 72.

1-33. (canceled)

34. A bifunctional molecule comprising:
(a) an anti-human PD-1 antibody or an antigen-binding fragment thereof, which comprises:
(i) a heavy chain variable domain (VH) comprising a HCDR1, a HCDR2 and a HCDR3, and
(ii) a light chain variable domain (VL) comprising a LCDR1, a LCDR2 and a LCDR3, and
(b) a human interleukin 7 (IL-7) or a fragment or variant thereof, wherein the antibody or the fragment thereof is covalently linked to the human IL-7 or a fragment or variant thereof as a fusion protein.

35. The bifunctional molecule of claim 34, wherein the N-terminal end of the human IL-7 or the fragment thereof is connected to the C-terminal end of the heavy chain or of the light chain of the anti-human PD-1 antibody or the antigen-binding fragment thereof or both.

36. The bifunctional molecule of claim 34, wherein the antibody or the antigen-binding fragment thereof is a chimeric, a humanized or a human antibody.

37. The bifunctional molecule of claim 34, wherein the anti-human PD-1 antibody or antigen-binding fragment thereof, comprises:
(i) a heavy chain variable domain (VH) comprising HCDR1, HCDR2 and HCDR3, and
(ii) a light chain variable domain (VL) comprising LCDR1, LCDR2 and LCDR3, wherein:
the heavy chain CDR1 (HCDR1) comprises or consists of an amino acid sequence of SEQ ID NO: 1;
the heavy chain CDR2 (HCDR2) comprises or consists of an amino acid sequence of SEQ ID NO: 2;
the heavy chain CDR3 (HCDR3) comprises or consists of an amino acid sequence of SEQ ID NO: 3 wherein X1 is D or E and X2 is selected from the group consisting of T, H, A, Y, N, E and S;
the light chain CDR1 (LCDR1) comprises or consists of an amino acid sequence of SEQ ID NO: 12 wherein X is G or T;
the light chain CDR2 (LCDR2) comprises or consists of an amino acid sequence of SEQ ID NO: 15;
the light chain CDR3 (LCDR3) comprises or consists of an amino acid sequence of SEQ ID NO:16.

38. The bifunctional molecule of claim 34, wherein the anti-human PD-1 antibody or antigen-binding fragment thereof, comprises or consists of (a) a VH comprising or consisting of an amino acid sequence of SEQ ID NO: 17, wherein X1 is D or E and X2 is selected from the group consisting of T, H, A, Y, N, E and S; and (b) a VL comprising or consisting of an amino acid sequence of SEQ ID NO: 26, wherein X is G or T.

39. The bifunctional molecule of claim 34, wherein the anti-human PD-1 antibody or antigen-binding fragment thereof, comprises or consists of (i) a heavy chain variable region (VH) comprising or consisting of an amino acid sequence of SEQ ID NO: 24; and (ii) a light chain variable region (VL) comprising or consisting of an amino acid sequence of SEQ ID NO: 28.

40. The bifunctional molecule of claim 34, wherein, the anti-PD1 antibody is be selected from the group consisting of Pembrolizumab, Nivolumab, Pidilizumab, Cemiplimab, PDR001, and monoclonal antibodies 5C4, 17D8, 2D3, 4H1, 4A11, 7D3, and 5F4.

41. The bifunctional molecule of claim 34, wherein the IL-7 or the variant thereof comprises or consists of an amino acid sequence having at least 75% identity with a wild type human IL-7 (wth-IL-7).

42. The bifunctional molecule of claim 34, wherein the IL-7 comprises or consists of the amino acid sequence set forth in SEQ ID NO: 51.

43. The bifunctional molecule of claim 34, wherein the IL-7 is an IL-7 variant wherein the IL-7 variant presents at least 75% identity with a wild type human IL-7 (wth-IL-7) comprising or consisting of the amino acid sequence set forth in SEQ ID NO: 51, wherein the variant comprises at least one amino acid mutation which i) reduces affinity of the IL-7 variant for IL-7 receptor (IL-7R) in comparison to the affinity of wth-IL-7 for IL-7R, and ii) improves pharmacokinetics of the bifunctional molecule comprising the IL-7 variant in comparison with a bifunctional molecule comprising wth-IL-7.

44. The bifunctional molecule of claim 43, wherein the at least one mutation is an amino acid substitution or a group of amino acid substitutions selected from the group consisting of (i) C2S-C141S and C47S-C92S, C2S-C141S and C34S-C129S, or C47S-C92S and C34S-C129S, (ii) W142H, W142F or W142Y, (iii) D74E, D74Q or D74N, iv) Q11E, Y12F, M17L, Q22E and/or K81R; or any combination thereof.

45. The bifunctional molecule of claim 34, wherein the IL-7 variant comprises or consists of the amino acid sequence set forth in SEQ ID NO: 53-66.

46. The bifunctional molecule of claim 34, wherein the antibody or antigen-binding fragment thereof comprises a light chain constant domain derived from a human kappa light chain constant domain and a heavy chain constant domain derived from a human IgG1 heavy chain constant domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/I332E; P257I/Q311; K326W/E333S; S239D/I332E/G236A; N297A; L234A/L235A; N297A+M252Y/S254T/T256E; K322A; and K444A.

47. The bifunctional molecule of claim 34, wherein the antibody or antigen-binding fragment thereof comprises a light chain constant domain derived from a human kappa light chain constant domain and a heavy chain constant domain derived from a human IgG4 heavy chain constant domain, optionally with a substitution or a combination of substitutions selected from the group consisting of S228P, L234A/L235A, S228P+M252Y/S254T/T256E.17 and K444A.

48. The bifunctional molecule of claim 34, wherein the antibody or a fragment thereof is linked to IL-7 or a variant thereof by a linker sequence.

49. The bifunctional molecule of claim 45, wherein the antibody or antigen-binding fragment thereof comprises a light chain constant domain derived from a human kappa light chain constant domain and a heavy chain constant domain derived from a human IgG1 heavy chain constant domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/I332E; P257I/Q311; K326W/E333S; S239D/I332E/G236A; N297A; L234A/L235A; N297A+M252Y/S254T/T256E; K322A; and K444A; and the antibody or a fragment thereof is linked to IL-7 variant by a linker (GGGGS)₃.

50. An isolated nucleic acid molecule or a group of isolated nucleic acid molecules encoding the bifunctional molecule according to claim 34.

51. A vector comprising the nucleic acid or group of nucleic acid molecules according to claim 50.

52. A host cell comprising the nucleic acid or group of nucleic acid molecules of claim 50 or a vector comprising said nucleic acid or group of nucleic acids.

53. A method for producing the bifunctional molecule comprising a step of culturing a host cell according to claim 52 and optionally a step of isolating the bifunctional molecule.

54. A pharmaceutical composition comprising the bifunctional molecule according to of claim 34 and a pharmaceutically acceptable carrier.

55. A method of treating cancer comprising the administration of a pharmaceutical composition according to claim 54 to a subject in need of treatment.

56. The method of claim 55, wherein the cancer is a hematologic malignancy or a solid tumor with expression of PD-1 and/or PD-L1 selected from hematolymphoid neoplasms, angioimmunoblastic T cell lymphoma, myelodysplastic syndrome, and acute myeloid leukemia, a cancer induced by virus or associated with immunodeficiency, Kaposi sarcoma, cervical, anal, penile and vulvar squamous cell cancer, oropharyngeal cancers, B cell non-Hodgkin lymphomas (NHL), diffuse large B-cell lymphoma, Burkitt lymphoma, plasmablastic lymphoma, primary central nervous system lymphoma, HHV-8 primary effusion lymphoma, classic Hodgkin lymphoma, lymphoproliferative disorders, hepatocellular carcinoma, Merkel cell carcinoma, cancer associated with human immunodeficiency virus infection (HIV) infection, metastatic or non-metastatic cancer, Melanoma, malignant mesothelioma, Non-Small Cell Lung Cancer, Renal Cell Carcinoma, Hodgkin's Lymphoma, Head and Neck Cancer, Urothelial Carcinoma, Colorectal Cancer, Hepatocellular Carcinoma, Small Cell Lung Cancer, Metastatic Merkel Cell Carcinoma, Gastric or Gastroesophageal cancers and Cervical Cancer.

57. The method of claim 56, wherein said pharmaceutical composition is administered in combination with radiotherapy or an additional therapeutic agent selected from the group consisting of alkylating agents, angiogenesis inhibitors, antibodies, antimetabolites, antimitotics, antiproliferatives, antivirals, aurora kinase inhibitors, apoptosis promoters activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccines, epitopes or neoepitopes from tumor antigens, and combinations of one or more of these agents.

58. A method of treating infectious disease, chronic infectious disease, or chronic viral infections comprising the administration of a pharmaceutical composition according to claim 54 to a subject in need of treatment.

59. The method of claim 58, wherein the infectious disease is caused by a virus selected from the group consisting of HIV, hepatitis virus, herpes virus, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.


SEQUENCE LISTING (74 sequences)

SEQ ID NO: 1 - protein, 5 aa, artificial
  HC CDR1
HYAMN

SEQ ID NO: 2 - protein, 17 aa, artificial
  HC CDR2
WINTNTGEPTYAQGFTG

SEQ ID NO: 3 - protein, 8 aa, artificial
  HC CDR3
  MISC_FEATURE (7)..(7): X is X1 (D or E)
  MISC_FEATURE (8)..(8): X is X2 and is T, H, A, Y, N, E or S
EREPGMXX

SEQ ID NO: 4 - protein, 8 aa, artificial
  HC CDR3
EREPGMDS

SEQ ID NO: 5 - protein, 8 aa, artificial
  HC CDR3
EREPGMDT

SEQ ID NO: 6 - protein, 8 aa, artificial
  HC CDR3
EREPGMES

SEQ ID NO: 7 - protein, 8 aa, artificial
  HC CDR3
EREPGMDH

SEQ ID NO: 8 - protein, 8 aa, artificial
  HC CDR3
EREPGMDA

SEQ ID NO: 9 - protein, 8 aa, artificial
  HC CDR3
EREPGMDY

SEQ ID NO: 10 - protein, 8 aa, artificial
  HC CDR3
EREPGMDN

SEQ ID NO: 11 - protein, 8 aa, artificial
  HC CDR3
EREPGMDE

SEQ ID NO: 12 - protein, 16 aa, artificial
  LC CDR1
  MISC_FEATURE (11)..(11): X is G or T
RSSQSLVHANXNTYLE

SEQ ID NO: 13 - protein, 16 aa, artificial
  LC CDR1
RSSQSLVHANGNTYLE

SEQ ID NO: 14 - protein, 16 aa, artificial
  LC CDR1
RSSQSLVHANTNTYLE

SEQ ID NO: 15 - protein, 7 aa, artificial
  LC CDR2
KVSNRFS

SEQ ID NO: 16 - protein, 9 aa, artificial
  LC CDR3
FQGTHVPNT

SEQ ID NO: 17 - protein, 117 aa, artificial
  HC variable domain
  MISC_FEATURE (105)..(105): X is X1, D or E
  MISC_FEATURE (106)..(106): X is X2, and is T, H, A, Y, N, E or S
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMXXWGQGTLVTVSS

SEQ ID NO: 18 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSS

SEQ ID NO: 19 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSS

SEQ ID NO: 20 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSS

SEQ ID NO: 21 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSS

SEQ ID NO: 22 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSS

SEQ ID NO: 23 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSS

SEQ ID NO: 24 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSS

SEQ ID NO: 25 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSS

SEQ ID NO: 26 - protein, 112 aa, artificial
  LC variable domain
  MISC_FEATURE (34)..(34): X is G or T
DVVMTQSPLSLPVTLGQPASISCRSSQSLVHANXNTYLEWYQQRPGQSPRLLIYKVSNRFSGVPDRFSGSGSGTDFTLKISRVEAEDVGVYYCFQGTHVPNTFGQGTKLEIK

SEQ ID NO: 27 - protein, 112 aa, artificial
  LC variable domain
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIK

SEQ ID NO: 28 - protein, 112 aa, artificial
  LC variable domain
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIK

SEQ ID NO: 29 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 30 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 31 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 32 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 33 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 34 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 35 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 36 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 37 - protein, 219 aa, artificial
  LC
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 219 aa, artificial
  LC
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 327 aa, artificial
  HC constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 40 - protein, 107 aa, artificial [sequence omitted]

SEQ ID NO: 41 - protein, 30 aa, artificial
  HFR1
QIQLVQSGSELKKPGASVKVSCKASGYTFT

SEQ ID NO: 42 - protein, 14 aa, artificial
  HFR2
WVRQAPGQGLEWMG

SEQ ID NO: 43 - protein, 32 aa, artificial
  HFR3
RFVFSLDTSVSTAYLQISSLKAEDTAVYYCAR

SEQ ID NO: 44 - protein, 11 aa, artificial
  HFR4
WGQGTLVTVSS

SEQ ID NO: 45 - protein, 23 aa, artificial
  LFR1
DVVMTQSPLSLPVTLGQPASISC

SEQ ID NO: 46 - protein, 15 aa, artificial
  LFR2
WYQQRPGQSPRLLIY

SEQ ID NO: 47 - protein, 32 aa, artificial
  LFR3
GVPDRFSGSGSGTDFTLKISRVEAEDVGVYYC

SEQ ID NO: 48 - protein, 10 aa, artificial
  LFR4
FGQGTKLEIK

SEQ ID NO: 49 - protein, 19 aa, artificial
  signal peptide
MDWLWNLLFLMAAAQSIQA

SEQ ID NO: 50 - protein, 19 aa, artificial
  signal peptide
MKLPVRLLVLMFWIPASSS

SEQ ID NO: 51 - protein, 152 aa, artificial
  IL7
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 52 - protein, 330 aa, artificial
  Heavy chain constant domain (IgG1m-N298A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 152 aa, artificial
  IL7-SS1
DSDIEGKDGKQYESVLMVSIDQLLDSMKEIGSNSLNNEFNFFKRHICDANKEGMFLFRAARKLRQFLKMNSTGDFDLHLLKVSEGTTILLNCTGQVKGRKPAALGEAQPTKSLEENKSLKEQKKLNDLSFLKRLLQEIKTSWNKILMGTKEH

SEQ ID NO: 54 - protein, 152 aa, artificial
  IL7-SS2
DSDIEGKDGKQYESVLMVSIDQLLDSMKEIGSNCLNNEFNFFKRHISDANKEGMFLFRAARKLRQFLKMNSTGDFDLHLLKVSEGTTILLNSTGQVKGRKPAALGEAQPTKSLEENKSLKEQKKLNDLCFLKRLLQEIKTSWNKILMGTKEH

SEQ ID NO: 55 - protein, 152 aa, artificial
  IL7-SS3
DCDIEGKDGKQYESVLMVSIDQLLDSMKEIGSNSLNNEFNFFKRHISDANKEGMFLFRAARKLRQFLKMNSTGDFDLHLLKVSEGTTILLNSTGQVKGRKPAALGEAQPTKSLEENKSLKEQKKLNDLSFLKRLLQEIKTCWNKILMGTKEH

SEQ ID NO: 56 - protein, 152 aa, artificial
  IL7-W142H
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 57 - protein, 152 aa, artificial
  IL7-W142Y
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCYNKILMGTKEH

SEQ ID NO: 58 - protein, 152 aa, artificial
  IL7-W142F
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCFNKILMGTKEH

SEQ ID NO: 59 - protein, 152 aa, artificial
  IL7-Q11E
DCDIEGKDGKEYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 60 - protein, 152 aa, artificial
  IL7-Y12F
DCDIEGKDGKQFESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 61 - protein, 152 aa, artificial
  IL7-M17L
DCDIEGKDGKQYESVLLVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 62 - protein, 152 aa, artificial
  IL7-Q22E
DCDIEGKDGKQYESVLMVSIDELLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 63 - protein, 152 aa, artificial
  IL7-D74E
DCDIEGKDGKQYESVLMVSIDQLLDSMKEIGSNCLNNEFNFFKRHICDANKEGMFLFRAARKLRQFLKMNSTGEFDLHLLKVSEGTTILLNCTGQVKGRKPAALGEAQPTKSLEENKSLKEQKKLNDLCFLKRLLQEIKTCWNKILMGTKEH

SEQ ID NO: 64 - protein, 152 aa, Artificial Sequence
  IL7-D74Q
DCDIEGKDGKQYESVLMVSIDQLLDSMKEIGSNCLNNEFNFFKRHICDANKEGMFLFRAARKLRQFLKMNSTGQFDLHLLKVSEGTTILLNCTGQVKGRKPAALGEAQPTKSLEENKSLKEQKKLNDLCFLKRLLQEIKTCWNKILMGTKEH

SEQ ID NO: 65 - protein, 152 aa, artificial
  IL7-D74N
DCDIEGKDGKQYESVLMVSIDQLLDSMKEIGSNCLNNEFNFFKRHICDANKEGMFLFRAARKLRQFLKMNSTGNFDLHLLKVSEGTTILLNCTGQVKGRKPAALGEAQPTKSLEENKSLKEQKKLNDLCFLKRLLQEIKTCWNKILMGTKEH

SEQ ID NO: 66 - protein, 152 aa, artificial
  IL7-K81R
DCDIEGKDGKQYESVLMVSIDQLLDSMKEIGSNCLNNEFNFFKRHICDANKEGMFLFRAARKLRQFLKMNSTGDFDLHLLRVSEGTTILLNCTGQVKGRKPAALGEAQPTKSLEENKSLKEQKKLNDLCFLKRLLQEIKTCWNKILMGTKEH

SEQ ID NO: 67 - protein, 1 aa, artificial
  linker C
C

SEQ ID NO: 68 - protein, 5 aa, artificial
  linker G4S
GGGGS

SEQ ID NO: 69 - protein, 10 aa, artificial
  linker (G4S)2
GGGGSGGGGS

SEQ ID NO: 70 - protein, 15 aa, artificial
  linker (G4S)3
GGGGSGGGGSGGGGS

SEQ ID NO: 71 - protein, 444 aa, artificial
  HC anti-PD1 chimeric
QIQLVQSGPELKKPGETVKISCKASGYTFTHYGMNWVKQAPGKGLKWMGWINTNTGEPTYAEEFKGRFAFSLETSASAAFLQINNLKNEDTATYFCAKEREPGMDSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 72 - protein, 219 aa, artificial
  LC anti-PD1 chimeric
DVLLTQTPLSLPVSLGDQASISCRSSQNIVHANGNTYLEWYLQKPGQSPKLLIYKVSNRFSGVPDRFSGSGSGTDFTLKITRVEAEDLGVYYCFQGSHVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 2670 bp, artificial
  VH-VK
cagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatgacagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 74 - DNA, 660 bp, artificial
  VL-VD
gacgtggtcatgacacagagcccactgtctctgcctgtgaccctgggacagccagcctctatctcctgcagatccagccagtctctggtgcacgctaacaccaatacatacctggagtggtatcagcagaggccaggacagtccccaaggctgctgatctacaaggtgtccaacagattcagcggagtgccagaccgctttagcggatctggatccggaaccgacttcaccctgaagatctccagggtggaggctgaggatgtgggcgtgtactattgtttccagggcacccatgtgcctaatacatttggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag